(19) United States
(12) Patent Application Publication
(10) Pub. No.: US 2026/0248887 A1
(43) Pub. Date: Aug. 27, 2026

(54) Compositions and Methods for Treatment of Muscle Disease with Chronic Inflammation

(71) Applicant: New York Society for the Relief of the Ruptured and Crippled, maintaining the Hospital for Special, New York, NY (US)

(72) Inventors: Lan Zhou, New York, NY (US); Yinhang Wang, New York, NY (US); Xingyu Wang, New York, NY (US)

(21) Appl. No.: 19/532,382

(22) Filed: Feb. 6, 2026

Related U.S. Application Data
(60) Provisional application No. 63/754,822, filed on Feb. 6, 2025.

(51) Int. Cl. A61K 38/19; A61K 9/00; A61P 21/00

(52) U.S. Cl. CPC A61K 38/195; A61K 9/0019; A61P 21/00

(57) ABSTRACT

Provided herein are compositions, uses thereof and methods for treating muscular dystrophy and/or chronic inflammatory myopathy. The composition comprises at least one compound inhibiting CCR2 signaling and at least one compound inhibiting CSF-1R signaling.

### STATEMENT REGARDING FEDERALLY SPONSORED RESEARCH OR DEVELOPMENT

This invention was made with government support under grant number 1R01AR074428 awarded by the National Institute of Health. The government has certain rights in the invention.

### BACKGROUND OF THE INVENTION

Duchenne muscular dystrophy (DMD) is a lethal genetic disease with no effective cure currently. DMD is the most common genetic muscle disease caused by mutations in the dystrophin gene on the X chromosome, which leads to muscle membrane instability, myofiber necrosis, endomysial inflammation, fibrofatty tissue deposition, muscle weakness, and premature death (D. Duan, N. Goemans, S. Takeda, E. Mercuri, A. Aartsma-Rus, Duchenne muscular dystrophy. Nat Rev Dis Primers 7, 13 (2021); A. E. H. Emery, Duchenne Muscular Dystrophy (Oxford University Press, Oxford, 1993); E. P. Hoffman, R. H. Brown, Jr., L. M. Kunkel, Dystrophin: the protein product of the Duchenne muscular dystrophy locus. Cell 51, 919-928 (1987).). The disease has no cure at this point. Scientists in the DMD research field have been actively exploring therapeutic approaches to overcome the genetic defects by gene therapies, replace defective cells by cell therapies, and reduce muscle damage, inflammation, and fibrosis by pharmacotherapies. Muscle biopsy from patients with DMD features chronic inflammation and fibrosis, which contributes to muscle dysfunction and clinical weakness (I. Desguerre et al., Endomysial fibrosis in Duchenne muscular dystrophy: a marker of poor outcome associated with macrophage alternative activation. Journal of neuropathology and experimental neurology 68, 762-773 (2009)).

DMD has no cure at this point. Current clinical studies exploring therapeutic approaches include 1) overcoming the genetic defects by gene therapies, 2) replacing defective cells by cell therapies, and 3) reducing muscle damage, inflammation, and fibrosis by pharmacotherapies. The effort of gene therapy is challenged by large size of dystrophin gene to be manipulated, the delivery efficiency of the ectopic gene, and immune response against widely used adenoviral vectors. Cell therapy also faces several obstacles, including low efficiency of cell engraftment and the immune response against transplanted cells. Anti-inflammatory pharmacotherapies can not only ameliorate muscle inflammation and fibrosis, and improves muscle function, but also improves local tissue environment to enhance gene and cell engraftment efficiency. Pharmacotherapies represent a necessary addition to the gene and cell therapies for DMD.

Currently, glucocorticoids and histone deacetylase (HDAC) inhibitor are the main pharmacological drugs to reduce muscle inflammation in DMD. However, prolonged use of glucocorticoids can cause weight gain, weakened bones, and an increased risk of fractures.

There is not standard treatment or cure for muscular dystrophy disorders. A continuing need in the art exists for compositions and methods for effective treatment for muscular dystrophy disorders.

### SUMMARY OF THE INVENTION

In one aspect, provided herein is a method for treating chronic muscle inflammation, the method comprising systemic administration to subject in a need thereof a therapeutically effective amount of at least one compound that disrupts a monocyte recruitment and/or macrophage expansion in response to muscle injury. In certain embodiments, the systemic administration comprises oral administration, subcutaneous injection, and/or intravenous injection. In certain embodiments, the method comprises disrupting monocyte recruitment and macrophage expansion in response to muscle injury. In certain embodiments, the chronic muscle inflammation is associated with muscular dystrophy, optionally Duchenne Muscular Dystrophy (DMD). In certain embodiments, the at least one compound is a small molecule inhibitor, a peptide inhibitor, a protein inhibitor, an antibody, optionally a neutralizing antibody, a nucleic acid molecule inhibitor, optionally a small interfering RNA (siRNA). In certain embodiments, the method comprises disrupting monocyte recruitment, optionally wherein the method comprises blocking recruitment of blood Ly6Chi inflammatory monocyte by injured muscle. In certain embodiments, the method comprises decreasing Ly6Chi macrophage infiltration. In certain embodiments, the at least one compound inhibits C-C motif chemokine receptor 2 (CCR2) signaling. In certain embodiments, the at least one compound is a CCR2 inhibitor. In certain embodiments, the at least one compound is a C-C motif chemokine ligand 2 (CCL2; MCP-1), C-C motif chemokine ligand 8 (CCL8; MCP-2), C-C motif chemokine ligand 7 (CCL7; MCP-3), and/or C-C motif chemokine ligand 13 (CCL13; MCP-4) inhibitor. In certain embodiments, the at least one compound disrupts CCR2-CCL2 binding. In certain embodiments, wherein the method comprises disrupting macrophage expansion. In certain embodiments, wherein the method comprises decreasing Ly6Clo macrophage expansion. In certain embodiments, wherein the at least one compound inhibits a colony stimulating factor-1 receptor (CSF-1R) signaling. In certain embodiments, the at least one compound is a CSF-1R inhibitor. In certain embodiments, the at least one compound is a colony stimulating factor-1 (CSF-1) inhibitor. In certain embodiments, the at least one compound disrupts CSF-1R-CSF1 binding. In certain embodiments, the at least one compound is a CCR2 inhibitor that is PF-04136309, TAK-652, RS504393, MK0812, PF-04634817, or INCB3344. In certain embodiments, the at least one compound is a CSF-1R inhibitor that is PLX3397, PLX5622, BLZ945, or PLX73086. In certain embodiments, the method comprises administering to subject in a need thereof a therapeutically effective amount of at least one compound that inhibits CCR2 signaling and at least one compound that inhibits CSF-1R signaling.

In another aspect provided herein is a regimen for treating chronic muscle inflammation, the regimen comprising administering to subject in a need thereof a therapeutically effective amount of at least one compound that disrupts a monocyte recruitment in response to muscle injury and at least one compound that disrupts macrophage expansion in response to muscle injury. In certain embodiments, the regimen comprises administering of the at least one compound that disrupts a monocyte recruitment and at least one compound that disrupts macrophage expansion simultaneously. In certain embodiments, the regimen comprises first administering of the at least one compound that disrupts a monocyte recruitment, followed by administering at least one compound that disrupts macrophage expansion. In certain embodiments, the at least one compound inhibits C-C motif chemokine receptor 2 (CCR2) signaling. In certain embodiments, the at least one compound inhibits a colony stimulating factor-1 receptor (CSF-1R) signaling.

These and other aspects of the invention are apparent from the following detailed description of the invention.

### BRIEF DESCRIPTION OF THE FIGURES

FIGS. 1A-1E show that Nur77 deficiency diminishes blood Ly6C^{lo} monocytes but does not affect the density of Ly6C^{hi} or Ly6C^{lo} macrophages in mdx^{5cv} or mdx^{5cv}/Ccr2^{-/-} quadriceps and diaphragm. FIG. 1A shows represented dot plots of FACS analysis showing Ly6C^{hi} and Ly6C^{lo} blood monocytes (MOS) in mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age. FIG. 1B shows bar graph showing densities of blood MOs quantified from FACS analysis. N=5 mice/group. ****p<0.0001. FIG. 1C to E show bar graph showing densities of intramuscular Ly6C^{hi} (C), Ly6C^{lo} (FIG. 1D), and total (FIG. 1E) macrophages (MPs) quantified by flow cytometry in the diaphragm and quadriceps of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at ages 6 weeks, 14 weeks, and 24 weeks. N=10 mice/group. **p<0.01; ***p<0.001; ****p<0.0001; ns: no significance.

FIGS. 2A-2C show embryo-derived Ly6C^{lo} resident macrophages expand in mdx^{5cv}/Ccr2^{-/-} quadriceps and diaphragm. FIG. 2A shows scheme of Flt3^{Cre}/Rosa26^{LSL-YFP} lineage tracing. FIG. 2B shows dot plots showing gating strategy for FACS identification of YFP⁺ (CD45⁺/CD64⁺/Ly6C^{lo}/YFP⁺) and YFP⁻ (CD45⁺/CD64⁺/Ly6C^{lo}/YFP⁻) Ly6C^{lo} macrophages with skeletal muscle single-cell suspensions. FIG. 2C shows bar graph showing densities of intramuscular YFP⁺ and YFP⁻ Ly6C^{lo} macrophages in the quadriceps (Qua) and diaphragm (Dia) of Flt3^{Cre}/Rosa26^{LSL-YFP} (FR), Flt3^{Cre}/Rosa26^{LSL-YFP}/mdx^{5cv} (FR/mdx^{5cv}), Flt3^{Cre}/Rosa26^{LSL-YFP}/mdx^{5cv}/Ccr2^{-/-} (FR/mdx^{5cv}/Ccr2^{-/-}), and Flt3^{Cre}/Rosa26^{LSL-YFP}/mdx^{5cv}/Ccr2^{-/-}/Nur77^{-/-} (FR/mdx^{5cv}/Ccr2^{-/-}/Nur77^{-/-}) mice at 14 weeks of age. N=10 mice/group. ***p<0.001; ****p<0.0001; ns: no significance.

FIGS. 3A-3C show macrophages in dystrophic mdx^{5cv} skeletal muscles contain multiple functional sub-clusters, among which the resident macrophage cluster expand in the absence of CCR2. Single-cell suspensions prepared from the quadriceps and diaphragm of mdx^{5cv} and mdx^{5cv}/Ccr2^{-/-} mice at 14 weeks of age were subjected to single-cell based RNA sequencing (scRNAseq) analysis. FIG. 3A shows Uniform Manifold Approximation and Projection (UMAP) dimension reduction analysis identifying sub-clusters of monocytes/macrophages in the quadriceps and diaphragm of mdx^{5cv} and mdx^{5cv}/Ccr2^{-/-} mice. FIG. 3B shows violin plots showing featured gene expression by different monocyte/macrophage sub-clusters in the quadriceps and diaphragm of mdx^{5cv} and mdx^{5cv}/Ccr2^{-/-} mice. FIG. 3C shows bar graphs showing the fraction of each sub-cluster to total monocytes/macrophages.

FIGS. 4A-4D show intramuscular macrophages express a similar level of the genes regulating inflammation and fibrosis between mdx^{5cv} and mdx^{5cv}/Ccr2^{-/-} quadriceps or diaphragm. Violin plots of scRNAseq data showing the expression of pro-inflammatory genes (FIG. 4A), anti-inflammatory genes (FIG. 4B), pro-fibrotic genes (FIG. 4C), and fibrosis regulatory genes (FIG. 4D) by macrophages of different muscle samples as indicated.

FIGS. 5A-5B show the expansion of intramuscular Ly6C^{lo} macrophages is resulted from increased proliferation in dystrophic skeletal muscles in the absence of CCR2. Single-cell suspensions prepared from the quadriceps and diaphragm of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age were subjected to FACS analysis of proliferation by EdU incorporation assay (FIG. 5A) and apoptosis by Vybrant Dye staining (FIG. 5B). FIG. 5A shows bar graphs comparing the percentages of EdU+macrophages in the quadriceps and diaphragm muscles among mice with different genotypes. FIG. 5B shows bar graphs showing comparisons of the percentages of apoptotic macrophages in the quadriceps and diaphragm muscle among mice with different genotypes. N=10 mice/group. ***p<0.001; ns: no significance.

FIGS. 6A-6I show a high intramuscular density of FAPs contributes to the increased CSF-1 production in mdx^{5cv}/Ccr2^{-/-} quadriceps and diaphragm. FIG. 6A and FIG. 6B show whole-muscle production of CSF-1 in the quadriceps and diaphragm between mdx^{5cv} and mdx^{5cv}/Ccr2^{-/-} mice at 14 weeks of age was determined by qRT-PCR for mRNA (FIG. 6A) and by ELISA for protein (FIG. 6B). N=10 mice/group. **p<0.01; ***p<0.001; ****p<0.0001. FIG. 6C shows violin plot of scRNAseq data showing the expression of Csflr by different monocyte macrophage sub-clusters. FIG. 6D shows feature plots of scRNAseq data showing that FAPs and neutrophils (arrows) are the major cellular sources of Csfl mRNA expression in the quadriceps (Qua) and diaphragm (Dia) of mdx^{5cv} and mdx^{5cv}/Ccr2^{-/-} mice. FIG. 6E and FIG. 6F show bar graphs showing comparisons of the cell densities, quantified by FACS analysis, of neutrophils (Neu) (FIG. 6E) and FAPs (FIG. 6F) in the quadriceps and diaphragm between mdx^{5cv} and mdx^{5cv}/Ccr2^{-/-} mice. N=10 mice/group. **p<0.01; ***p<0.001; ****p<0.0001; ns, no significance. FIG. 6G shows violin plot of scRNAseq data showing FAP expression of Csfl mRNA in the quadriceps and diaphragm of mdx^{5cv} and mdx^{5cv}/Ccr2^{-/-} mice. FIG. 6H shows qRT-PCR analysis of Csfl mRNA expression by FAPs sorted from the quadriceps and diaphragm of mdx^{5cv} and mdx^{5cv}/Ccr2^{-/-} mice at 14 weeks of age. FAPs were sorted from 5 mice/group and combined for RNA preparation. FIG. 6I show histogram of FACS analysis showing the expression of CSF-1 by FAPs in the quadriceps and diaphragm of mdx^{5cv} and mdx^{5cv}/Ccr2^{-/-} mice at 14 weeks of age. Black line: IgG isotype control (mdx^{5cv}). Black line with grey filled: IgG isotype control (mdx^{5cv}/Ccr2^{-/-}). Red line: CSF-1 staining (mdx^{5cv}). Blue line: CSF-1 staining (mdx^{5cv}/Ccr2^{-/-}). Data presented represents 5 mice/group.

FIGS. 7A-7F show FAPs in mdx^{5cv}/Ccr2^{-/-} quadriceps and diaphragm stimulate macrophage proliferation via CSF-1/CSF-1R signaling. FIG. 7A and FIG. 7B show circos plots of scRNAseq data showing well-knowing ligand-receptor pairs between FAPs (sender) and macrophages (receiver) in mdx^{5cv}/Ccr2^{-/-} quadriceps (FIG. 7A) and diaphragm (FIG. 7B) at 14 weeks of age. FAPs communicate with macrophages through CSF-1/CSF-1R pairing. FIG. 7C and FIG. 7D show FACS analysis of EdU incorporation showing proliferation of WT bone marrow-derived macrophages (BMDMs) stimulated by the conditioned medium of mdx^{5cv}/Ccr2^{-/-} FAPs in the absence or presence of CSF-1R inhibitor PLX3397. BMDMs cultured in culture medium without conditioned medium of mdx^{5cv}/Ccr2^{-/-} FAPs and PLX3397 were used as control. (C) Dot plots represent results with FAP-conditioned medium prepared from 5 individual mice (N=5). FIG. 7D shows bar graph showing quantified data of (FIG. 7C). ****p<0.0001. FIG. 7E & FIG. 7F shows bar graph showing densities of intramuscular Ly6C^{lo} macrophages quantified by flow cytometry in the quadriceps (FIG. 7E) and diaphragm (FIG. 7F) of mdx^{5cv}/Ccr2^{-/-} mice receiving PLX5622 or vehicle control. N≥6 mice/group. ****p<0.0001.

FIG. 8A shows dot plots of single-cell RNA sequencing (scRNAseq) data showing the expression of marker genes by different cell types in each muscle sample. FIG. 8B shows uniform Mani-fold Approximation and Projection (UMAP) dimension reduction analysis of scRNAseq data showing different cell types in mdx^{5cv} and mdx^{5cv}/Ccr2^{-/-} quadriceps and diaphragm muscles. FIG. 8C shows heatmap depicting top 10 differentially expressed genes (DEGs) of individual cell types in each muscle sample. The names of the top 10 DEGs of each cell type are listed in FIG. 18. ECs: Endothelial cell, DCs: Dendritic Cell, MO/MPs: Monocyte/Macrophage, FAPs:

Fibro/adipogenic progenitors, T:T cells, NK: Natural killer cells, NKT: invariant Natural Killer T Cells, MC: Mesothelial cells, SCs: Satellite cells.

FIG. 9 shows heatmap depicting top 10 differentially expressed genes in individual monocyte and macrophage clusters in mdx^{5cv} and mdx5cv/Ccr2−/− quadriceps and diaphragm muscles.

FIGS. 10A and 10B show UMAP analysis of scRNAseq data showing macrophage subclusters in wild-type, mdx^{5cv} and mdx^{5cv}/Ccr2^{-/-} quadriceps (Qua) and diaphragm (Dia) muscles. FIG. 10C shows bar graphs showing the percentage of each macrophage subcluster quantified from scRNAseq data. FIG. 10D shows heatmap depicting top 10 differentially expressed genes in individual monocyte/macrophage sub-clusters wild-type, mdx^{5cv} and mdx^{5cv}/Ccr2^{-/-} quadriceps and diaphragm muscles.

FIG. 11 shows violin plots showing the expression of Ccr2^{-/-}, Folr2, Timd4, and Lyve1 by different macrophage clusters in the quadriceps and diaphragm of mdx^{5cv} and mdx^{5cv}/Ccr2^{-/-} mice at 14 weeks of age.

FIG. 12 shows volcano plots comparing the transcriptome of macrophages between mdx^{5cv} and mdx^{5cv}/Ccr2^{-/-} quadriceps (FIG. 12A) and diaphragm (FIG. 12B). Differentially expressed genes (DEGs, p<0.05, Log 2FC≥0.5) are indicated.

FIG. 13A and FIG. 13B show violin plot of scRNAseq data showing the expression of Igfl (FIG. 13A) and Timp2 (FIG. 13B) gene by different macrophage subclusters. FIG. 13C to F show quantitative reverse transcription-PCR (qRT-PCR) analysis of the mRNA expression of pro-inflammatory (FIG. 13C), anti-inflammatory (FIG. 13D), pro-fibrotic (FIG. 13E) and fibrosis regulatory (FIG. 13F) genes by YFP⁺ and YFP-Ly6C^{lo} macrophages in Flt3^{Cre}/Rosa26^{LSL-YFP}/mdx^{5cv}/Ccr2^{-/-} quadriceps (Qua) and diaphragm (Dia). YFP+and YFP Ly6C^{lo} macrophages each were sorted and pooled from 10Flt3^{Cre}/Rosa26^{LSL-YFP}/mdx^{5cv}/Ccr2^{-/-} mice. PCR analysis was performed at triplicates.

FIG. 14A and FIG. 14B show single-cell suspensions prepared from the quadriceps and diaphragm of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age were subjected to FACS analysis of proliferation by EdU incorporation assay (FIG. 14A) and apoptosis by Vybrant Dye staining (FIG. 14B). FIG. 14A shows represented dot plots showing EdU+ proliferating macrophages. FIG. 14B shows represented dot plots showing apoptotic macrophages. FIG. 14C shows represented dot plots showing apoptosis of BMDMs induced by actinomycin D treatment (0.5 mM, 12 hours) or by CSF-1 starvation (24 hours), and apoptosis of mouse splenic CD3⁺ T cells. FIG. 14D shows qRT-PCR analysis of the mRNA expression of Il4, Il34 and Spp1 by mdx^{5cv} and mdx^{5cv}/Ccr2^{-/-} quadriceps and diaphragm. n=10 per group. ns: no significance.

FIG. 15A shows heatmap depicting top 10 differentially expressed genes in individual FAP subclusters in mdx^{5cv} and mdx^{5cv}/Ccr2^{-/-} quadriceps and diaphragm muscles. FIGS. 15B and 15C UMAP analysis of scRNAseq data showing FAP subclusters in mdx^{5cv} and mdx^{5cv}/Ccr2^{-/-} quadriceps (Qua) and diaphragm (Dia) muscles. FIG. 15D shows bar graphs showing the percentage of each FAP sub-cluster quantified from scRNAseq data. FIG. 15E shows violin plot showing the expression of Csfl gene by each sub-clusters in mdx^{5cv} and mdx^{5cv}/Ccr2^{-/-} quadriceps (Qua) and diaphragm (Dia) muscles.

FIGS. 16A to 16B show Circos plots showing well-knowing ligand-receptor pairs of FAPs (sender) and macrophages (receiver) in mdx^{5cv} quadriceps (FIG. 16A) and diaphragm (FIG. 16B) muscles at 14 weeks of age. The FAPs communicate with macrophages through CSF-1/CSF-1R signal pathway. FIG. 16C shows FACS analysis of CSFE dilution showing proliferation of WT bone marrow derived macrophages (BMDMs) stimulated by the conditioned medium of mdx^{5cv}/Ccr2^{-/-} FAPs in the absence or presence of CSF-1R inhibitor PLX3397. Data represents results with FAP-conditioned medium prepared from 5 individual mice (N=5). FIG. 16D Immunostaining of CD68 using cryo-sections of quadriceps (Qua) and diaphragm (Dia) from mdx^{5cv}/Ccr2^{-/-} mice treated with PLX5622 or Vehicle control to determine the density of intramuscular macrophages. Sample pictures (Left) showing positive staining (as exampled by red arrows). Quantified data is shown as dot plots (Right). N≥8. ****p<0.0001.

FIG. 17 shows a table describing scRNAseq Quality Control.

FIG. 18 shows a table describing top 10 differentially expressed genes by different intramuscular cell types in quadriceps (Qua) and diaphragm (Dia) muscles of mdx^{5cv} and mdx^{5cv}/Ccr2^{-/-} mice.

FIG. 19 shows a table describing number and percentage of each cell type identified by scRNAseq in mdx^{5cv} and mdx^{5cv}/Ccr2^{-/-} quadriceps and diaphragm muscles.

FIG. 20 shows a table describing top 50 differentially expressed genes of monocytes and macrophage sub-clusters in mdx^{5cv} and mdx^{5cv}/Ccr2^{-/-} quadriceps and diaphragm muscles.

FIG. 21 shows a table describing number and percentage of individual monocytes and macrophage sub-clusters identified by scRNAseq.

FIG. 22 shows a table describing top 50 differentially expressed genes of monocytes and macrophage sub-clusters in wildtype, mdx^{5cv} and mdx^{5cv}/Ccr2^{-/-} quadriceps and diaphragm muscles.

FIG. 23 shows a table describing number and percentage of monocytes and individual sub-cluster of macrophages identified by scRNAseq in wild-type (WT), mdx^{5cv} and mdx^{5cv}/Ccr2^{-/-} quadriceps and diaphragm muscles.

FIG. 24 shows a table describing top 50 differentially expressed genes of FAP sub-clusters in mdx^{5cv} and mdx^{5cv}/Ccr2^{-/-} quadriceps and diaphragm muscles.

FIGS. 25A and 25B show quantified results of flow cytometry analysis of intramuscular macrophages from the quadriceps and diaphragm of (FIG. 25A) mdx^{5cv}/Ccr2^{-/-} mice receiving CCR2 inhibitor PF-4136309 and (FIG. 25B) mdx^{5cv} mice receiving both CCR2 inhibitor PF-4136309 and CSF-1R inhibitor PLX5622.

### DETAILED DESCRIPTION OF THE INVENTION

Provided herein are methods and regimens for treating chronic muscle inflammation, wherein the method and/or regimen comprises systemic administration to subject in a need thereof a therapeutically effective amount of at least one compound that disrupts a monocyte recruitment and/or macrophage expansion in response to muscle injury. Also provided herein are compositions for use in methods and regimens as described herein.

The term chronic muscle inflammation refers to condition that causes muscle weakness and inflammation the causes of which include, but not limited to injury, infection and autoimmune disease (see also, my. clevelandclinic. org/health/diseases/24170-myositis, which is incorporated herein by reference). In certain embodiments, the chronic muscle inflammation is associated with muscular dystrophy. In certain embodiments, the chronic muscle inflammation is associated with Duchenne muscular dystrophy (DMD). DMD is a lethal genetic disease with no effective cure currently. DMD is the most common genetic muscle disease caused by mutations in the dystrophin gene on the X chromosome, which leads to muscle membrane instability, myofiber necrosis, endomysial inflammation, fibrofatty tissue deposition, muscle weakness, and premature death (D. Duan, N. Goemans, S. Takeda, E. Mercuri, A. Aartsma-Rus, Duchenne muscular dystrophy. Nat Rev Dis Primers 7, 13 (2021); A. E. H. Emery, Duchenne Muscular Dystrophy (Oxford University Press, Oxford, 1993); E. P. Hoffman, R. H. Brown, Jr., L. M. Kunkel, Dystrophin: the protein product of the Duchenne muscular dystrophy locus. Cell 51, 919-928 (1987)). Muscle biopsy from patients with DMD features chronic inflammation and fibrosis, which contributes to muscle dysfunction and clinical weakness (I. Desguerre et al., Endomysial fibrosis in Duchenne muscular dystrophy: a marker of poor outcome associated with macrophage alternative activation. Journal of neuropathology and experimental neurology 68, 762-773 (2009).).

Ameliorating muscle inflammation and fibrosis can improve muscular dystrophy phenotype and enhance gene and cell engraftment efficiency for DMD. Preclinical studies by our lab and others demonstrate that ameliorating muscle inflammation and fibrosis improves muscle function (P. Huang, X. S. Zhao, M. Fields, R. M. Ransohoff, L. Zhou, Imatinib attenuates skeletal muscle dystrophy in mdx mice. The FASEB journal: official publication of the Federation of American Societies for Experimental Biology 23, 2539-2548 (2009); K. D. Huebner, D. S. Jassal, O. Halevy, M. Pines, J. E. Anderson, Functional resolution of fibrosis in mdx mouse dystrophic heart and skeletal muscle by halofuginone. American journal of physiology. Heart and circulatory physiology 294, H1550-1561 (2008); C. F. Spurney et al., Losartan decreases cardiac muscle fibrosis and improves cardiac function in dystrophin-deficient mdx mice. Journal of cardiovascular pharmacology and therapeutics 16, 87-95 (2011); A. P. Taniguti, A. Pertille, C. Y. Matsumura, H. Santo Neto, M. J. Marques, Prevention of muscle fibrosis and myonecrosis in mdx mice by suramin, a TGF-betal blocker. Muscle Nerve 43, 82-87 (2011); T. Turgeman et al., Prevention of muscle fibrosis and improvement in muscle performance in the mdx mouse by halofuginone. Neuromuscul Disord 18, 857-868 (2008); J. Capote et al., Osteopontin ablation ameliorates muscular dystrophy by shifting macrophages to a pro-regenerative phenotype. J Cell Biol 213, 275-288 (2016); S. A. Villalta et al., Interleukin-10 reduces the pathology of mdx muscular dystrophy by deactivating M1 macrophages and modulating macrophage phenotype. Hum Mol Genet 20, 790-805 (2011)). It also improves local tissue environment to enhance gene and cell engraftment efficiency (L. Cordier et al., Rescue of skeletal muscles of gamma-sarcoglycan-deficient mice with adeno-associated virus-mediated gene transfer. Molecular therapy: the journal of the American Society of Gene Therapy 1, 119-129 (2000); C. Gargioli, M. Coletta, F. De Grandis, S. M. Cannata, G. Cossu, PIGF-MMP-9-expressing cells restore microcirculation and efficacy of cell therapy in aged dystrophic muscle. Nature medicine 14, 973-978 (2008)). Therefore, anti-inflammatory and anti-fibrotic therapies may represent a necessary addition to the gene and cell therapies for DMD. Muscle inflammation and fibrosis appear linked in DMD. Chronic inflammation can cause collateral muscle damage and contribute to muscle fibrosis, as inflammatory cells can produce pro-fibrotic factors, such as TGF-β1, PDGF, and osteopontin to activate tissue fibrogenic cells to produce and deposit excessive extracellular matrix (ECM) proteins, including collagens and fibronectin, leading to fibrosis (T. A. Wynn, K. M. Vannella, Macrophages in Tissue Repair, Regeneration, and Fibrosis. Immunity 44, 450-462 (2016); L. Zhou et al., Temporal and spatial mRNA expression patterns of TGF-beta1, 2, 3 and TbetaRI, II, III in skeletal muscles of mdx mice. Neuromuscul Disord 16, 32-38 (2006). ; L. E. Olson, P. Soriano, Increased PDGFRalpha activation disrupts connective tissue development and drives systemic fibrosis. Dev Cell 16, 303-313 (2009).).

Muscle inflammation in DMD patients and mouse models is predominated by macrophage infiltration. A large portion of inflammatory cells in DMD muscle biopsy are macrophages. Mdx or Mdx5cv mice, the most commonly used DMD animal models, show persistent inflammation and progressive fibrosis in diaphragm, resembling dystrophic muscles in human DMD patients (G. Goldspink, K. Fernandes, P. E. Williams, D. J. Wells, Age-related changes in collagen gene expression in the muscles of mdx dystrophic and normal mice. Neuromuscul Disord 4, 183-191 (1994); J. V. Hartel, J. A. Granchelli, M. S. Hudecki, C. M. Pollina, L. E. Gosselin, Impact of prednisone on TGF-betal and collagen in diaphragm muscle from mdx mice. Muscle Nerve 24, 428-432 (2001); H. H. Stedman et al., The mdx mouse diaphragm reproduces the degenerative changes of Duchenne muscular dystrophy. Nature 352, 536-539 (1991)).

We demonstrate reduction of inflammation by specifically suppressing the pathogenic accumulation of macrophages through selective inhibition of CCR2 and CSF-1R. The method is selective and effective, showing good tolerability in our animal study.

In one aspect, provided herein is a method for treating chronic muscle inflammation, wherein the method comprises systemic administration to subject in a need thereof a therapeutically effective amount of at least one compound that disrupts a monocyte recruitment and/or macrophage expansion in response to muscle injury, wherein the method comprises disrupting monocyte recruitment and macrophage expansion in response to a muscle injury.

In one aspect, provided herein is a method for treating chronic muscle inflammation, wherein the method comprises systemic administration to subject in a need thereof a therapeutically effective amount of at least one compound that disrupts a monocyte recruitment and/or macrophage expansion in response to muscle injury, wherein the method comprises disrupting monocyte recruitment. In certain embodiments, the method comprises blocking recruitment of blood Ly6C^{hi} inflammatory monocyte by injured muscle. In certain embodiments, the method comprises decreasing Ly6C^{hi} macrophage infiltration.

In certain embodiments, the at least one compound inhibits C-C motif chemokine receptor 2 (CCR2) signaling. In certain embodiments, the at least one compound is a CCR2 inhibitor. In certain embodiments, the at least one compound is a C-C motif chemokine ligand 2 (CCL2; MCP-1) inhibitor. In certain embodiments, the at least one compound is an inhibitor that disrupts CCR2-CCL2 binding. In certain embodiments, the at least one compound is a C-C motif chemokine ligand 8 (CCL8; MCP-2) inhibitor. In certain embodiments, the at least one compound is a C-C motif chemokine ligand 7 (CCL7; MCP-3) inhibitor. In certain embodiments, the at least one compound is a C-C motif chemokine ligand 13 (CCL13; MCP-4) inhibitor.

In one aspect, provided herein is a method for treating chronic muscle inflammation, wherein the method comprises systemic administration to subject in a need thereof a therapeutically effective amount of at least one compound that disrupts a monocyte recruitment and/or macrophage expansion in response to muscle injury, wherein the method the method comprises disrupting macrophage expansion.

In one aspect, provided herein is a method for treating chronic muscle inflammation, wherein the method comprises systemic administration to subject in a need thereof a therapeutically effective amount of at least one compound that disrupts a monocyte recruitment and/or macrophage expansion in response to muscle injury, wherein the method comprises decreasing Ly6C^{lo} macrophage expansion.

In certain embodiments, the at least one compound inhibits a colony stimulating factor-1 receptor (CSF-1R) signaling. In certain embodiments, the at least one compound is a CSF-1R inhibitor. In certain embodiments, the at least one compound is a colony stimulating factor-1 (CSF-1) inhibitor. In certain embodiments, the at least one compound is an inhibitor that disrupts CSF-1R-CSF1 binding.

In one aspect, provided herein is a method for treating chronic muscle inflammation, wherein the method comprises systemic administration to subject in a need thereof a therapeutically effective amount of at least one compound that disrupts a monocyte recruitment and disrupts macrophage expansion in response to muscle injury. In certain embodiments, the method comprises one or more of blocking recruitment of blood Ly6C^{hi} inflammatory monocyte by injured muscle, decreasing Ly6C^{hi} macrophage infiltration, and decreasing Ly6C^{lo} macrophage expansion.

In certain embodiments, the method comprises administering to subject in a need thereof a therapeutically effective amount of at least one compound that inhibits CCR2 signaling and at least one compound that inhibits CSF-1R signaling.

In certain embodiments, the at least one compound is a small molecule inhibitor. In certain embodiments, the at least one compound is a small molecule inhibitor which targets CCR2 signaling. In certain embodiments, the at least one compound is a small molecule inhibitor which targets CSF1-R signaling. In certain embodiments, the at least one compound is a peptide inhibitor. In certain embodiments, the at least one compound is a peptide inhibitor which targets CCR2 signaling. In certain embodiments, the at least one compound is a peptide inhibitor which targets CSF1-R signaling. In certain embodiments, the at least one compound is a protein inhibitor. In certain embodiments, the at least one compound is a protein inhibitor which targets CCR2 signaling. In certain embodiments, the at least one compound is a protein inhibitor which targets CSF1-R signaling. In certain embodiments, the at least one compound is an antibody. In certain embodiments, the at least one compound is a neutralizing antibody. In certain embodiments, the at least one compound is a neutralizing antibody targeting CCR2 and CSF-1R. In certain embodiments, the at least one compound is a neutralizing antibody targeting CCL2 and CSF-1. In certain embodiments, the at least one compound is a neutralizing antibody targeting CCL2and IL-34. In certain embodiments, the at least one compound is a nucleic acid molecule inhibitor (e.g., gene editing targeting the gene of Ccr2^{-/-} and Csflr). In certain embodiments, the at least one compound is a small interfering RNA (siRNA). In certain embodiments, the at least one compound is a small interfering RNA (siRNA) targeting mRNA of Cer2 and Csflr. In certain embodiments, the at least one compound is a small interfering RNA (siRNA) targeting mRNA Ccl2, Csfl, and Il34.

Exemplary CCR2 inhibitors include, without limitation: PF-04136309, TAK-652, RS504393, MK0812, PF-04634817, or INCB3344. See also, Sugiyama, S., et al., Identification of effective CCR2 inhibitors for cancer therapy using humanized mice, The Journal of Biochemistry, 2024, 175(2): 195-204; Tu, M.M., et al. Inhibition of the CCL2 receptor, CCR2, enhances tumor response to immune checkpoint therapy. Commun Biol 3, 720 (2020), which are incorporated herein by reference in their entireties.

Exemplary CSF-1 inhibitors include, without limitation: PLX3397, PLX5622, BLZ945, or PLX73086. See also, Cannarile, M. A., et al., Colony-stimulating factor 1 receptor (CSF1R) inhibitors in cancer therapy, Journal for Immuno Therapy of Cancer 2017, 5:53; Wen, J., et al., CSFIR inhibitors are emerging immunotherapeutic drugs for cancer treatment, European Journal of Medicinal Chemistry, 2023, 245(1); King, N. J. C., et al., Putting PLX 5622 into perspective: microglia in central nervous system viral infection. Neural Regeneration Research. 2023;18(6):1269; cancer. gov/publications/dictionaries/cancer-drug/def/sotuletinib, which are all incorporated herein by reference in their entirety.

For each of the at least one compound, i.e., agonist and antagonist, compositions thereof are provided herein. In certain embodiment, provided herein are compositions comprising at least one compound which composition additionally includes a pharmaceutically acceptable carrier. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In certain embodiments, the at least one compound is a nucleic acid molecule (optionally small interfering RNA (siRNA)), wherein the nucleic acid molecule is encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more siRNA to one or more target cells (e.g., muscle). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of an agent (e.g., nucleic acid molecule, siRNA) to a target cell. Useful lipid nanoparticles for and agent comprise a cationic lipid to encapsulate and/or enhance the delivery of the agent into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27(4):710-728). In some embodiments, LNP comprises a cationic lipids (i.e. N-[1-(2,3- dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, US9670152B2, and U.S. Pat. No. 8,853,377B2, which are incorporated by reference.

In one aspect provided herein is a regimen for treating chronic muscle inflammation, the regimen comprising administering to subject in a need thereof a therapeutically effective amount of at least one compound that disrupts a monocyte recruitment in response to muscle injury and at least one compound that disrupts macrophage expansion in response to muscle injury.

In one aspect provided herein is a regimen for treating chronic muscle inflammation, the regimen comprising administering to subject in a need thereof a therapeutically effective amount of at least one compound that disrupts a monocyte recruitment in response to muscle injury and at least one compound that disrupts macrophage expansion in response to muscle injury, wherein the regimen comprises administering of the at least one compound that disrupts a monocyte recruitment and at least one compound that disrupts macrophage expansion simultaneously.

In certain embodiments, the at least one compound that disrupts a monocyte recruitment and at least one compound that disrupts macrophage expansion are administered sequentially.

In certain embodiments, the regimen comprises first administering the at least one compound that disrupts a monocyte recruitment, followed by administering at least one compound that disrupts macrophage expansion. In certain embodiments, the at least one compound inhibits C-C motif chemokine receptor 2 (CCR2) signaling. In certain embodiments, the at least one compound inhibits a colony stimulating factor-1 receptor (CSF-1R) signaling. In certain embodiments, the regimen comprises first administering the at least one compound inhibits C-C motif chemokine receptor 2 (CCR2) signaling followed by administering the at least one compound inhibits a colony stimulating factor-1 receptor (CSF-1R) signaling.

In certain embodiments, the compound is administered systemically. Routes of administration include, but are not limited to, intradermal, intramuscular, intraperitoneal, intravenous, subcutaneous, intranasal, epidural, and oral routes. The at least one compound may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In certain embodiments, the at least one compound is administered orally. In certain embodiments, the at least one compound is administered by subcutaneous injection. In certain embodiments, the at least one compound is administered by intravenous injection. In certain embodiments, the compound is administered to a subject diagnosed with chronic muscle inflammation. In certain embodiments, the compound is administered to a subject diagnosed with muscular dystrophy. In certain embodiments, the compound is administered to a subject diagnosed with Duchenne Muscular Dystrophy (DMD). In certain embodiments, the compound is administered to a subject at risk for chronic muscle inflammation (e.g., genetically predisposed to chronic muscle inflammation). In certain embodiments, the compound is administered to a subject at risk for muscular dystrophy (e.g., genetically predisposed to muscular dystrophy). In certain embodiments, the compound is administered to a subject at risk for Duchenne Muscular Dystrophy (DMD) (e.g., genetically predisposed to DMD).

As described above, the terms “increase” “decrease” “reduce” “ameliorate” “improve” “delay” or any grammatical variation thereof, or any similar terms indication a change, means a variation of about 5000 fold, about 4000 fold, about 2000 fold, about 1000 fold, about 500 fold, about 200 fold, about 100 fold, 5 fold, about 2 fold, about 1 fold, or values therebetween, or about 90%, about 80%, about 70%, about 60%, about 50%, about 40%, about 30%, about 20%, about 10%, about 5% compared to the corresponding reference (e.g., untreated control or a subject in normal condition without Duchenne Muscular Dystrophy or chronic muscle inflammation, unless otherwise specified.

As used herein, the term “administration” or any grammatical variations thereof refers to delivery of composition described herein to a subject.

The term “fragment” is intended a molecule consisting of only a part of the intact full-length polypeptide sequence and structure. The fragment can include a C terminal deletion, an N terminal deletion, and/or an internal deletion of the native polypeptide. In one embodiment, the fragment includes an N-terminal deletion of up to 5, 10, 15, 20, 25, 30, 35, 40 or 45 amino acids. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 50 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 7 amino acids in length, and may be up to about 700 amino acids. Examples of suitable fragments are described herein. By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Multiple sequence alignment programs are available for nucleic acid sequences. Examples of such programs include, “Clustal Omega”, “Clustal W”, “MUSCLE”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 10.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 10.1, herein incorporated by reference. Sequence alignment programs are also available for amino acid sequences, e.g., the “Clustal Omega”, “Clustal X”, “MUSCLE”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

As described above, the term “about” when used to modify a numerical value means a variation of ±10%, (±10%, e.g., ±1, ±2, +3, +4, ±5, ±6, ±7, ±8, ±9, ±10, or values therebetween) from the reference given, unless otherwise specified. For example, “about” 500 μM includes ±50 (i.e., 450-550, which includes the integers therebetween). For other values, particularly when reference is to a percentage (e.g., 90%), the term “about” is inclusive of all values within the range including both the integer and fractions.

In certain instances, the term “E+#” or the term “e+#” is used to reference an exponent. For example, “5E 10” or “5e 10” is 5×10¹⁰. These terms may be used interchangeably.

As used throughout this specification and the claims, the recitations of numerical ranges by endpoints include all numbers subsumed within that range (e.g., 1 to 5 includes 1, 1.5, 2, 2.75, 3, 3.80, 4, 5, etc.). Herein, “up to” a number (for example, up to 50) includes the number (for example, 50). The term “in the range” or “within a range” (and similar statements) includes the endpoints of the stated range.

“Upregulate” and “upregulation”, as used herein, refer to an elevation in the level of expression of a product of one or more genes in a cell or the cells of a tissue or organ.

As used herein, the term “agonist” refers to a compound that in combination with a receptor can produce a cellular response. An agonist may be a ligand that directly binds to the receptor. Alternatively, an agonist may combine with a receptor indirectly by for example (a) forming a complex with another molecule that directly binds to the receptor, or (b) otherwise resulting in the modification of another compound so that the other compound directly binds to the receptor.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider.

“Sample” as used herein means any biological fluid or tissue that contains cells or tissue, including blood cells, fibroblasts, and skeletal muscle. In one embodiment, the sample is whole blood. In another embodiment, the sample is peripheral blood mononuclear cells (PBMC). Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

The term “derived from” is used to identify the original source of a molecule (e.g., murine or human) but is not meant to limit the method by which the molecule is made which can be, for example, by chemical synthesis or recombinant means.

As used herein, the term “a therapeutically effective amount” refers an amount sufficient to achieve the intended purpose. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

In certain embodiments, for a small molecule inhibitor, a peptide inhibitor, protein or peptide antagonist, e.g., antibody, antibody fragment or recombinant protein or peptide, the effective amount can be about 0.01 to 25 mg antibody/injection. In one embodiment, the effective amount is 0.01 to 10 mg antibody/injection. In another embodiment, the effective amount is 0.01 to 1 mg antibody/injection. In another embodiment, the effective amount is 0.01 to 0.10 mg antibody/injection. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg antibody/injection. Still other doses falling within these ranges are expected to be useful. In one embodiment an effective amount for the nucleic acid and/or protein inhibitor of composition (a) includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 1 to 20 mg/kg body weight. Still other doses falling within these ranges are expected to be useful.

In certain embodiments, an effective amount may be determined based on an animal model, rather than a human patient.

In one embodiment, the effective amount of the CCR2 inhibitor is an amount ranging from about 0.01 mg/ml to about 10 mg/ml, including all amounts therebetween and end points. In one embodiment, the effective amount of the CCR2 inhibitor is about 0.1 mg/ml to about 5 mg/ml, including all amounts therebetween and end points. In another embodiment, the effective amount of the CCR2 inhibitor is about 0.3 mg/ml to about 1.0 mg/ml, including all amounts therebetween and end points. In another embodiment, the effective amount of the CCR2 inhibitor is about 0.3 mg/ml. In another embodiment, the effective amount of the CCR2 inhibitor is about 0.4 mg/ml. In another embodiment, the effective amount of the CCR2 inhibitor is about 0.5 mg/ml. In another embodiment, the effective amount of the CCR2 inhibitor is about 0.6 mg/ml. In another embodiment, the effective amount of the CCR2 inhibitor is about 0.7 mg/ml. In another embodiment, the effective amount of the CCR2 inhibitor is about 0.8 mg/ml. In another embodiment, the effective amount of the CCR2 inhibitor is about 0.9 mg/ml. In another embodiment, the effective amount of the CCR2 inhibitor is about 1.0 mg/ml.

In one embodiment, the effective amount of the CCR2 inhibitor is an amount ranging from about 1 μM to about 2 mM, including all amounts therebetween and end points. In one embodiment, the effective amount of the CCR2 inhibitor is about 10 μM to about 100 μM, including all amounts therebetween and end points. In another embodiment, the effective amount of the CCR2 inhibitor is about 5μM. In another embodiment, the effective amount of the CCR2 inhibitor is about 10 μM. In another embodiment, the effective amount of the CCR2 inhibitor is about 20 μM. In another embodiment, the effective amount of the CCR2 inhibitor is about 50 μM. In another embodiment, the effective amount of the CCR2 inhibitor is about 100μM. In another embodiment, the effective amount of the CCR2 inhibitor is about 200 μM. In another embodiment, the effective amount of the CCR2 inhibitor is about 300 μM. In another embodiment, the effective amount of the CCR2 inhibitor is about 400 μM. In another embodiment, the effective amount of the CCR2 inhibitor is about 500 μM. In another embodiment, the effective amount of the CCR2 inhibitor is about 600 μM. In another embodiment, the effective amount of the CCR2 inhibitor is about 700 μM. In another embodiment, the effective amount of the CCR2 inhibitor is about 800 μM. In another embodiment, the effective amount of the CCR2 inhibitor is about 900 μM. In another embodiment, the effective amount of the CCR2 inhibitor is about 1 mM. In another embodiment, the effective amount of the CCR2 inhibitor is about 1.25 mM. In another embodiment, the effective amount of the CCR2 inhibitor about 1.5 mM. In another embodiment, the effective amount of the CCR2 inhibitor is about 1.75 mM. In another embodiment, the effective amount of the CCR2 inhibitor is about 2 mM.

In one embodiment, the effective amount of the CSF-1R inhibitor is an amount ranging from about 0.01 mg/ml to about 10 mg/ml, including all amounts therebetween and end points. In one embodiment, the effective amount of the CSF-1R inhibitor is about 0.1 mg/ml to about 5 mg/ml, including all amounts therebetween and end points. In another embodiment, the effective amount of the CSF-1R inhibitor is about 0.3 mg/ml to about 1.0 mg/ml, including all amounts therebetween and end points. In another embodiment, the effective amount of the CSF-1R inhibitor is about 0.3 mg/ml. In another embodiment, the effective amount of the CSF-1R inhibitor is about 0.4 mg/ml. In another embodiment, the effective amount of the CSF-1R inhibitor is about 0.5 mg/ml. In another embodiment, the effective amount of the CSF-1R inhibitor is about 0.6 mg/ml. In another embodiment, the effective amount of the CSF-1R inhibitor is about 0.7 mg/ml. In another embodiment, the effective amount of the CSF-1R inhibitor is about 0.8 mg/ml. In another embodiment, the effective amount of the CSF-1R inhibitor is about 0.9 mg/ml. In another embodiment, the effective amount of the CSF-1R inhibitor is about 1.0 mg/ml.

In one embodiment, the effective amount of the CSF-1R inhibitor is an amount ranging from about 1 μM to about 2 mM, including all amounts therebetween and end points. In one embodiment, the effective amount of the CSF-1R inhibitor is about 10 μM to about 100 μM, including all amounts therebetween and end points. In another embodiment, the effective amount of the CSF-1R inhibitor is about 5 μM. In another embodiment, the effective amount of the CSF-1R inhibitor is about 10 μM. In another embodiment, the effective amount of the CSF-1R inhibitor is about 20 μM. In another embodiment, the effective amount of the CSF-1R inhibitor is about 50 μM. In another embodiment, the effective amount of the CSF-1R inhibitor t is about 100 μM. In another embodiment, the effective amount of the CSF-1R inhibitor is about 200 μM. In another embodiment, the effective amount of the CSF-1R inhibitor is about 300 μM. In another embodiment, the effective amount of the CSF-1R inhibitor is about 400 μM. In another embodiment, the effective amount of the CSF-1R inhibitor is about 500 μM. In another embodiment, the effective amount of the CSF-1R inhibitor is about 600 μM. In another embodiment, the effective amount of the CSF-1R inhibitor is about 700 μM. In another embodiment, the effective amount of the CSF- 1R inhibitor is about 800 μM. In another embodiment, the effective amount of the CSF-1R inhibitor is about 900 μM. In another embodiment, the effective amount of the CSF-1R inhibitor is about 1 mM. In another embodiment, the effective amount of the CSF- 1R inhibitor is about 1.25 mM. In another embodiment, the effective amount of the CSF- 1R inhibitor about 1.5 mM. In another embodiment, the effective amount of the CSF-1R inhibitor is about 1.75 mM. In another embodiment, the effective amount of the CSF-1R inhibitor is about 2 mM.

As used herein, “disease”, “disorder” and “condition” are used interchangeably, to indicate an abnormal state in a subject.

“Treatment,” as used herein, refers to the application or administration of an agent, or pharmaceutical composition containing the agent, to a subject, isolated tissue, isolated cells or cell line from a subject, where the subject has a disease or condition, or a predisposition toward development of a disease or condition, where the purpose is to cure, heal, alleviate, relieve, alter, remedy, ameliorate, improve, or affect the disease or condition and/or any associated symptoms of the disease or condition.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are provided to illustrate certain aspects of the claimed invention. The invention is not limited to these examples.

### Example 1

### Abstract

Infiltrating macrophages contribute to muscle dystrophic changes in Duchenne muscular dystrophy (DMD). In a DMD mouse model, mdx^{5cv} mice, CC chemokine receptor type 2 (CCR2) deficiency diminishes Ly6C^{hi} macrophage infiltration by blocking blood Ly6C^{hi} inflammatory monocyte recruitment. This is accompanied by transient improvement of muscle damage, fibrosis, and regeneration. The benefit, however, is lost after the expansion of intramuscular Ly6C^{lo} macrophages. To address the mechanisms underlying the Ly6C^{lo} macrophage expansion, we compared mdx^{5cv}/Nur77^{-/-} and mdx^{5cv}/Ccr2^{-/-}/Nur7^{-/-} mice with mdx^{5cv} and mdx^{5cv}/Ccr2^{-/-} mice, respectively, and found no evidence to suggest Ly6C^{lo} monocyte recruitment by dystrophic muscles. Single-cell RNA sequencing analysis and Flt3^{cre}/Rosa26^{LSL-YFP}-based lineage tracing of macrophage origins demonstrated the expansion and pathogenic activation of muscle resident macrophages in CCR2-deficient mdx^{5cv} mice. The expansion was associated with increased cell proliferation, which appeared induced by colony stimulating factor-1 (CSF-1) derived from fibro/adipogenic progenitors (FAPs). Our study establishes a pathogenic role for skeletal muscle resident macrophages and supports a regulatory role of FAPs in stimulating the expansion of resident macrophage in the DMD mouse model when the inflammatory macrophage infiltration is inhibited.

### Introduction

Duchenne muscular dystrophy (DMD) is the most common genetic muscle disease caused by a defective dystrophin gene on X chromosome (1, 2), which leads to muscle membrane instability, muscle fiber necrosis, chronic muscle inflammation, progressive muscle fibrosis, muscle dysfunction, and premature death from respiratory and cardiac muscle weakness (3-6). Studies by our group and others have demonstrated that suppressing muscle inflammation and fibrosis represents a viable therapeutic approach for DMD (7-13). It may not only improve muscle function and dystrophy phenotype but also enhance gene and stem cell delivery and engraftment efficiency (11). The most commonly used animal model for studying DMD is mdx mice. Muscle necrosis and inflammation in skeletal muscle of mdx mice starts around 3 weeks of age and peaks and persists into 2 to 3 months of age. After that, the inflammation recedes spontaneously in limb muscles. Diaphragm, the main respiratory muscle, undergoes persistent inflammation and progressive fibrosis, which is accompanied by impaired respiratory function, mimicking human DMD (6, 8, 14, 15).

Chronic muscle inflammation associated with DMD and mdx is predominated by inflammatory monocyte and macrophage infiltration. Infiltrating macrophages are largely pathogenic in this setting, which can promote muscle damage and fibrosis (8, 9). Murine blood monocytes consist of two principal subsets, CX3CR1^{lo}CCR2⁺Ly6C^{hi} and CX3CR1^{hi}CCR2⁻Ly6C^{lo} cells (16). CC chemokine receptor 2 (CCR2) and its ligands play a critical role in mediating tissue recruitment of Ly6C^{hi} monocytes (17-20). We and others have shown that CCR2 deficiency blocks the muscle recruitment of Ly6C^{hi} monocytes and diminishes intramuscular Ly6C^{hi} macrophages in mdx and mdx^{5cv} mice, resulting in decreased muscle damage, reduced muscle fibrosis, and improved muscle regeneration (8, 9). However, CCR2 deficiency does not provide a sustained benefit to mdx^{5cv} diaphragm, as dystrophic changes worsen after 3 months of age, following the expansion of intramuscular Ly6C^{lo} macrophages, despite the persistent reduction of intramuscular Ly6C^{hi} macrophages (9). Therefore, targeting Ly6C^{hi} macrophages alone is not sufficient, and Ly6C^{lo} macrophages may also play pathogenic roles in muscular dystrophy. Understanding the origins and functional regulations of intramuscular Ly6C^{lo} macrophages is important.

In the present study, we have addressed the mechanisms underlying the expansion of intramuscular Ly6C^{lo} macrophage in mdx^{5cv}/Ccr2^{-/-} mice. By comparing mdx^{5cv}/Nur77^{-/-} and mdx^{5cv}/Ccr2^{-/-}/Nur7^{-/-} mice with mdx^{5cv} and mdx^{5cv}/Ccr2^{-/-} mice, respectively, we found that the lack of blood Ly6C^{lo} monocytes did not affect the density of intramuscular Ly6C^{lo} macrophages, inferring no Ly6C^{lo} monocytes recruitment by dystrophic muscles. Combining single-cell RNA sequencing analysis (scRNAseq) and Flt3^{cre}/Rosa26^{LSL-YFP}-based lineage tracing to identify resident macrophages originated from hematopoietic stem cells (HSCs) (YFP+) and non-HSCs (YFP), we found the expansion of muscle resident macrophages in CCR2-deficient mdx^{5cv} mice, especially those of embryo-derived, non-HSC origins. The expansion was contributed by increased cell proliferation, which were likely stimulated by colony stimulating factor 1 (CSF-1) that was predominantly produced by fibro/adipogenic progenitors (FAPs) in dystrophic muscles. In addition, muscle resident macrophages in mdx^{5cv}/Ccr2^{-/-} mice underwent pathogenic activation, similar to the intramuscular macrophages in mdx^{5cv} mice. Our study establishes a pathogenic role for skeletal muscle resident macrophages in the DMD mouse model when the inflammatory macrophage infiltration is inhibited.

### Example 2

### Results

A. Nur77 deficiency diminishes blood Ly6C^{lo} monocytes but does not affect the density of intramuscular Ly6C^{lo} macrophages in dystrophic muscles.

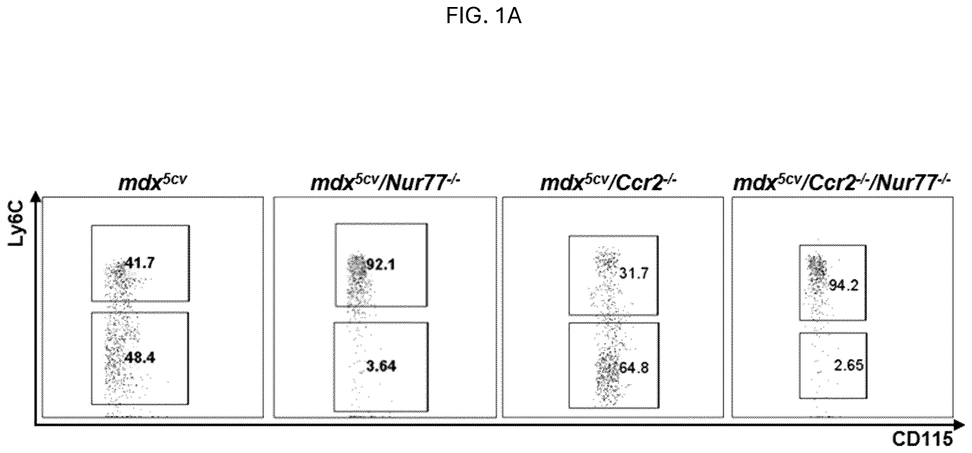

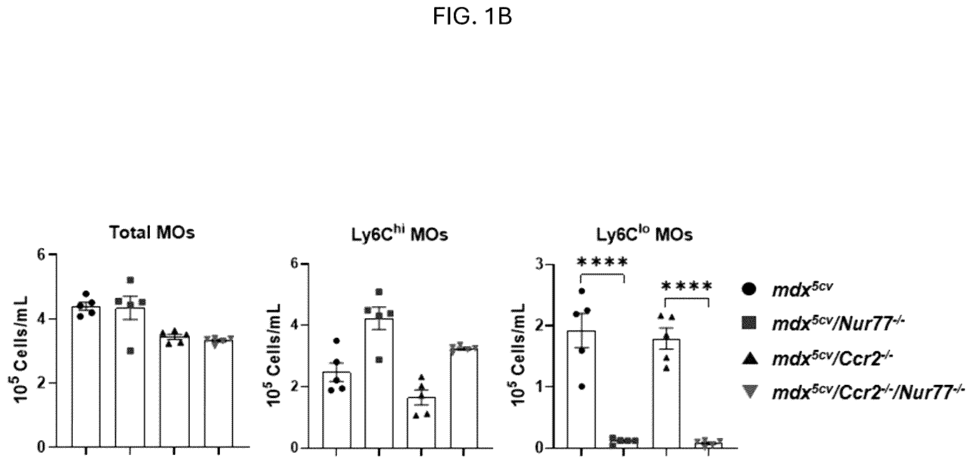

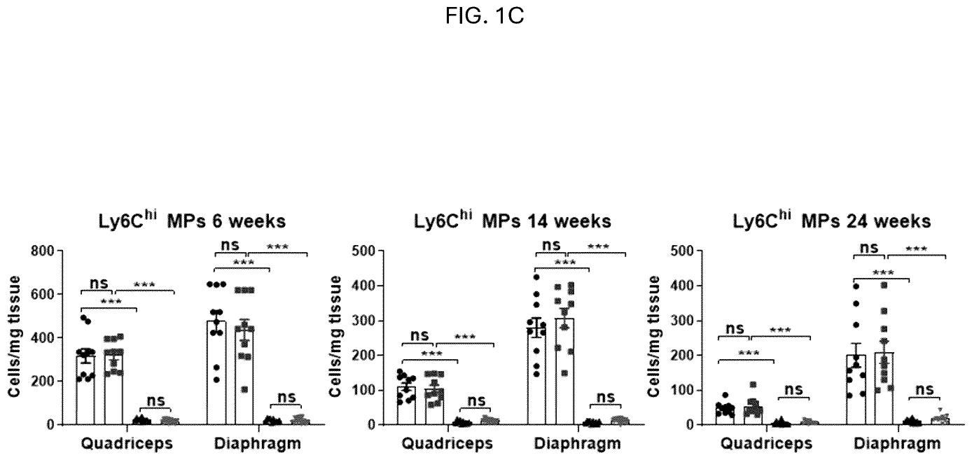

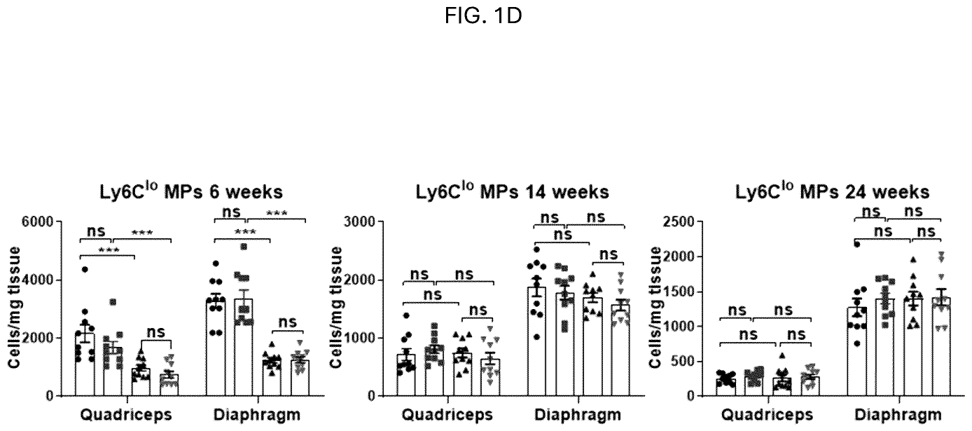

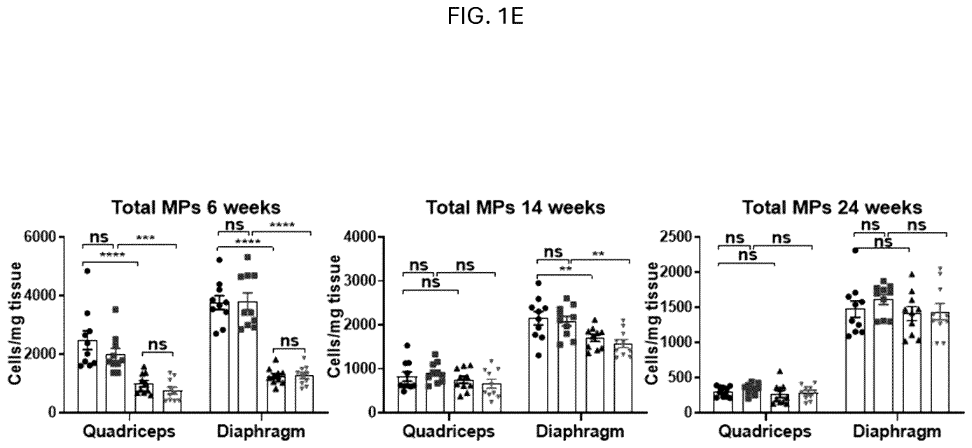

Intramuscular Ly6C^{lo} macrophages in dystrophic muscles can be potentially derived from three origins: 1) Ly6C^{hi} blood monocytes-derived Ly6C^{hi} macrophages which undergo Ly6C^{hi}-to-Ly6C^{lo} phenotype switch within injured muscle (21, 22), 2) recruitment of blood Ly6C^{lo} monocytes which then differentiate into Ly6C^{lo} macrophages, and 3) pre-existing muscle resident macrophages which are Ly6C^{lo} (23, 24). Since the recruitment of Ly6C^{hi} monocyte is largely blocked by CCR2 deficiency in mdx^{5cv}/Ccr2^{-/-} mice (9), the Ly6C^{hi}-to-Ly6C^{lo} phenotype switch contributes minimally to intramuscular Ly6C^{lo} macrophages in these mice. To address whether Ly6C^{lo} monocytes are recruited to compensate for the lack of Ly6C^{hi} monocyte recruitment caused by CCR2 deficiency, we generated mdx^{5cv}/Nur77^{-/-} and mdx^{5cv}/Ccr2^{-/-}/Nur7^{-/-} mice to compare with mdx^{5cv} and mdx^{5cv}/Ccr2^{-/-} mice, respectively. Nur77 is a nuclear factor that is essential for the differentiation of Ly6C^{lo} monocytes from Ly6C^{hi} monocytes (25). As expected, blood Ly6C^{lo} monocytes were diminished in mdx^{5cv}/Nur77^{-/-} and mdx^{5cv}/Ccr2^{-/-}/Nur7^{-/-} mice gauged by flow cytometry analysis (FACS), but the total number of monocytes were unchanged as the number of Ly6C^{hi} monocytes was increased (FIGS. 1A-B).

FIGS. 1A-1E show that Nur77 deficiency diminishes blood Ly6C^{lo} monocytes but does not affect the density of Ly6C^{hi} or Ly6C^{lo} macrophages in mdx^{5cv} or mdx^{5cv}/Ccr2^{-/-} quadriceps and diaphragm. FIG. 1A shows represented dot plots of FACS analysis showing Ly6C^{hi} and Ly6C^{lo} blood monocytes (MOs) in mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age. FIG. 1B shows bar graph showing densities of blood MOs quantified from FACS analysis. N=5 mice/group. ****p<0.0001. FIG. 1C to E show bar graph showing densities of intramuscular Ly6C^{hi} (C), Ly6C^{lo} (FIG. 1D), and total (FIG. 1E) macrophages (MPs) quantified by flow cytometry in the diaphragm and quadriceps of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at ages 6 weeks, 14 weeks, and 24 weeks. N=10 mice/group. **p<0.01; ***p<0.001; ****p<0.0001; ns: no significance.

FACS using single cell suspensions of quadriceps and diaphragm from mdx^{5cv} mdx^{5cv}/Nur77^{-/-}, mdx^{5cv}/Ccr2^{-/-}, mdx^{5cv}/Ccr2^{-/-}/Nur7^{-/-} mice at 6, 14, and 24 weeks of ages further showed that, while CCR2 deficiency diminished Ly6C^{hi} macrophages at all the stages due to blockage of blood Ly6C^{hi} monocyte recruitment (FIG. 1C), it only reduced Ly6C^{lo} macrophages at 6 weeks but not 14 weeks or 24 weeks (FIG. 1D), consistent with our previous findings (9).

Similarly, the total number of intramuscular macrophages decreased significantly at 6 weeks but not 14 or 24 weeks (FIG. 1E), as Ly6C^{lo} macrophages expanded and were more abundant than Ly6C^{hi} macrophages (FIGS. 1C-D). Importantly, Nur77 deficiency did not affect the density of intramuscular Ly6C^{hi} or Ly6C^{lo} macrophages in either mdx^{5cv}/Nur77^{-/-} or mdx^{5cv}/Ccr2^{-/-}/Nur7^{-/-} mice at any stages (FIGS. 1C-D), suggesting that blood Ly6C^{lo} monocytes are unlikely recruited to dystrophic muscles to contribute to the expansion of intramuscular Ly6C^{lo} macrophages in mdx^{5cv}/Ccr2^{-/-} mice.
B. Ly6C^{lo} resident macrophages expand in mdx^{5cv}/Ccr2^{-/-} quadriceps and diaphragm.

We next addressed whether muscle resident macrophages expanded to contribute to the intramuscular Ly6C^{lo} macrophages in mdx^{5cv}/Ccr2^{-/-} mice. Skeletal muscle resident macrophages reside in interstitial tissue and they are CD45⁺F4/80⁺CD64⁺Ly6C^{lo} cells (24). Adult skeletal muscle resident macrophages mostly arise from fetal monocytes and adult bone marrow HSCs, with a small percentage from yolk-sac primitive macrophages (24). Besides late erythro-myeloid progenitors (EMPs), prenatal HSCs also contribute to fetal monocytes at the late stage of embryonic development. Fetal monocytes seed different tissues including skeletal muscle to differentiate into tissue resident macrophages. The embryo-derived tissue resident macrophages can persist into adulthood via self-renewal (24, 26, 27). After birth, CCR2⁺ blood monocytes gradually and partially replenish skeletal muscle resident macrophages (24). Therefore, in the steady state, skeletal muscle resident macrophages of the non-HSC origin are purely derived from embryo, while those from the HSC origin can be derived from either late-stage embryo or adult bone marrow. The origins of resident macrophages and the contribution of these cells to the intramuscular Ly6C^{lo} macrophages in dystrophic muscles remain largely unknown.

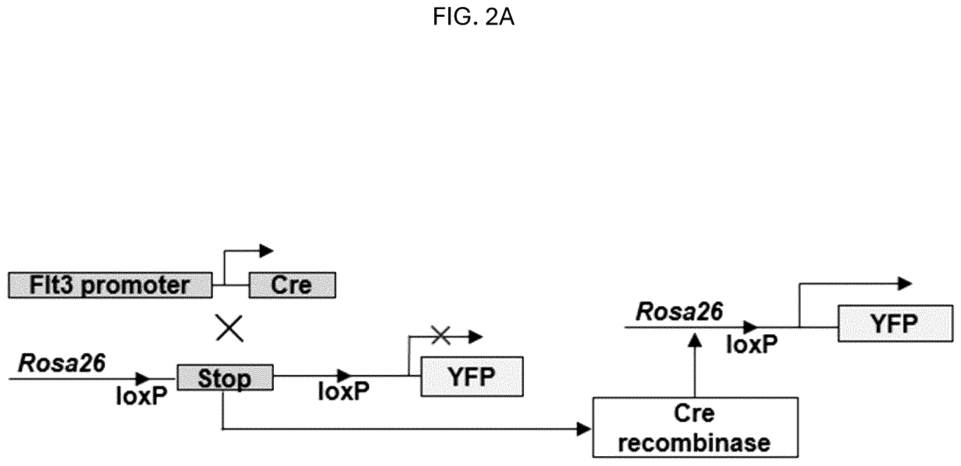

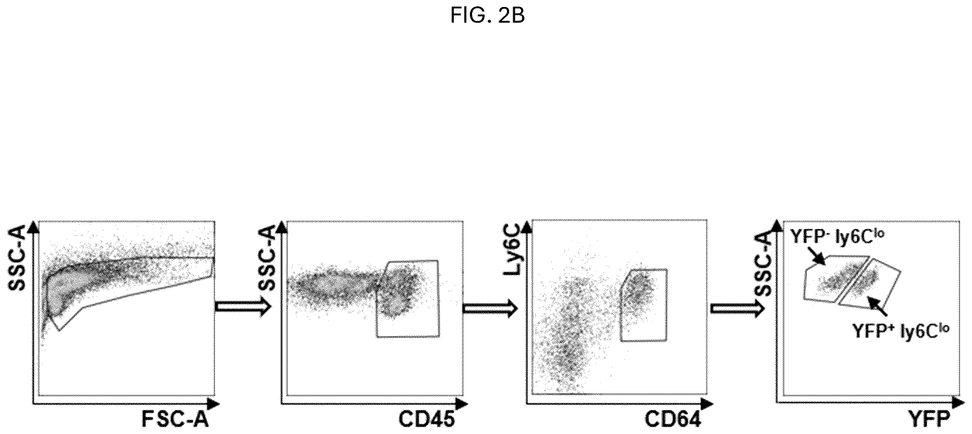

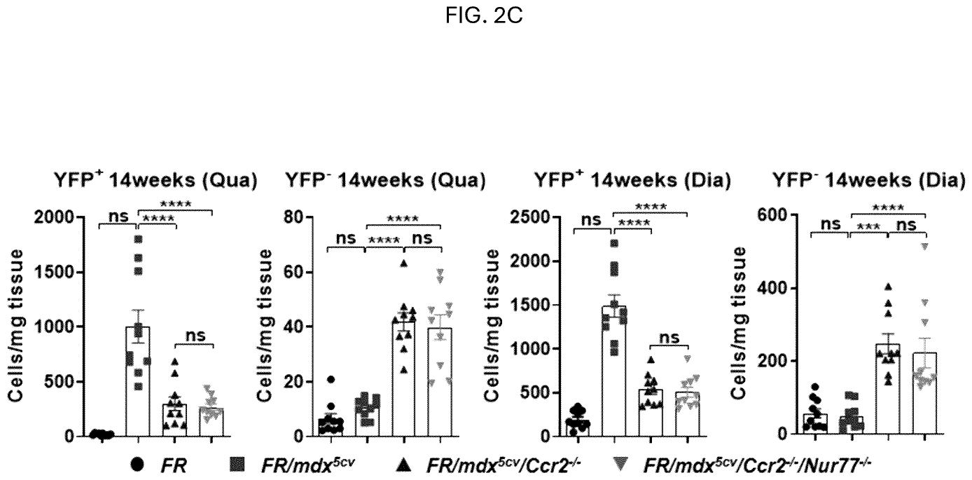

To address the possible expansion of intramuscular resident macrophage in mdx^{5cv}/Ccr2^{-/-} mice, we used the Flt3^{Cre}/Rosa26^{LSL-YFP}-based lineage tracing system which can distinguish HSC-derived macrophages from non-HSC-originated resident macrophages. HSCs transiently upregulate growth factor fms-like tyrosine kinase 3 (Flt3) when they differentiate into individual hematopoietic lineages including blood monocytes (28). Therefore, macrophages of HSC origin express YFP (YFP⁺) in Flt3^{Cre}/Rosa26^{LSL-YFP} mice, while resident macrophages of non-HSC origin are YFP (FIGS. 2A-B).

FIGS. 2A-2C show embryo-derived Ly6C^{lo} resident macrophages expand in mdx^{5cv}/Ccr2^{-/-} quadriceps and diaphragm. FIG. 2A shows scheme of Flt3^{Cre}/Rosa26^{LSL-YFP} lineage tracing. FIG. 2B shows dot plots showing gating strategy for FACS identification of YFP+(CD45⁺/CD64⁺/Ly6C^{lo} /YFP⁺) and YFP(CD45⁺/CD64⁺/Ly6C^{lo} /YFP⁻) Ly6C^{lo} macrophages with skeletal muscle single-cell suspensions. FIG. 2C shows bar graph showing densities of intramuscular YFP⁺ and YFP³¹ Ly6C^{lo} macrophages in the quadriceps (Qua) and diaphragm (Dia) of Flt3^{Cre}/Rosa26^{LSL-YFP} (FR), Flt3^{Cre}/Rosa26^{LSL-YFP}/mdx^{5cv} (FR/mdx^{5cv}), Flt3^{Cre}/Rosa26^{LSL-YFP}/mdx^{5cv}/Ccr2^{-/-} (FR/mdx^{5cv}/Ccr2^{-/-}) , and Flt3^{Cre}/Rosa26^{LSL-YFP}/mdx^{5cv}/Ccr2^{-/-}/ Nur77^{-/-} (FR/mdx^{5cv}/Ccr2^{-/-}/Nur7^{-/-}) mice at 14 weeks of age. N=10 mice/group. ***p<0.001; ****p<0.0001; ns: no significance.

Due to the incomplete recombination in the cre-lox system (85-90%), we adjusted YFP⁺ and YFP⁻ intramuscular macrophage cell density based on the recombination rate determined by the percentage of YFP⁺ monocytes in blood, as adult blood monocytes completely originate from HSCs and should be 100% YFP⁺. The Flt3^{Cre}/Rosa26^{LSL-YFP} mice were crossed to mdx^{5cv}, mdx^{5cv}/Ccr2^{-/-}, and mdx^{5cv}/Ccr2^{-/-}/Nur7^{-/-} mice for assessing the origins of intramuscular Ly6C^{lo} macrophages. The density of YFP⁺, but not YFP, Ly6C^{lo} macrophages was increased in quadriceps and diaphragm of Flt3^{cre}/Rosa26^{LSL-YFP}/mdx^{5cv} mice as compared to Flt3^{cre}/Rosa26^{LSL-YFP} controls at 14 weeks (FIG. 2C), indicating that the Ly6C^{lo} macrophages in mdx^{5cv} muscles were mainly from phenotype-switch of the HSC-originated, blood monocytes-derived Ly6C^{hi} macrophages. CCR2 deficiency significantly, but not completely, decreased the density of YFP⁺ Ly6C^{lo} macrophages, while significantly increased the density of YFP⁻ Ly6C^{lo} macrophages (FIG. 2C), suggesting that the embryo-derived resident macrophages of non-HSC origin expanded and the embryo-derived resident macrophages of HSC origin might also contribute to the intramuscular Ly6C^{lo} macrophages in mdx^{5cv}/Ccr2^{-/-} mice. Nur77 deficiency, however, did not affect either YFP⁻ or YFP⁺ macrophage cell density in Flt3^{cre}/Rosa26^{LSL-YFP}/mdx^{5cv}/Ccr2^{-/-}/ Nur77^{-/-} mice as compared with Flt3^{cre}/Rosa26^{LSL-YFP}/mdx^{5cv}/Ccr2^{-/-} controls (FIG. 2C). The findings further indicate that blood Ly6C^{lo} monocytes are not recruited to skeletal muscles of mdx^{5cv}/Ccr2^{-/-} mice. Therefore, the expansion of Ly6C^{lo} macrophages in mdx^{5cv}/Ccr2^{-/-} mice are resulted from resident macrophage expansion rather than blood Ly6C^{lo} monocyte recruitment.

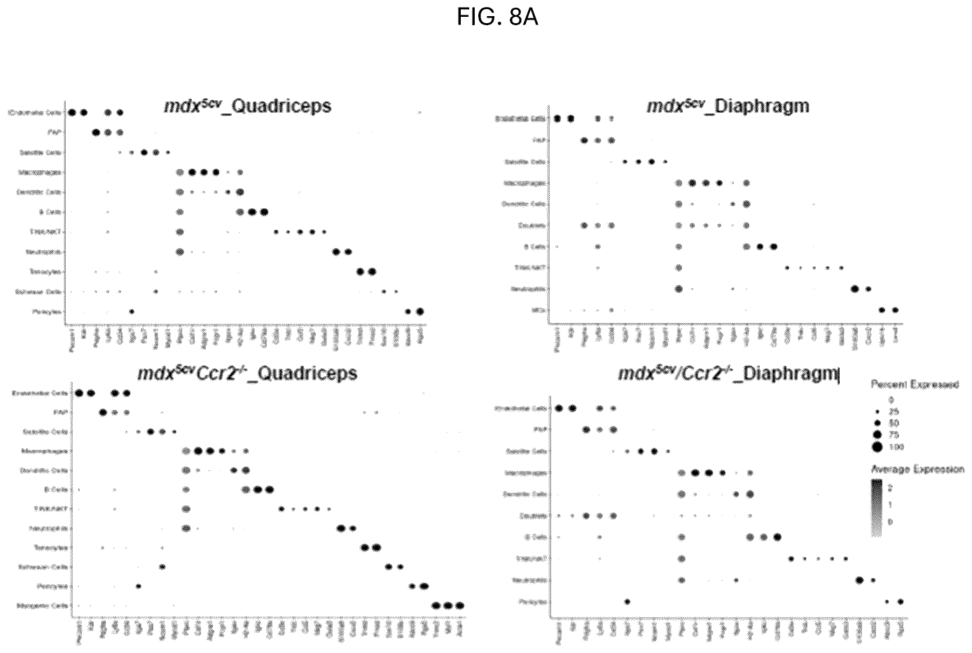

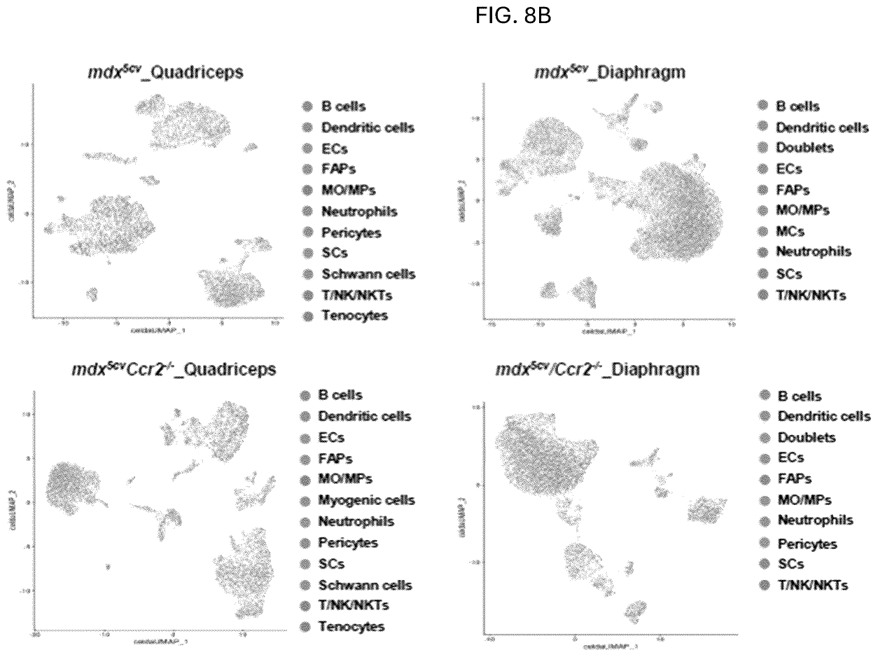

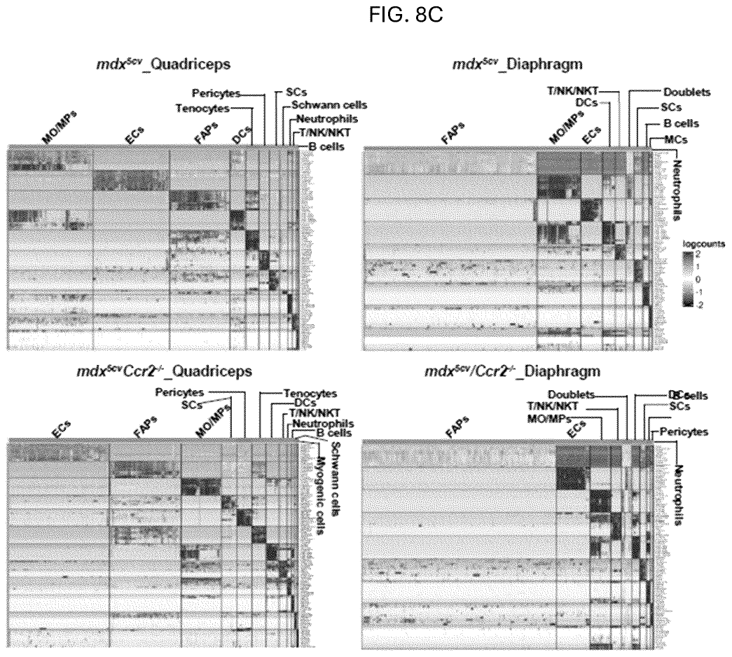

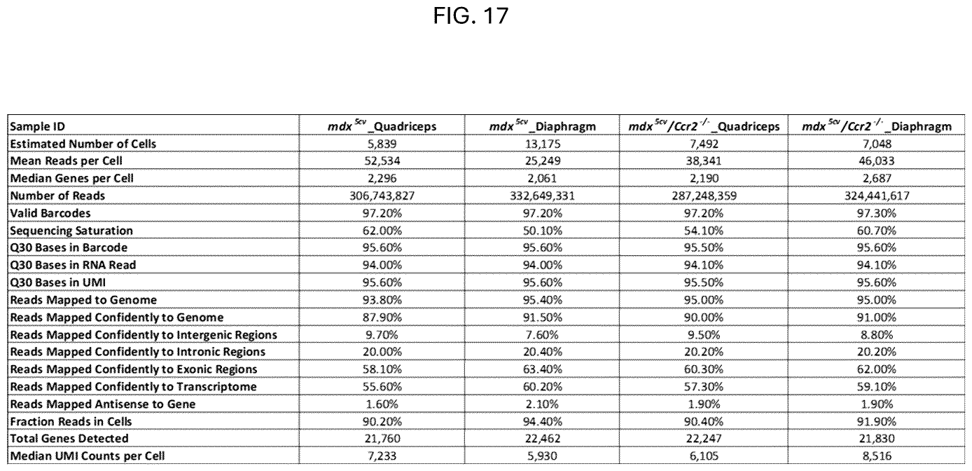

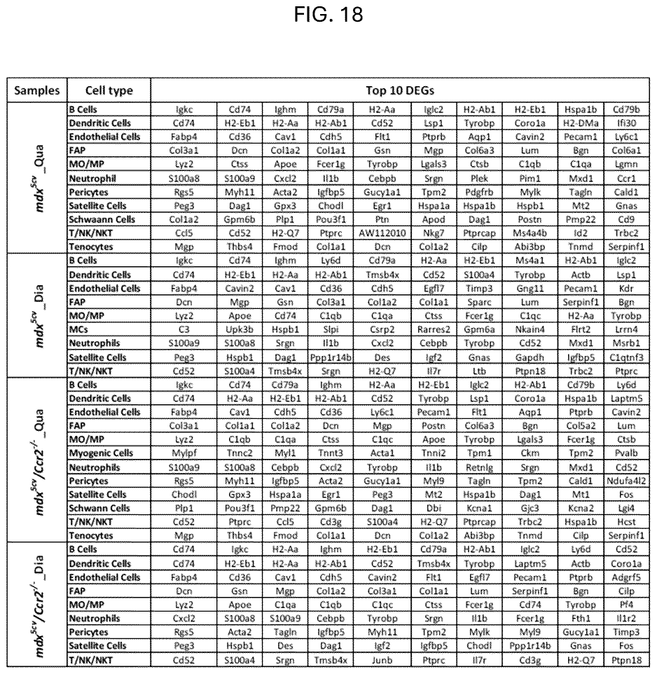

To further address the expansion of muscle resident macrophages in mdx^{5cv}/Ccr2^{-/-} mice, we performed scRNAseq using single cell suspensions prepared from quadriceps and diaphragm of mdx^{5cv}/Ccr2^{-/-} and mdx^{5cv} mice at 14 weeks of age. The single cell suspensions contain mononuclear cells but not polynuclear myofibers due to their large size. scRNAseq of the four muscle samples, each pooled from 5 male mice, were performed simultaneously (see Table in FIG. 17 for quality control). FIG. 17 shows a table describing scRNAseq Quality Control. By filtering out the cells of low quality, we obtained 21,591 genes from 4,752 cells of mdx^{5cv} quadriceps, 22,342 genes from 11,968 cells of mdx^{5cv} diaphragm, 22,099 genes from 6,154 cells of mdx^{5cv}/Ccr2^{-/-} quadriceps, and 21,651 genes from 6,014 cells of mdx^{5cv}/Ccr2^{-/-} diaphragm for analysis. Sequencing data were first analyzed using Uniform Mani-fold Approximation and Projection (UMAP) for dimension reduction to generate functionally enriched clusters in each sample. The identities of the generated clusters were determined by the expression of cell type-specific marker genes (FIG. 8A). FIG. 8A shows dot plots of single-cell RNA sequencing (scRNAseq) data showing the expression of marker genes by different cell types in each muscle sample. FIG. 8B shows uniform Mani-fold Approximation and Projection (UMAP) dimension reduction analysis of scRNAseq data showing different cell types in mdx^{5cv} and mdx^{5cv}/Ccr2^{-/-} quadriceps and diaphragm muscles. FIG. 8C shows heatmap depicting top 10 differentially expressed genes (DEGs) of individual cell types in each muscle sample. The names of the top 10 DEGs of each cell type are listed in FIG. 18. ECs: Endothelial cell, DCs: Dendritic Cell, MO/MPs: Monocyte/Macrophage, FAPs: Fibro/adipogenic progenitors, T: T cells, NK: Natural killer cells, NKT: invariant Natural Killer T Cells, MC: Mesothelial cells, SCs: Satellite cells.

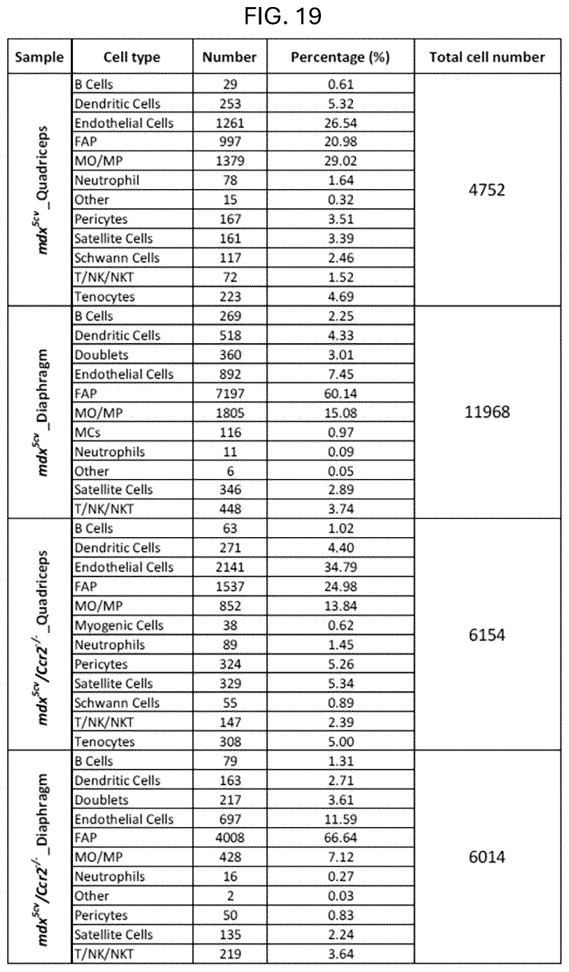

Multiple cell types, including macrophages, were identified in each sample (FIGS. 8B-C, and Tables in FIGS. 18 and 19). FIG. 18 shows a table describing top 10 differentially expressed genes by different intramuscular cell types in quadriceps (Qua) and diaphragm (Dia) muscles of mdx^{5cv} and mdx^{5cv}/Ccr2^{-/-} mice. FIG. 19 shows a table describing number and percentage of each cell type identified by scRNAseq in mdx^{5cv} and mdx^{5cv}/Ccr2^{-/-} quadriceps and diaphragm muscles.

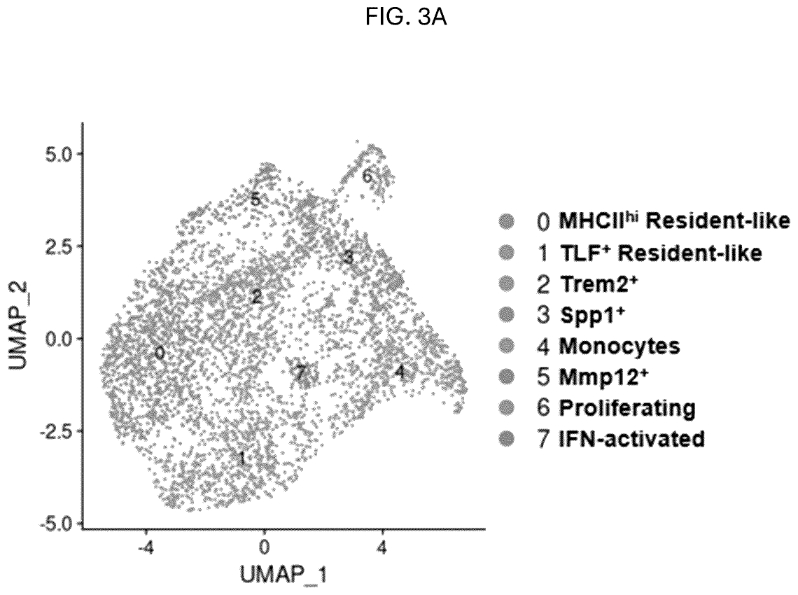

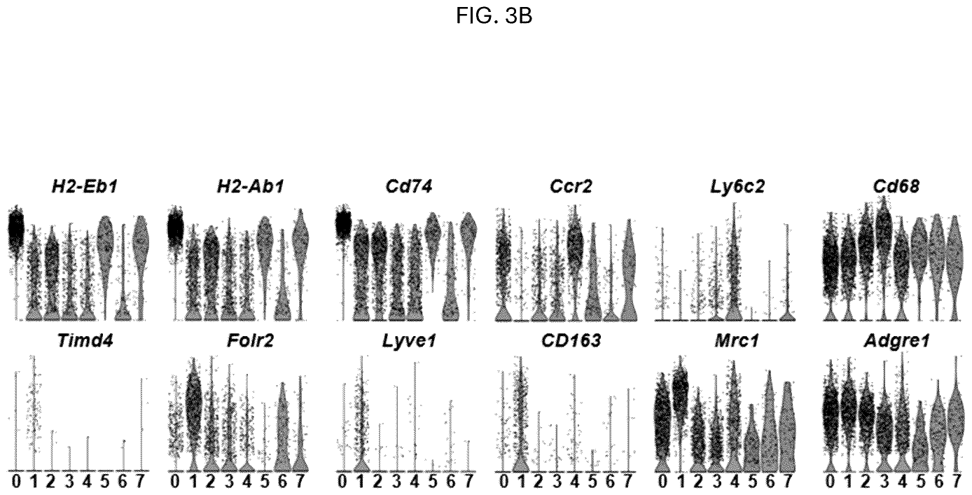

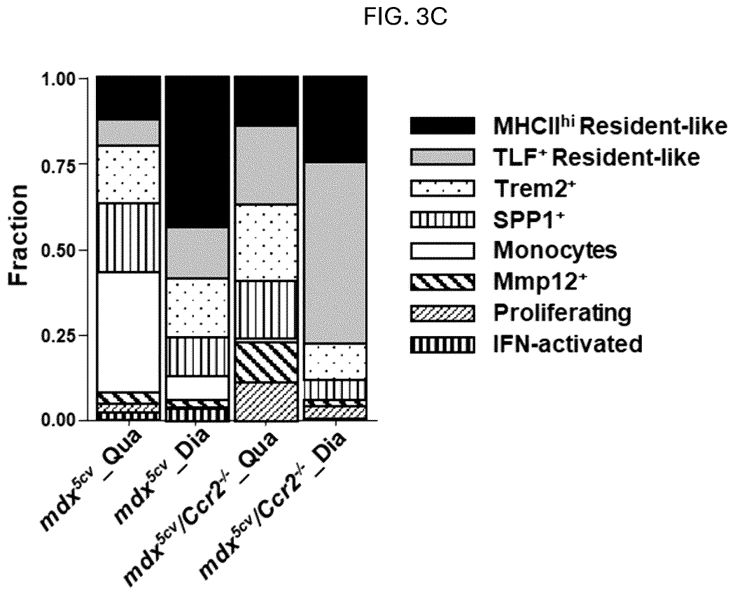

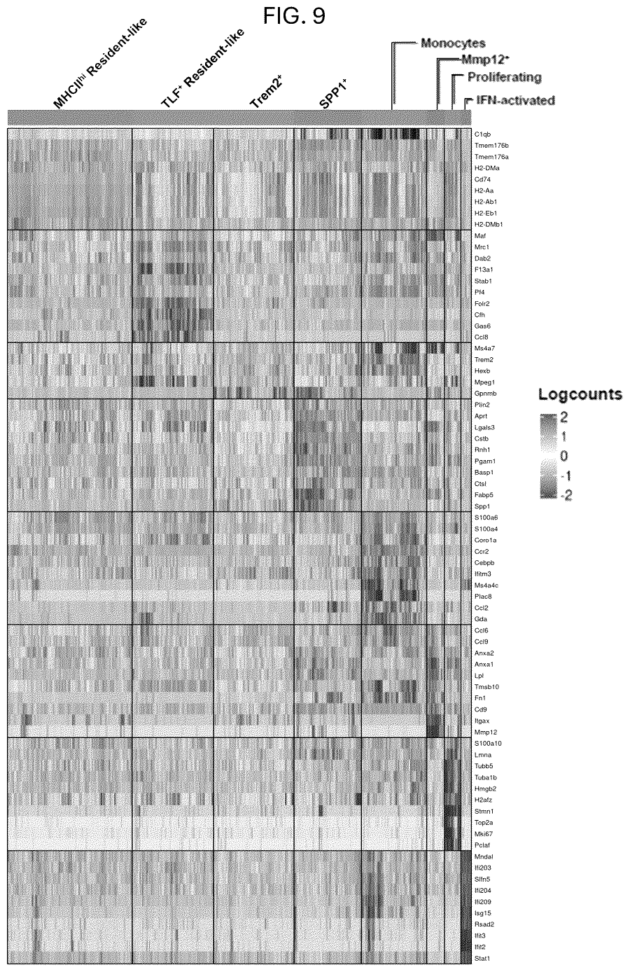

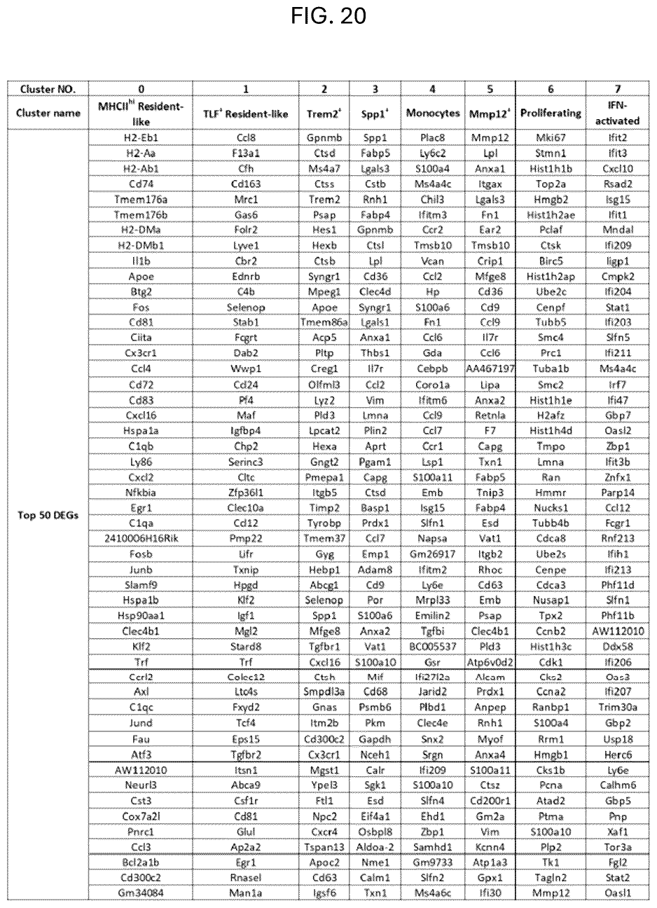

Monocytes and macrophages from all four samples were then sorted, pooled together, and re-clustered using UMAP analysis for functional subtypes of monocytes and macrophages. A total of eight sub-clusters were identified, including two featuring enriched genes resembling resident macrophages (resident-like), one with enriched Trem2 (Trem2⁺) expression, one with enriched Spp1 (Spp1⁺) expression, one with enriched genes of monocytes signatures (monocytes), one with enriched Mmp12 (Mmp12⁺) expression, one with enriched expression of interferon (IFN) response genes (IFN-activated), and another one with enriched expression of cell cycle genes (proliferating) (FIG. 3A, FIG. 9, and Table in FIG. 20).

FIGS. 3A-3C show macrophages in dystrophic mdx^{5cv} skeletal muscles contain multiple functional sub-clusters, among which the resident macrophage cluster expand in the absence of CCR2. Single-cell suspensions prepared from the quadriceps and diaphragm of mdx^{5cv} and mdx^{5cv}/Ccr2^{-/-} mice at 14 weeks of age were subjected to single-cell based RNA sequencing (scRNAseq) analysis. FIG. 3A shows Uniform Manifold Approximation and Projection (UMAP) dimension reduction analysis identifying sub-clusters of monocytes/macrophages in the quadriceps and diaphragm of mdx^{5cv} and mdx^{5cv}/Ccr2^{-/-} mice. FIG. 3B shows violin plots showing featured gene expression by different monocyte/macrophage sub-clusters in the quadriceps and diaphragm of mdx^{5cv} and mdx^{5cv}/Ccr2^{-/-} mice. FIG. 3C shows bar graphs showing the fraction of each sub-cluster to total monocytes/macrophages.

FIG. 9 shows heatmap depicting top 10 differentially expressed genes in individual monocyte and macrophage clusters in mdx^{5cv} and mdx^{5cv}/Ccr2^{-/-} quadriceps and diaphragm muscles.

FIG. 20 shows a table describing top 50 differentially expressed genes of monocytes and macrophage sub-clusters in mdx^{5cv} and mdx^{5cv}/Ccr2^{-/-} quadriceps and diaphragm muscles.

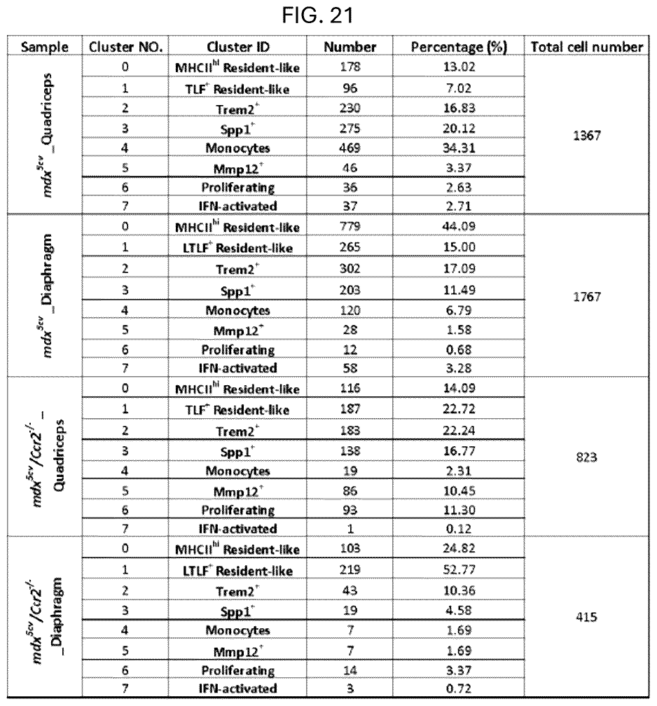

Previous studies across steady-state tissues, including skeletal muscle, have identified three clusters of resident macrophages which are defined by distinct expression of Timd4, Lyve1, Folr2, Ccr2^{-/-}, and MHCII genes. They are Timd4⁺ and/or Lyve1⁺ and/or Folr2⁺ (TLF⁺) Ccr2^{□}MHCII^{lo}, TLF^{□}Ccr2^{-/-}MHCII^{hi}, and TLF^{□}Ccr2^{□}MHCII^{hi} (24, 29, 30). The TLF expression marks primarily the embryo-derived resident macrophages (24, 29, 30) which can persist into adulthood through proliferative self-renewal. Similarly, our scRNAseq analysis identified two resident-like macrophage sub-clusters: one featuring enriched expression of TLF (TLF⁺) while the other featuring a high-level expression of MHCII genes (MHCII^{hi}) (FIG. 3B and Table S4). The TLF⁺ resident-like macrophages also expressed the highest level of Cdl63, Mrc1 and Adgre1 while a very low level of Cer2 (FIG. 3B). On the other hand, the MHCII^{hi} resident-like macrophages expressed a higher level of Ccr2 (FIG. 3B). Interestingly, the fraction of TLF⁺ resident macrophages increased dramatically in mdx^{5cv}/Ccr2^{-/-} muscles as compared to mdx^{5cv} muscles (22.72% vs. 7.02% in quadriceps and 52.77% vs. 15.00% in diaphragm) (FIG. 3C and Table in FIG. 21), demonstrating a predominant expansion of this embryo-derived resident macrophages in the absence of CCR2-mediated inflammatory monocyte/macrophage infiltration. FIG. 21 shows a table describing number and percentage of individual monocytes and macrophage sub-clusters identified by scRNAseq. The fraction of proliferating macrophages was also increased in mdx^{5cv}/Ccr2^{-/-} muscles as compared to mdx^{5cv} muscles (11.30% vs. 2.63% in quadriceps and 3.37% vs. 0.68% in diaphragm) (FIG. 3C and Table FIG. 21), suggesting that the expansion of resident macrophages may be via increased proliferation. Both monocytes and IFN-activated macrophages were diminished in mdx^{5cv}/Ccr2^{-/-} muscles compared to mdx^{5cv} muscles (Monocytes: 2.31% vs. 34.31% in quadriceps and 1.69% vs. 6.79% in diaphragm; IFN-activated macrophages: 0.12% vs. 2.71% in quadriceps and 0.72% vs. 3.28% in diaphragm.) (FIG. 3C and Table in FIG. 21), suggesting that the IFN-activated macrophages are differentiated from infiltrating monocytes.
C. Resident macrophages undergo pathogenic activation in mdx^{5cv}/Ccr2^{-/-} quadriceps and diaphragm.

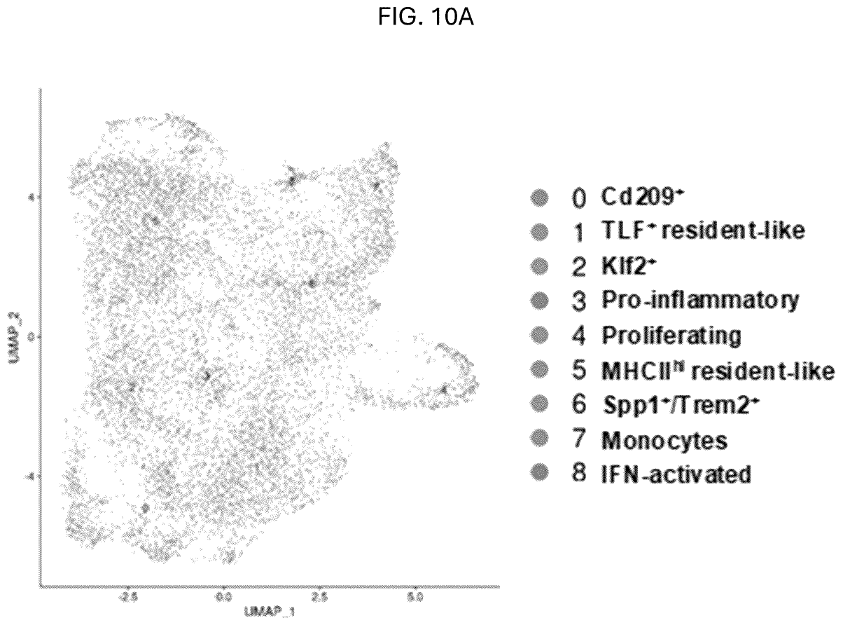

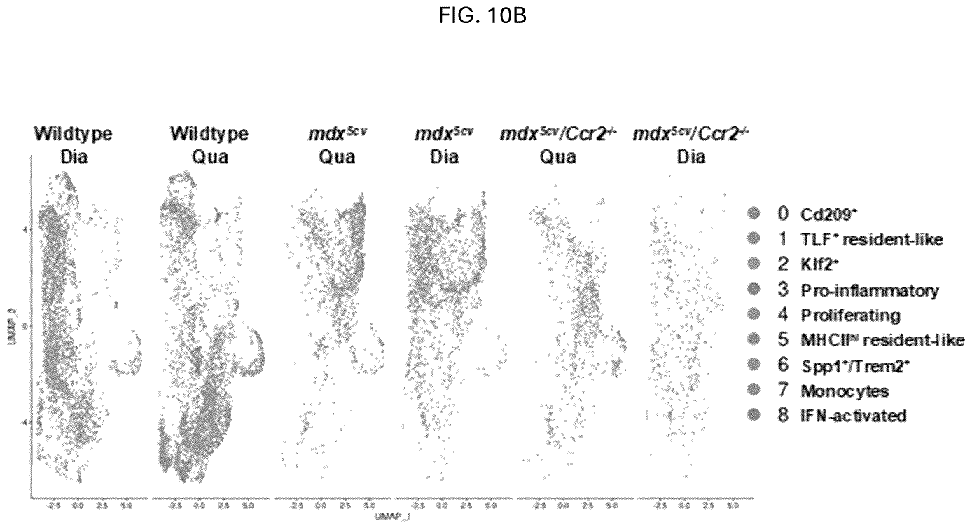

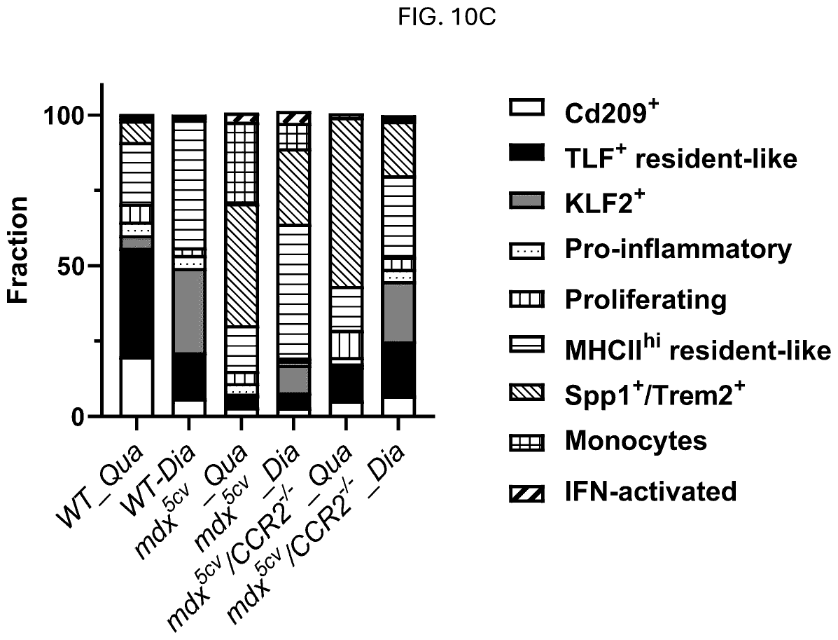

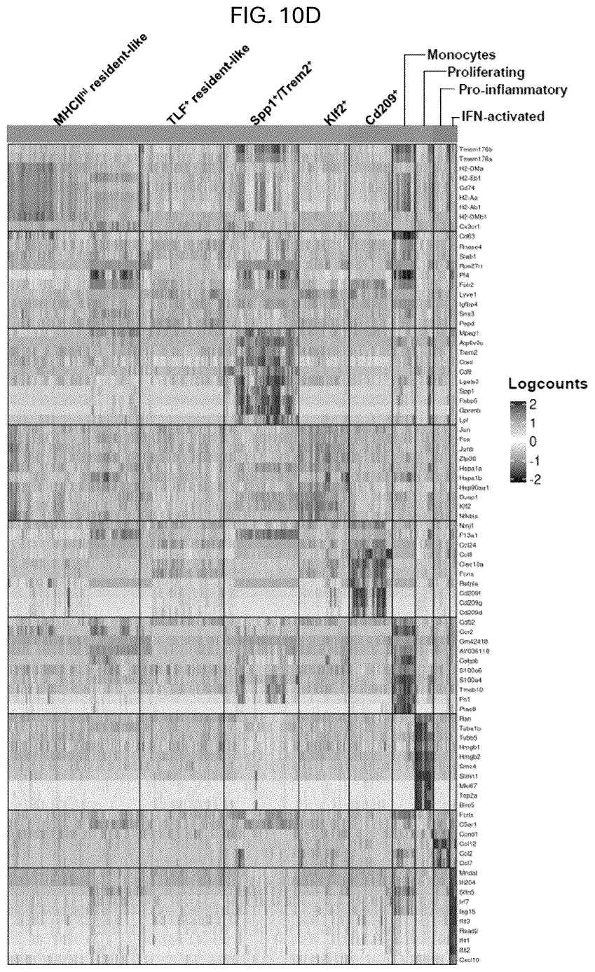

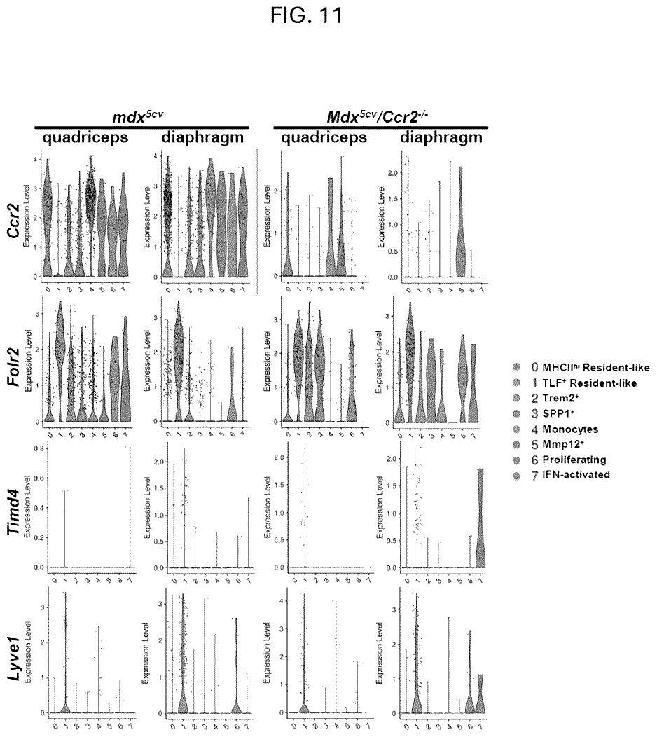

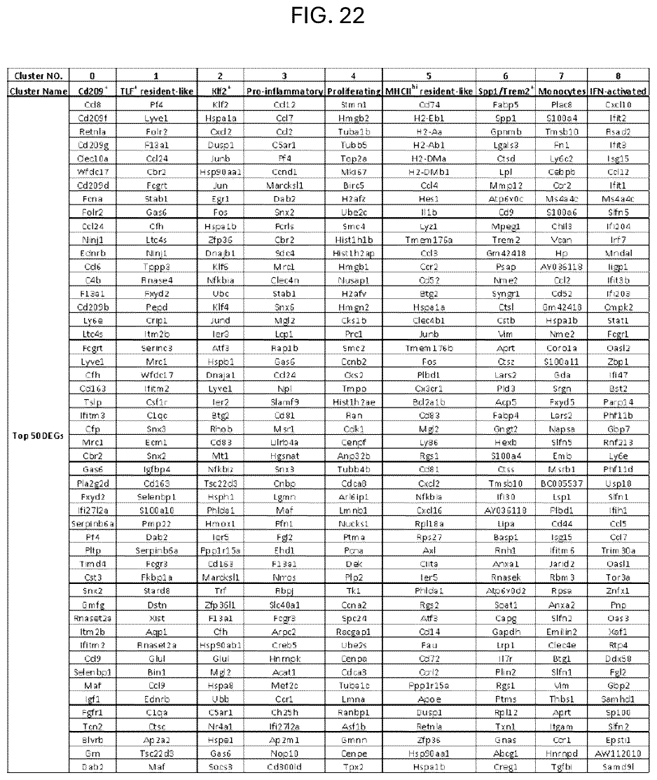

Recent studies have reported that TREM2⁺SPP1⁺ macrophages with high expression of Gpnmb, Fabp5 and Cd63 are enriched in the fibrotic tissues, where they play a pro-fibrotic role (31-33). Trem2⁺ and Spp1⁺ macrophage clusters, with enriched expression of Gpnmb, Fabp5 and Cd63, were also identified in the dystrophic muscles of both mdx^{5cv} and mdx^{5cv}/Ccr2^{-/-} mice (FIG, 3A, FIG. 9, and Table in FIG. 20). To address whether these subsets of macrophages are associated with dystrophic phenotype, we compared the monocytes and macrophages scRNAseq data generated by the current study with those by the previous study of wild-type (WT) mouse quadriceps and diaphragm (24). The two sets of scRNAseq data were first integrated to correct batch differences and then subjected to UMAP analysis to compare the transcriptome differences of monocytes and macrophages between healthy and dystrophic muscles. A total of 9 sub-clusters were identified and named by their differentially expressed genes (DEGs) (FIG. 10, and Table in FIG. 22). FIGS. 10A and 10B show UMAP analysis of scRNAseq data showing macrophage subclusters in wild-type, mdx^{5cv} and mdx^{5cv}/Ccr2^{-/-} quadriceps (Qua) and diaphragm (Dia) muscles. FIG. 10C shows bar graphs showing the percentage of each macrophage subcluster quantified from scRNAseq data. FIG. 10D shows heatmap depicting top 10 differentially expressed genes in individual monocyte/macrophage sub-clusters wild-type, mdx^{5cv} and mdx^{5cv}/Ccr2^{-/-} quadriceps and diaphragm muscles. FIG. 22 shows a table describing top 50 differentially expressed genes of monocytes and macrophage sub-clusters in wildtype, mdx^{5cv} and mdx^{5cv}/Ccr2^{-/-} quadriceps and diaphragm muscles.

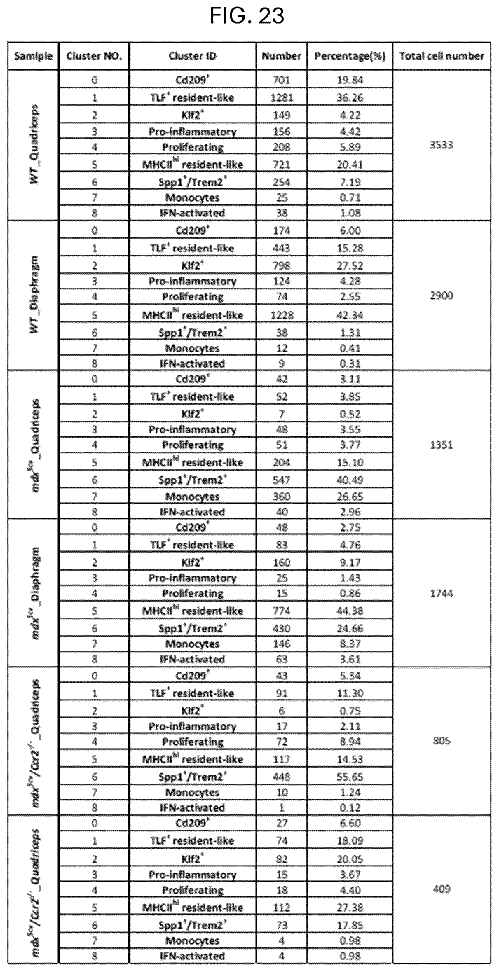

As expected, the Spp1⁺/Trem2⁺ cluster showed enriched expression of Cd9, Spp1,Gpnmb, Fabp5, and Cd63, supporting its pro-fibrotic function. Importantly, while the fraction of the Spp1⁺/Trem2⁺ cluster was very small in WT muscles (7.19% in quadriceps and 1.31% in diaphragm), it increased markedly in both mdx^{5cv} (40.49 % in quadriceps and 24.66% in diaphragm) and mdx^{5cv}/Ccr2^{-/-} muscles (55.65% in quadriceps and 17.85% in diaphragm) (FIG. 10C and Table in FIG. 23). FIG. 23 shows a table describing number and percentage of monocytes and individual sub-cluster of macrophages identified by scRNAseq in wild-type (WT), mdx^{5cv} and mdx^{5cv}/Ccr2^{-/-} quadriceps and diaphragm muscles. Therefore, the Spp1⁺/Trem2⁺ macrophages appear to be associated with the injured or dystrophic nature of skeletal muscle, and the macrophages in both mdx^{5cv} and mdx^{5cv}/Ccr2^{-/-} muscles undergo pathogenic activation. To assess the potential difference in the origin of Spp1⁺/Trem2⁺ macrophages between mdx^{5cv} and mdx^{5cv}/Ccr2^{-/-} muscles, we compared the expression of TLF genes and Ccr2 by different macrophage clusters using violin plots, as the TLF expression primarily marks embryo-derived resident macrophages (24, 29, 30), while the Ccr2 expression marks macrophages of HSC origin as CCR2 is required to the recruitment of muscle infiltrating macrophages (19). The Ccr2 gene targeting does not affect the first 39 nucleotides of the coding region and 5′ untranslated region of Ccr2 mRNA (34), so its expression is still detectable by scRNAseq in cells from Ccr2 deficient mice. Significantly, Spp1⁺/Trem2⁺ macrophages expressed Ccr2 in mdx^{5cv} but not mdx^{5cv}/Ccr2^{-/-} muscles, while monocytes expressed Ccr2^{-/-} in both mdx^{5cv} and mdx^{5cv}/Ccr2^{-/-} muscles (FIG. 11). FIG. 11 shows violin plots showing the expression of Ccr2, Folr2, Timd4, and Lyve1 by different macrophage clusters in the quadriceps and diaphragm of mdx^{5cv} and mdx^{5cv}/Ccr2^{-/-} mice at 14 weeks of age.

Instead, the Spp1⁺/Trem2⁺ macrophages in mdx^{5cv}/Ccr2^{-/-} muscles expressed a significantly higher level of Folr2 than in mdx^{5cv} muscles (Fig S4). Therefore, the HSC origin contributes to the Spp1⁺/Trem2⁺ macrophages in mdx^{5cv} muscles but not in mdx^{5cv}/Ccr2^{-/-} muscles.

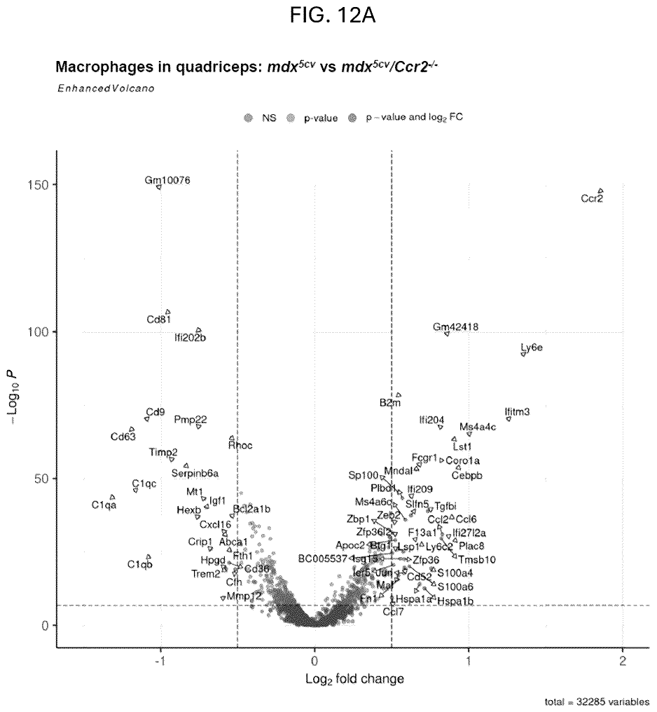

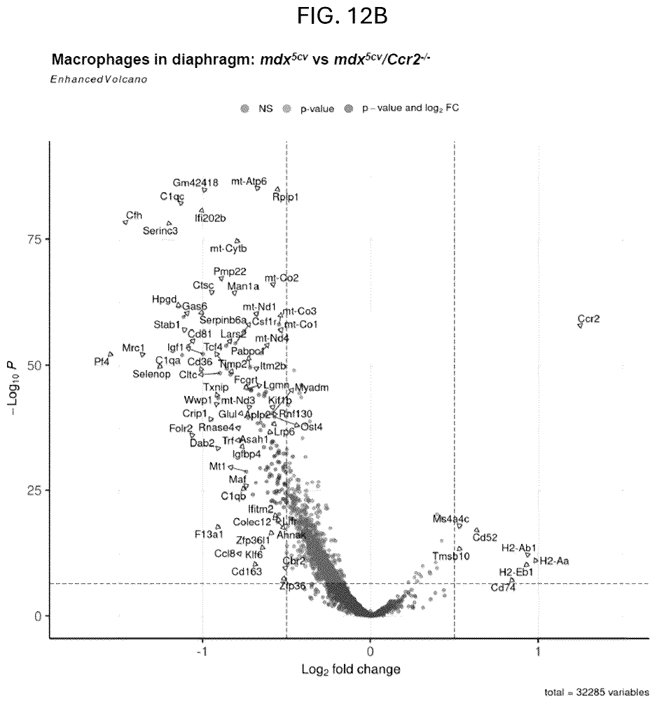

Macrophages can influence inflammation, necrosis, regeneration, and fibrosis in dystrophic muscles by producing pro-inflammatory, anti-inflammatory, pro-regenerative, and pro-fibrotic cytokines and growth factors, such as iNOS, TNF-α, IL-1α, IL-1β, IL-6, IL-10, IGF-1, TFG-β, and osteopontin (15, 18, 19, 27, 35-40). The infiltrating macrophages appear more pro-inflammatory and generally pathogenic in mdx muscles, as blocking macrophages infiltration reduces muscle fibrosis, increases muscle regeneration, and improves muscle function at early ages (8, 9). To further address whether resident macrophages undergo pathogenic activation in mdx^{5cv}/Ccr2^{-/-} muscles, macrophage transcriptome data from mdx^{5cv} quadriceps (1,367 cells) and diaphragm (1,767 cells) were compared with the data from mdx^{5cv}/Ccr2^{-/-} quadriceps (823 cells) and diaphragm (415 cells), respectively. A total of 32,285 genes were detected and compared for differentially expressed genes (DEGs, log₂FC (Fold Change)≥0.5, p<0.05). Among these genes, the number of DEGs identified was 44 in mdx^{5cv} quadriceps vs 25 in mdx^{5cv}/Ccr2^{-/-} quadriceps, and 8 in mdx^{5cv} diaphragm vs 135 in mdx^{5cv}/Ccr2^{-/-} diaphragm (FIG. 12). FIG. 12 shows volcano plots comparing the transcriptome of macrophages between mdx^{5cv} and mdx^{5cv}/Ccr2^{-/-} quadriceps (FIG. 12A) and diaphragm (FIG. 12B). Differentially expressed genes (DEGs, p<0.05, Log2FC≥0.5) are indicated.

The DEGs of macrophages in mdx^{5cv} muscles largely resembled the featured genes of monocytes, while the DEGs of macrophages in mdx^{5cv}/Ccr2^{-/-} muscles largely resembled the featured genes of resident-like macrophages (FIG. 12 and Table in FIG. 20). Therefore, the DEGs mainly reflect the differences in the fractions of monocyte and resident-like macrophage clusters between mdx^{5cv} and mdx^{5cv}/Ccr2^{-/-} muscles (FIG. 3C). We next focused the comparison on the expression of the genes that regulate inflammation and fibrosis, including pro-inflammatory genes (Nos2, Tnf, Illa, Illb, Il6, Ccl2, Ccl7, and Cxcl2), anti-inflammatory genes (Arg1, Il10, Il4, Tgfb1, and Igfl), pro-fibrotic genes (Pdgfa, Fn1, Ccn2, Spp1, Pparg, Tgfb1 and Igfl), and fibrosis regulatory genes (Tgfb1, Ccn2, Pdgfa, Mmp2,Mmp9,Mmp12,Mmp14, Timp1, and Timp2).

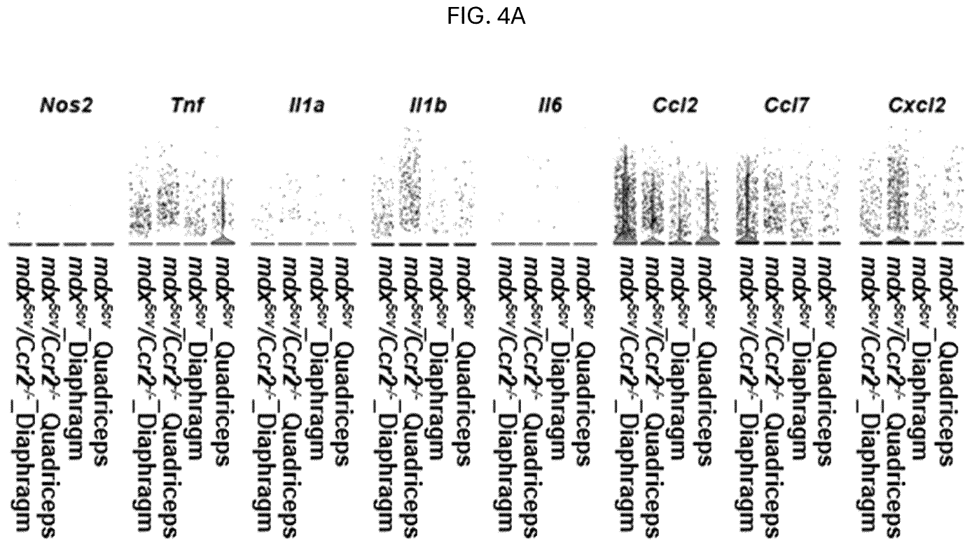

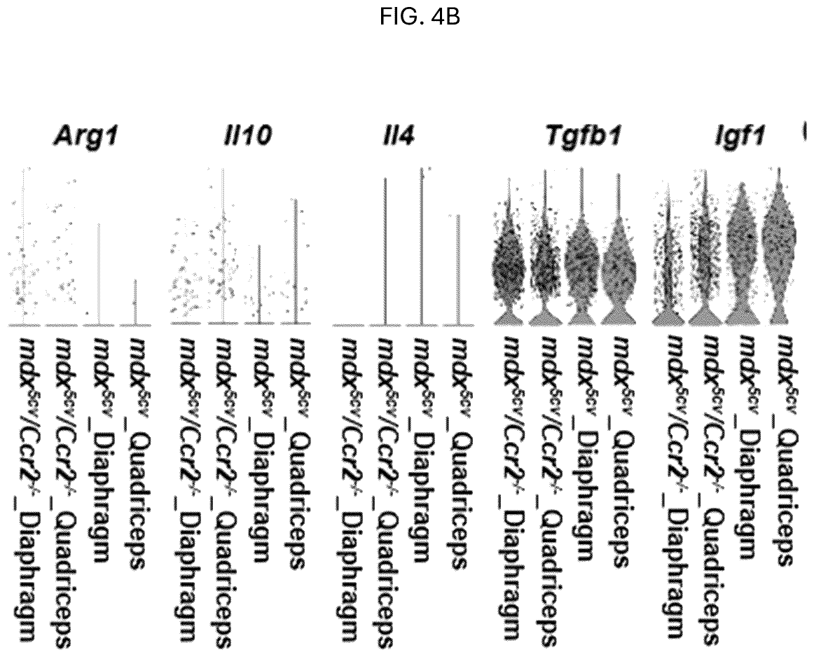

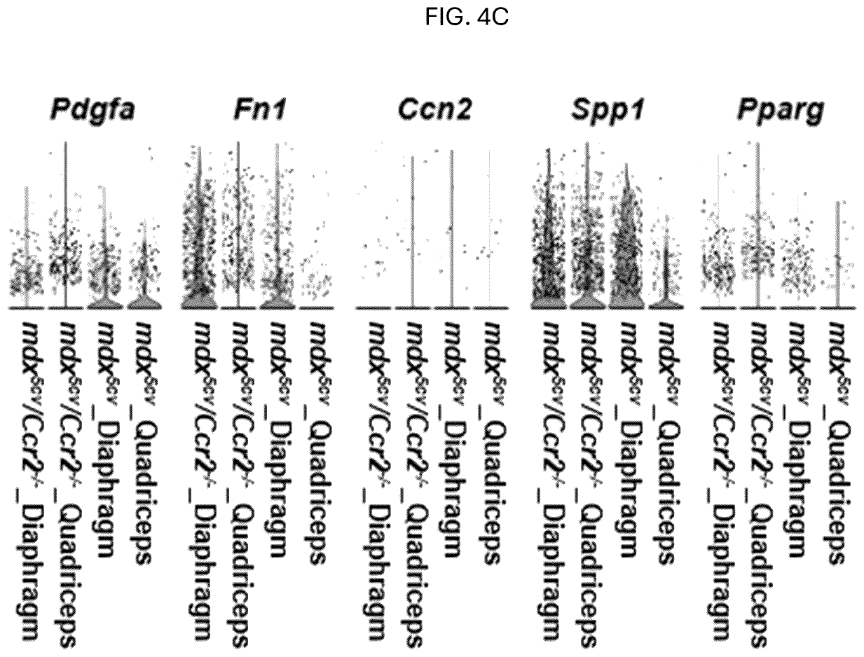

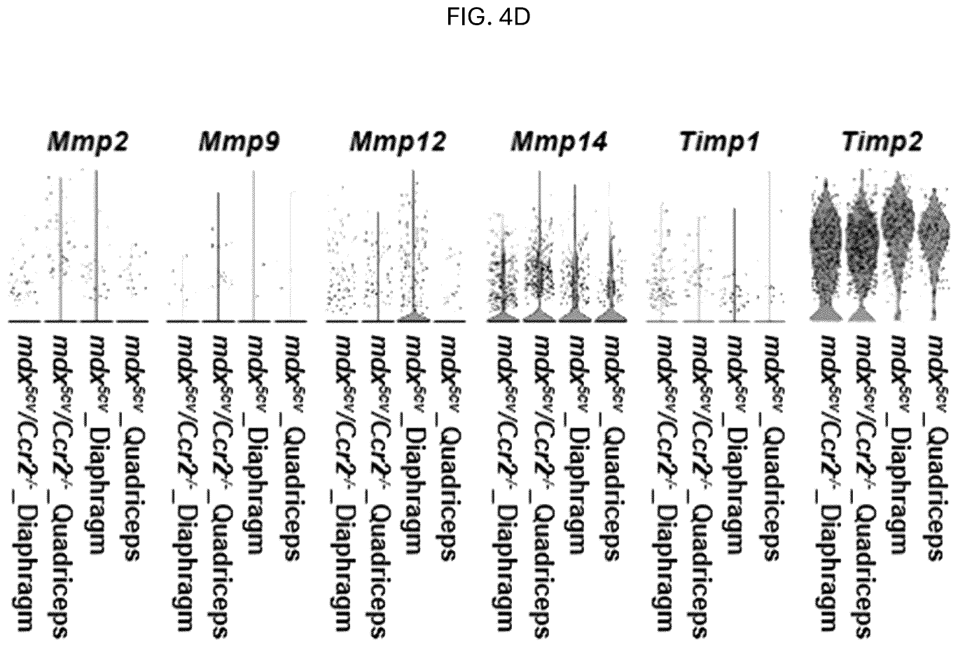

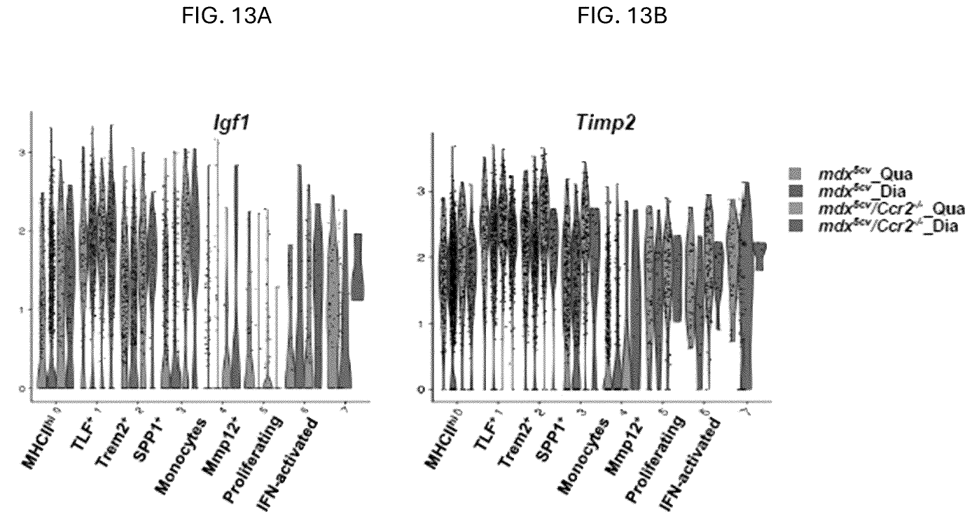

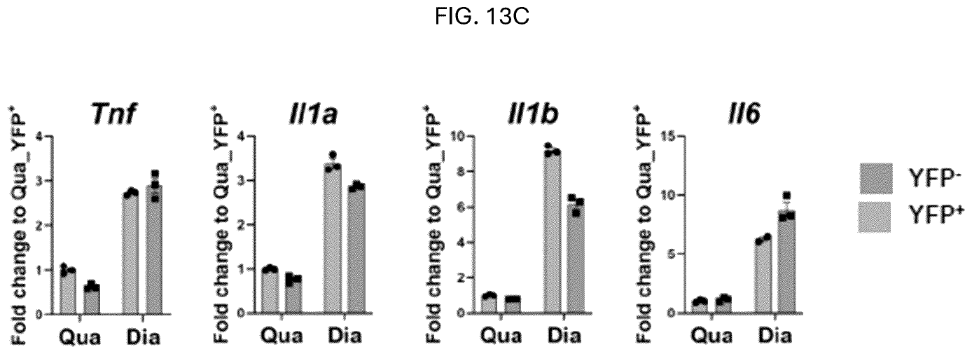

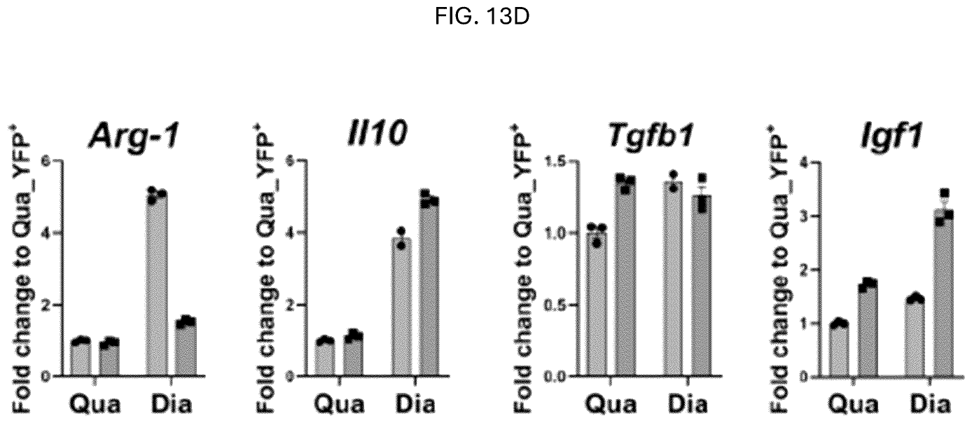

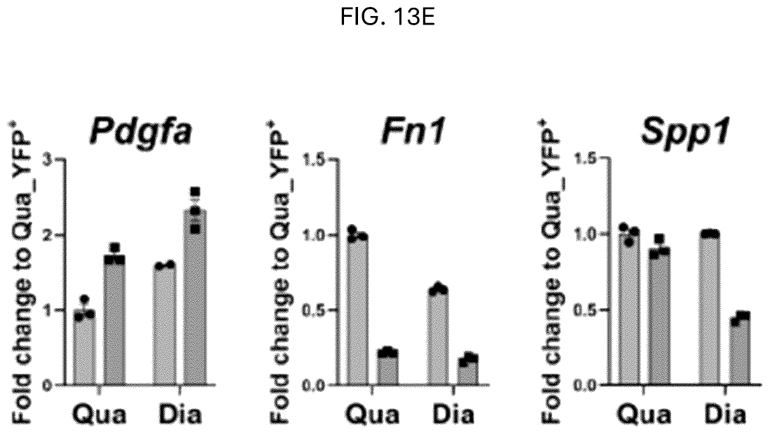

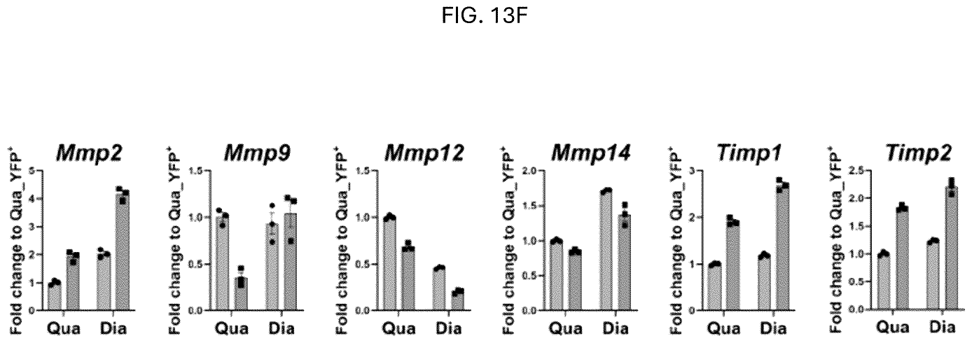

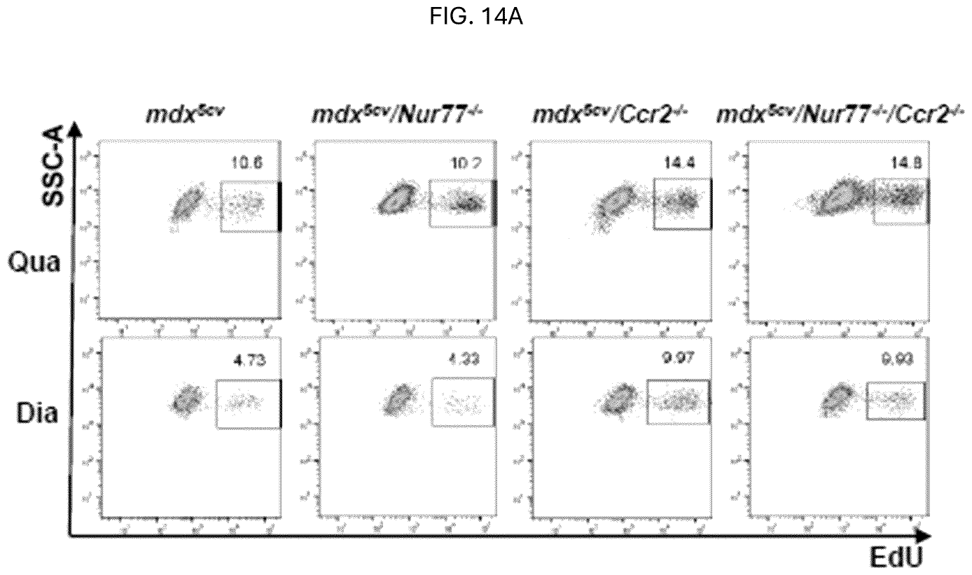

Violin plots showed that most of these genes were expressed at a similar average level by macrophages in the four muscle samples, except for Igf1 and Timp2 (FIG. 4). FIGS. 4A-4D show intramuscular macrophages express a similar level of the genes regulating inflammation and fibrosis between mdx^{5cv} and mdx^{5cv}/Ccr2^{-/-} quadriceps or diaphragm. Violin plots of scRNAseq data showing the expression of pro-inflammatory genes (FIG. 4A), anti-inflammatory genes (FIG. 4B), pro-fibrotic genes (FIG. 4C), and fibrosis regulatory genes (FIG. 4D) by macrophages of different muscle samples as indicated. Therefore, although the origins of the Ly6C^{lo} macrophages between mdx^{5cv} and mdx^{5cv}/Ccr2^{-/-} muscles are different, with the former predominantly from infiltrated macrophages while the later mainly from resident macrophages, they appear to have similar functional properties with respect to inflammation and fibrosis. Interestingly, the expression of Igf1 and Timp2 by total macrophages was higher in mdx^{5cv}/Ccr2^{-/-} quadriceps and diaphragm than in mdx^{5cv} controls. This was most likely contributed by the higher TLF⁺ resident macrophage fraction in mdx^{5cv}/Ccr2^{-/-} muscles (FIG. 3A and FIG. 3C), as TLF⁺ resident macrophages expressed the highest levels of Igf1 and Timp2 (FIGS. 13A-B). FIG. 13A and FIG. 13B show violin plot of scRNAseq data showing the expression of Igf1 (FIG. 13A) and Timp2 (FIG. 13B) gene by different macrophage subclusters. FIG. 13C to F show quantitative reverse transcription-PCR (qRT-PCR) analysis of the mRNA expression of pro-inflammatory (FIG. 13C), anti-inflammatory (FIG. 13D), pro-fibrotic (FIG. 13E) and fibrosis regulatory (FIG. 13F) genes by YFP⁺ and YFP⁻ Ly6C^{lo} macrophages in Flt3^{Cre}/Rosa26^{LSL-YFP}/mdx^{5cv}/Ccr2^{-/-} quadriceps (Qua) and diaphragm (Dia). YFP⁺ and YFP⁻ Ly6C^{lo} macrophages each were sorted and pooled from 10 Flt3^{Cre}/Rosa26^{LSL-YFP}/mdx^{5cv}/Ccr2^{-/-} mice. PCR analysis was performed at triplicates. To further confirm that Ly6C^{lo} intramuscular macrophages of different origins play similar pathogenic roles, YFP⁺ (HSC origin) and YFP⁻ (non-HSC embryonic origin) Ly6C^{lo} macrophages were sorted from Flt3^{cre}/Rosa26^{LSL-YFP}/mdx^{5cv}/Ccr2^{-/-} quadriceps and diaphragm muscles and subjected to qRT-PCR analysis of the genes regulating inflammation and fibrosis. As shown in FIGS. 13C-13F, most of these genes were expressed at comparable levels between YFP⁺ and YFP Ly6C^{lo} macrophages. Therefore, the resident macrophages in mdx^{5cv}/Ccr2^{-/-} quadriceps and diaphragm undergo pathogenic activation, similar to the macrophages in mdx^{5cv} controls.
D. The expansion of Ly6C^{lo} macrophages in mdx^{5cv}/Ccr2^{-/-} muscles is achieved by increased cell proliferation.

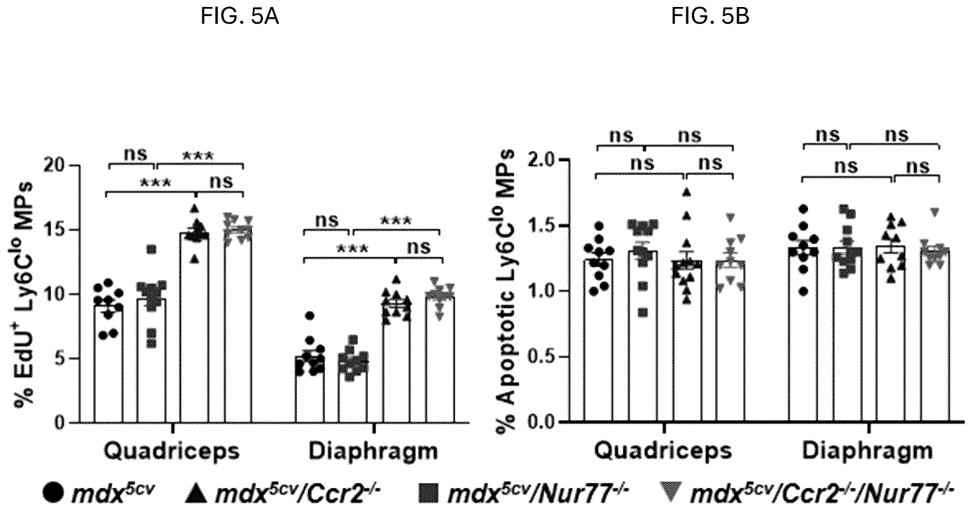

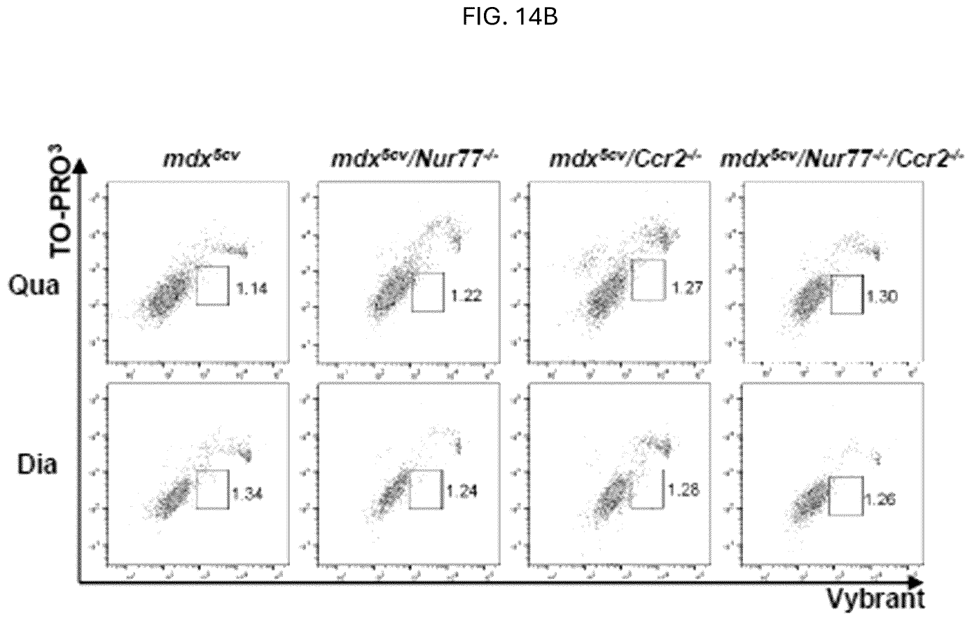

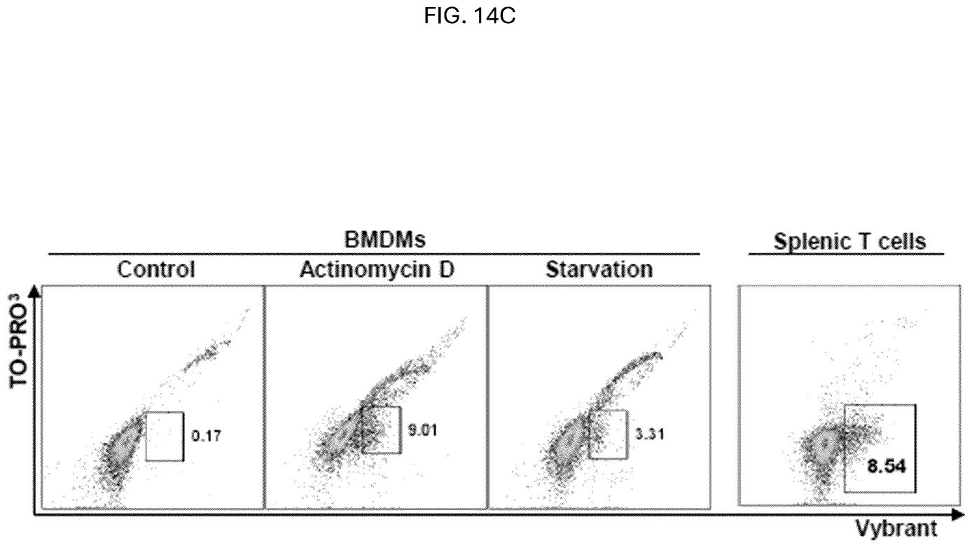

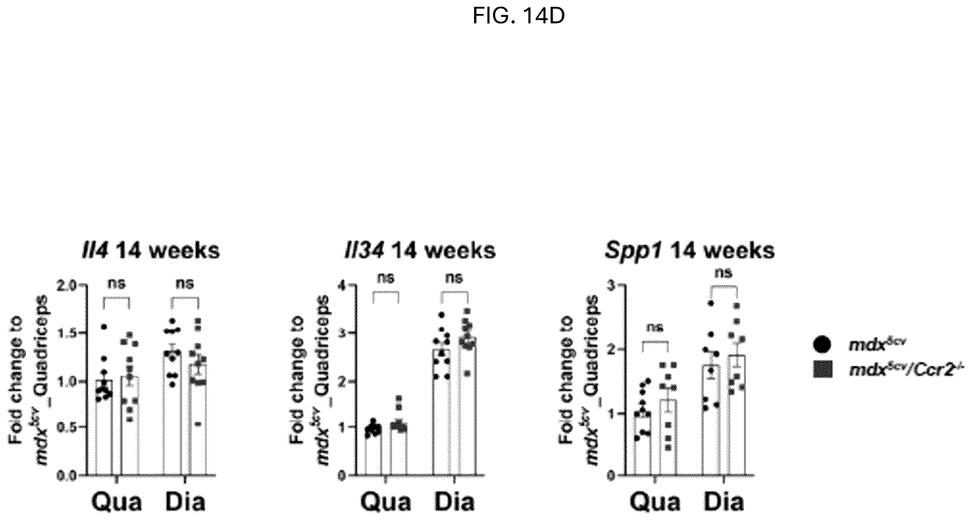

To address whether the expansion of Ly6C^{lo} macrophages in mdx^{5cv}/Ccr2^{-/-} muscles was resulted from increased proliferation and/or decreased apoptosis, we quantified the percentages of proliferating and apoptotic intramuscular macrophages, using 5-ethynyl 2′-deoxyuridine (EdU) incorporation assay and Vybrant Dye staining, respectively, and compared the results from mdx^{5cv}/Ccr2^{-/-} and mdx^{5cv}/Ccr2^{-/-}/Nur7^{-/-} mice with those from mdx^{5cv} and mdx^{5cv}/Nur77^{-/-} mice, respectively, at 14 weeks of age. The percentage of EdU⁺ Ly6C^{lo} macrophages increased significantly in mdx^{5cv}/Ccr2^{-/-} and mdx^{5cv}/Ccr2^{-/-}/Nur7^{-/-} quadriceps and diaphragm compared to mdx^{5cv} and mdx^{5cv}/Nur77^{-/-} controls, respectively (FIG. 5A and FIG. 14A), indicating that Ly6C^{lo} macrophage proliferation was increased in the absence of CCR2. FIGS. 5A-5B show the expansion of intramuscular Ly6C^{lo} macrophages is resulted from increased proliferation in dystrophic skeletal muscles in the absence of CCR2. Single-cell suspensions prepared from the quadriceps and diaphragm of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age were subjected to FACS analysis of proliferation by EdU incorporation assay (FIG. 5A) and apoptosis by Vybrant Dye staining (FIG. 5B). FIG. 5A shows bar graphs comparing the percentages of EdU⁺ macrophages in the quadriceps and diaphragm muscles among mice with different genotypes. FIG. 5B shows bar graphs showing comparisons of the percentages of apoptotic macrophages in the quadriceps and diaphragm muscle among mice with different genotypes. N=10 mice/group. ***p<0.001; ns: no significance. FIG. 14A and FIG. 14B show single-cell suspensions prepared from the quadriceps and diaphragm of mdx^{5cv}, mdx^{5cv}/Nur77^{-/-}, mdx^{5cv}/Ccr2^{-/-}, and mdx^{5cv}/Ccr2^{-/-}/Nur77^{-/-} mice at 14 weeks of age were subjected to FACS analysis of proliferation by EdU incorporation assay (FIG. 14A) and apoptosis by Vybrant Dye staining (FIG. 14B). FIG. 14A shows represented dot plots showing EdU+ proliferating macrophages. FIG. 14B shows represented dot plots showing apoptotic macrophages. FIG. 14C shows represented dot plots showing apoptosis of BMDMs induced by actinomycin D treatment (0.5 mM, 12 hours) or by CSF-1 starvation (24 hours), and apoptosis of mouse splenic CD3⁺ T cells. FIG. 14D shows qRT-PCR analysis of the mRNA expression of Il4, Il34 and Spp1 by mdx^{5cv} and mdx^{5cv}/Ccr2^{-/-} quadriceps and diaphragm. n=10 per group. ns: no significance.

However, there was no significant difference in the percentage of apoptotic intramuscular Ly6C^{lo} macrophages between mdx^{5cv}/Ccr2^{-/-} and mdx^{5cv} mice or between mdx^{5cv}/Ccr2^{-/-}/Nur7^{-/-} and mdx^{5cv}/Nur77^{-/-} mice (FIG. 5B and FIG. 14B), indicating that the Ly6C^{lo} macrophage apoptosis was not affected by the absence of CCR2. Therefore, the expansion of Ly6C^{lo} macrophages in mdx^{5cv}/Ccr2^{-/-} muscles is achieved by increased cell proliferation.
E. The increased proliferation of intramuscular Ly6C^{lo} macrophages appears stimulated by CSF-1/CSF-1R signaling and CSF-1 is primarily produced by FAPs in mdx^{5cv}/Ccr2^{-/-} mice.

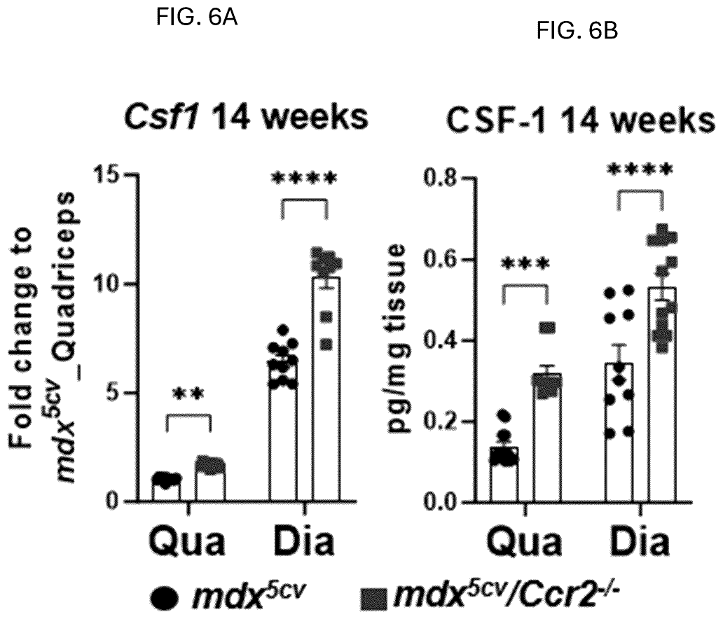

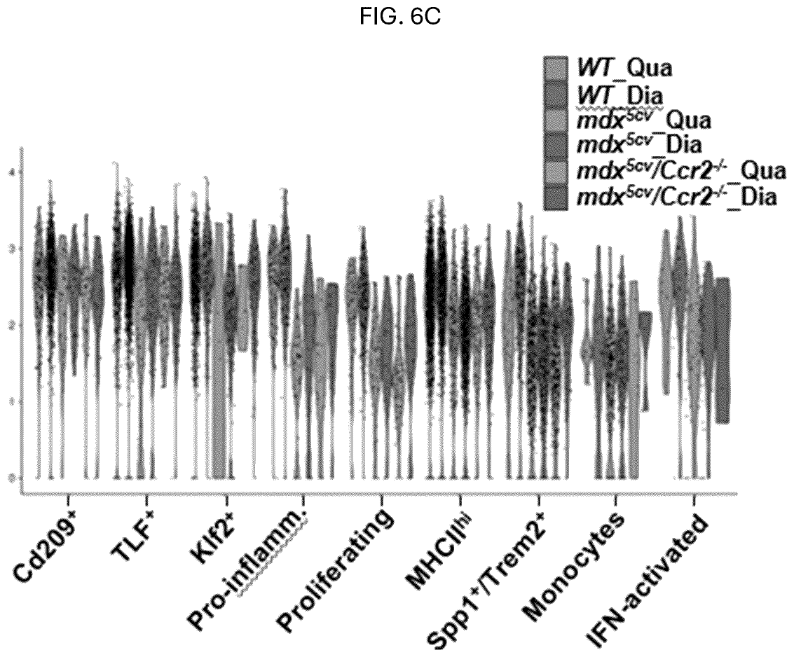

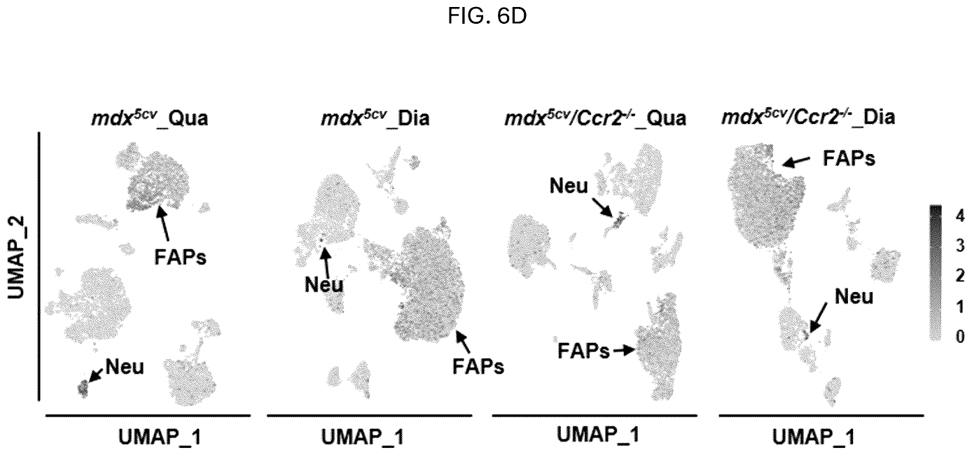

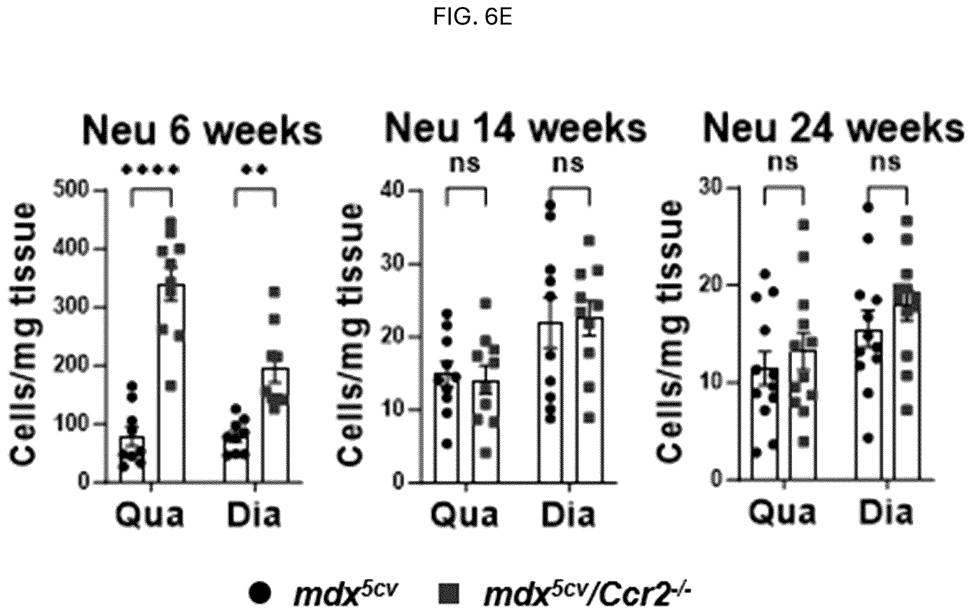

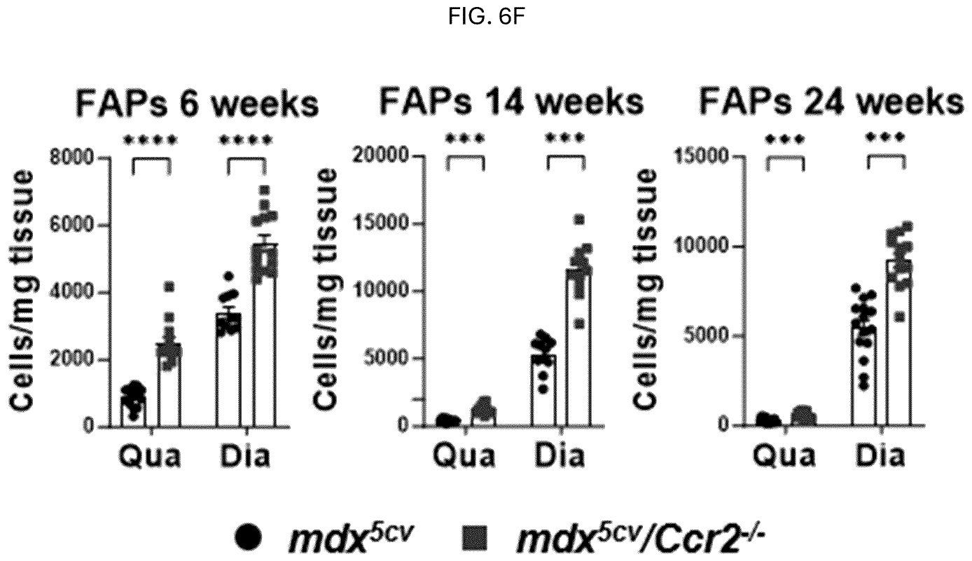

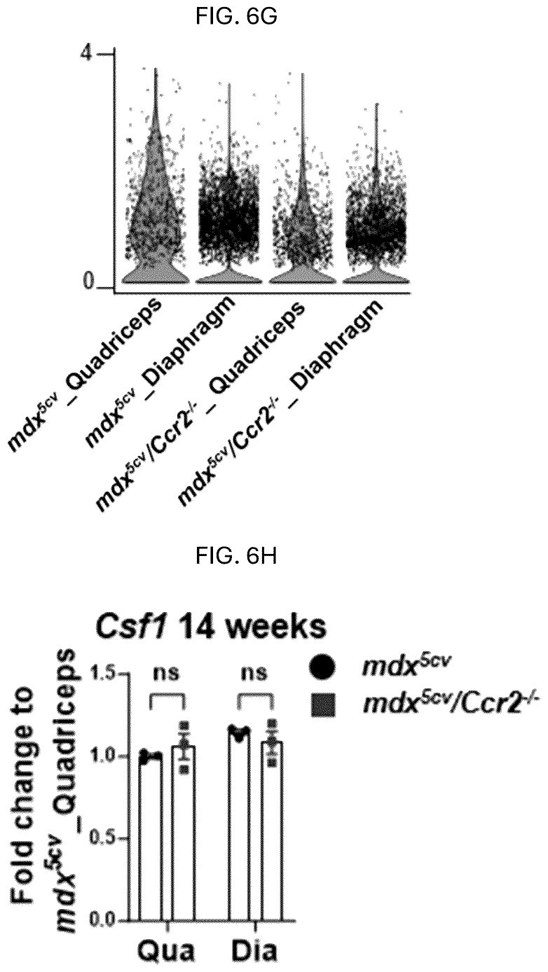

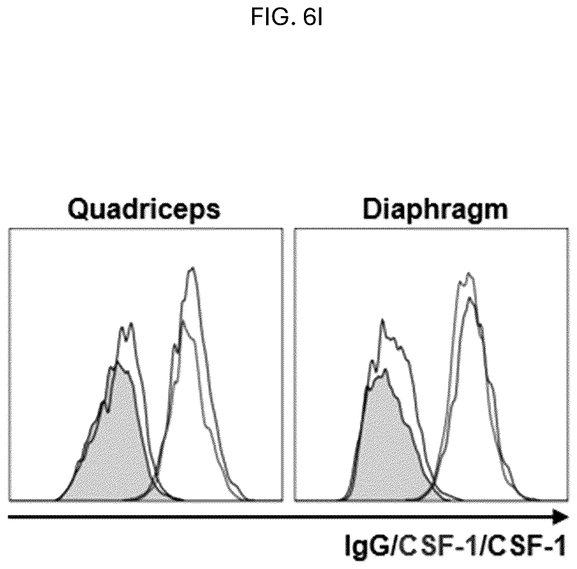

Macrophage proliferation can be stimulated by multiple signaling cytokines, including CSF-1, IL-4, IL-34, and Osteopontin (encoded by Spp1 gene) (40-42). Quantitative reverse transcription PCR (qRT-PCR) showed that whole-muscle expression of Csfl, but not the other genes, was significantly upregulated in mdx^{5cv}/Ccr2^{-/-} quadriceps and diaphragm, compared to mdx^{5cv} controls (FIG. 6A and FIG. 14C). FIGS. 6A-6I show a high intramuscular density of FAPs contributes to the increased CSF-1 production in mdx^{5cv}/Ccr2^{-/-} quadriceps and diaphragm. FIG. 6A and FIG. 6B show whole-muscle production of CSF-1 in the quadriceps and diaphragm between mdx^{5cv} and mdx^{5cv}/Ccr2^{-/-} mice at 14 weeks of age was determined by qRT-PCR for mRNA (FIG. 6A) and by ELISA for protein (FIG. 6B). N=10 mice/group. **p<0.01; ***p<0.001; ****p<0.0001. FIG. 6C shows violin plot of scRNAseq data showing the expression of Csflr by different monocyte macrophage sub-clusters. FIG. 6D shows feature plots of scRNAseq data showing that FAPs and neutrophils (arrows) are the major cellular sources of Csfl mRNA expression in the quadriceps (Qua) and diaphragm (Dia) of mdx^{5cv} and mdx^{5cv}/Ccr2^{-/-} mice. FIG. 6E and FIG. 6F show bar graphs showing comparisons of the cell densities, quantified by FACS analysis, of neutrophils (Neu) (FIG. 6E) and FAPs (FIG. 6F) in the quadriceps and diaphragm between mdx^{5cv} and mdx^{5cv}/Ccr2^{-/-} mice. N=10 mice/group. **p<0.01; ***p<0.001; ****p<0.0001; ns, no significance. FIG. 6G shows violin plot of scRNAseq data showing FAP expression of Csfl mRNA in the quadriceps and diaphragm of mdx^{5cv} and mdx^{5cv}/Ccr2^{-/-} mice. FIG. 6H shows qRT-PCR analysis of Csfl mRNA expression by FAPs sorted from the quadriceps and diaphragm of mdx^{5cv} and mdx^{5cv}/Ccr2⁻⁻ mice at 14 weeks of age. FAPs were sorted from 5 mice/group and combined for RNA preparation. FIG. 6I show histogram of FACS analysis showing the expression of CSF-1 by FAPs in the quadriceps and diaphragm of mdx^{5cv} and mdx^{5cv}/Ccr2^{-/-} mice at 14 weeks of age. Black line: IgG isotype control (mdx^{5cv}). Black line with grey filled: IgG isotype control (mdx^{5cv}/Ccr2^{-/-}). Red line: CSF-1 staining (mdx^{5cv}). Blue line: CSF-1 staining (mdx^{5cv}/Ccr2^{-/-}). Data presented represents 5 mice/group. Likewise, the CSF-1 protein expression also increased in mdx^{5cv}/Ccr2^{-/-} muscles compared to mdx^{5cv} controls (FIG. 6B). Moreover, Violin plot analysis of scRNAseq data showed that, among all the macrophage sub-clusters, the TLF⁺ resident-like sub-cluster expressed the highest level of the CSF-1 receptor (CSF-IR) gene, Csflr, and that the MHCII^{hi} resident-like sub-cluster also expressed a high level of Csflr (FIG. 6C). In addition, macrophages in uninjured WT muscles expressed a higher level of Csflr than those in chronically injured muscles (FIG. 6C). Therefore, the CSF-1/CSF-1R signaling may play an important role in stimulating the proliferation of resident macrophages in mdx^{5cv}/Ccr2^{-/-} muscles.

We next determined the cellular source of CSF-1 in the dystrophic muscles. Feature plot analysis of scRNAseq data showed that FAPs and neutrophils were the main cell types that express Csfl (FIG. 6D and FIG. 8B). FACS analysis of single-cell suspensions of quadriceps and diaphragm from mdx^{5cv}/Ccr2^{-/-} and mdx^{5cv} at ages 6, 14 and 24 weeks showed increased neutrophil density in mdx^{5cv}/Ccr2^{-/-} muscles only at 6 weeks (FIG. 6E), while increased FAP densities at all ages (FIG. 6F). Moreover, FAPs greatly outnumbered neutrophils in the dystrophic muscles, as determined both by fractions (FIG. 6D) and densities (FIGS. 6E-F). Therefore, FAPs are the primary cellular source of CSF-1 in the dystrophic muscles. Violin plots illustrated that the Csfl gene expression by FAPs at the single-cell level was not significantly different between mdx^{5cv}/Ccr2^{-/-} and mdx^{5cv} quadriceps or diaphragm. (FIG. 6G). Likewise, qRT-PCR also showed no significant difference (FIG. 6H). Furthermore, the FAP expression of CSF-1 protein, as assessed by FACS analysis with intracellular staining, was not significantly different either (FIG. 61). Therefore, the increased level of CSF-1 at whole muscle level is most likely contributed by the persistently higher density of FAPs in mdx^{5cv}/Ccr2^{-/-} quadriceps and diaphragm than in mdx^{5cv} controls (FIG. 6F).

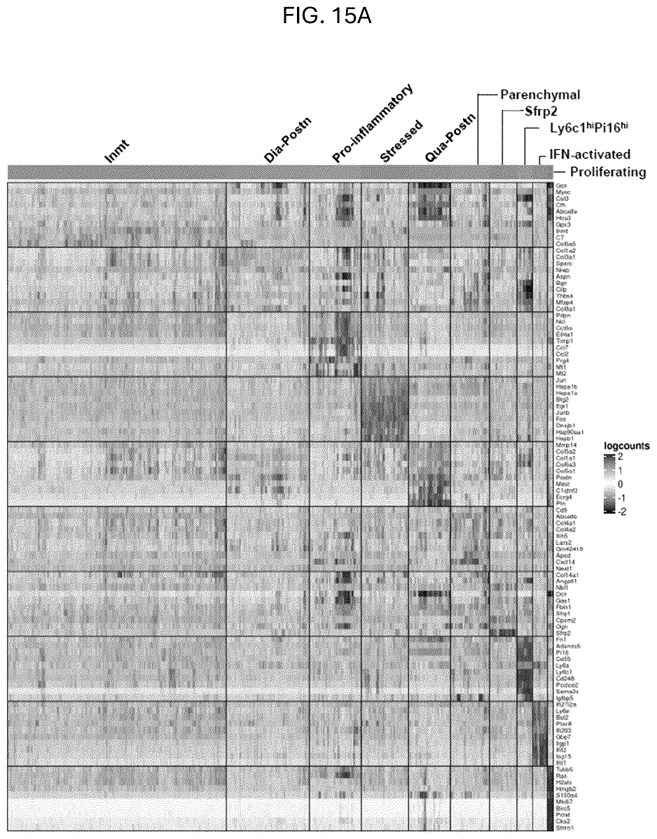

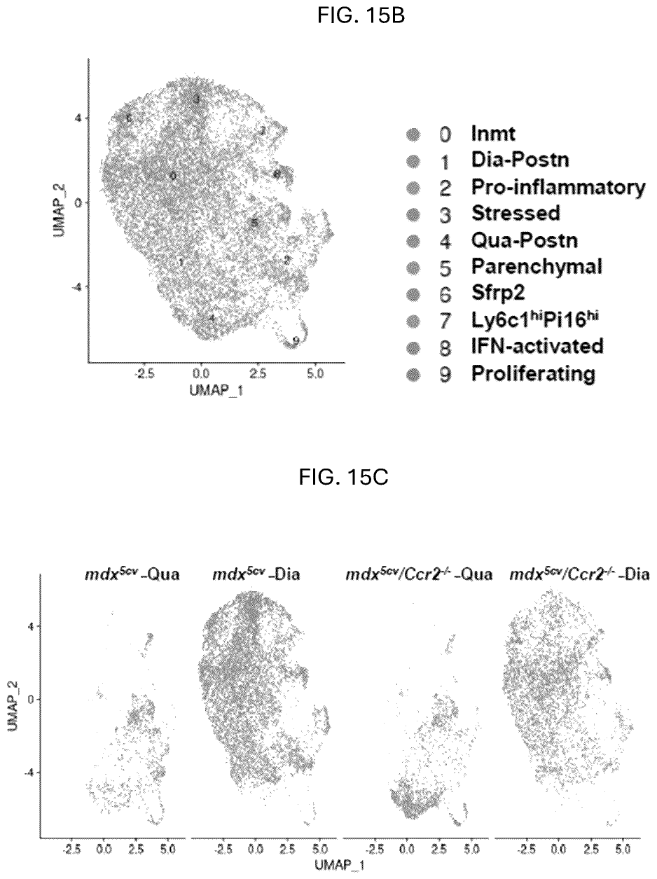

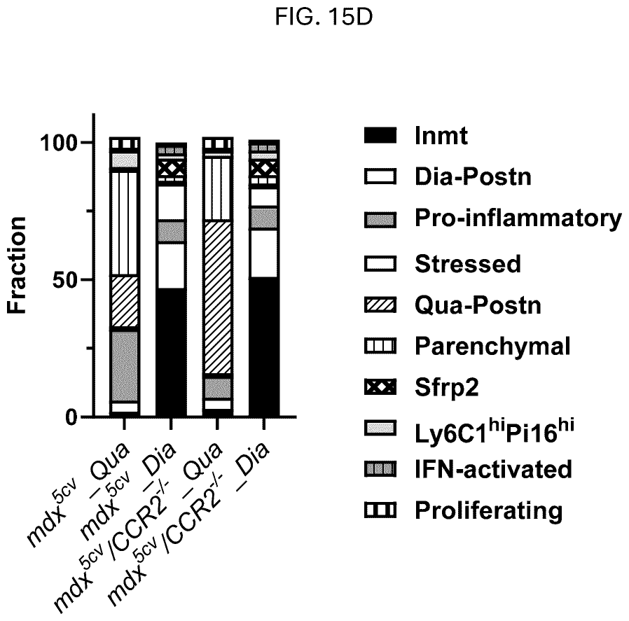

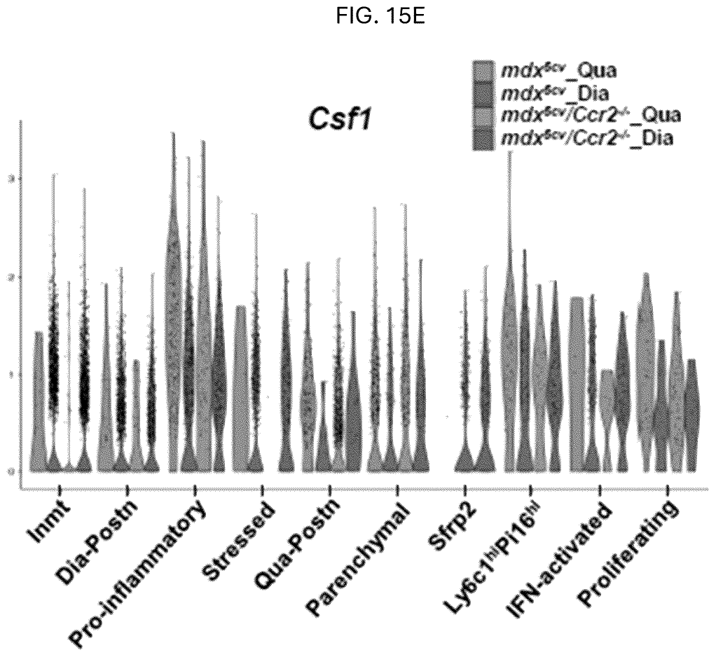

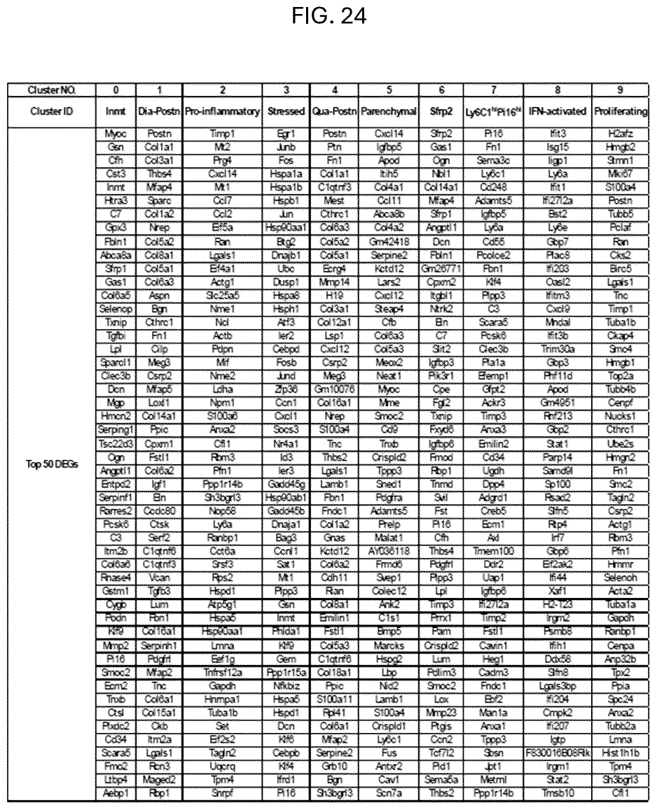

Recently, Babaeijandaghi et al. suggested a role for the dipeptidyl peptidase IV (DPPIV)⁺ FAP subtype as CSF-1-producing niche cells for self-renewing resident macrophages in steady-state skeletal muscle (43). We also identified by scRNAseq a similar FAP cluster, named Ly6c1^{hi}Pi16^{hi} cluster, in both WT and mdx^{5cv} muscles, which expressed a high level of Ly6c1, Pi16, and Dpp4 (44), resembling adventitial fibroblasts identified across different tissues (45). We then addressed whether this FAP subtype is also the main cellular source of CSF-1. To this end, FAPs identified by scRNAseq analysis from both mdx^{5cv} and mdx^{5cv}/Ccr2^{-/-} quadriceps and diaphragm were pooled and re-clustered using UMAP analysis for functional subtypes. A total of ten sub-clusters were identified (FIG. 15A-D, and Table in FIG. 24). FIG. 15A shows heatmap depicting top 10 differentially expressed genes in individual FAP subclusters in mdx^{5cv} and mdx^{5cv}/Ccr2^{-/-} quadriceps and diaphragm muscles. FIGS. 15B and 15C UMAP analysis of scRNAseq data showing FAP subclusters in mdx^{5cv} and mdx^{5cv}/Ccr2^{-/-} quadriceps (Qua) and diaphragm (Dia) muscles. FIG. 15D shows bar graphs showing the percentage of each FAP sub-cluster quantified from scRNAseq data. FIG. 15E shows violin plot showing the expression of Csfl gene by each sub-clusters in mdx^{5cv} and mdx^{5cv}/Ccr2^{-/-} quadriceps (Qua) and diaphragm (Dia) muscles. FIG. 24 shows a table describing top 50 differentially expressed genes of FAP sub-clusters in mdx^{5cv} and mdx^{5cv}/Ccr2^{-/-} quadriceps and diaphragm muscles.

Four clusters were shared by FAPs from all samples, including Ly6c1^{hi}Pi16^{hi}Dpp4⁺ cluster, parenchymal cluster featuring Cxcl14 and Col4al, pro-inflammatory cluster featuring Ccl2 and Timp1, and proliferating cluster featuring cell-cycle genes (FIGS. 15C-D, and Table in FIG. 24). FAP cluster specificity was observed between quadriceps and diaphragm, but not between mdx^{5cv} and mdx^{5cv}/Ccr2^{-/-} genotypes. Five clusters were diaphragm specific: a cluster featuring Inmt (Inmt), a cluster featuring Postn (Dia-Postn), a cluster featuring genes responding to stress (Stressed), a cluster featuring Sfrp2 (Sfrp2), and a cluster featuring IFN-activated genes (IFN-activated). A distinct Postn-enriched cluster was also identified in quadriceps (Qua-Postn) (FIGS. 15C-D, and Table in FIG. 24). We next determined the expression of Csfl by each individual FAP cluster with violin plot. Csfl was expressed by all FAP clusters, with the highest expression by pro-inflammatory and Ly6c1^{hi}Pi16^{hi}Dpp4⁺ clusters (FIG. 15E). However, the Ly6c1^{hi}Pi16^{hi}Dpp4⁺ cluster was very small (FIG. 15D) and thus unlikely to be the primary cellular source of Csfl in dystrophic muscles of mdx^{5cv} and mdx^{5cv}/Ccr2^{-/-} mice. Other FAP subtypes, in particular the pro-inflammatory cluster, may contribute significantly to the Csfl expression.

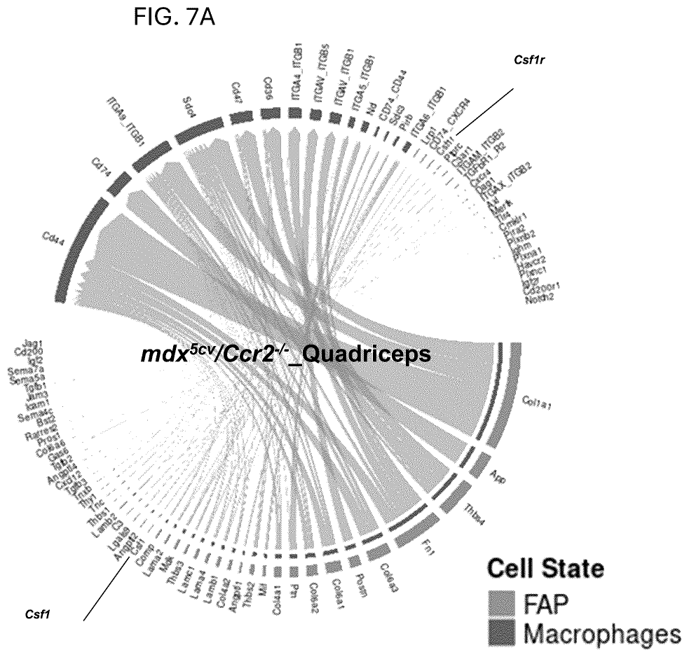

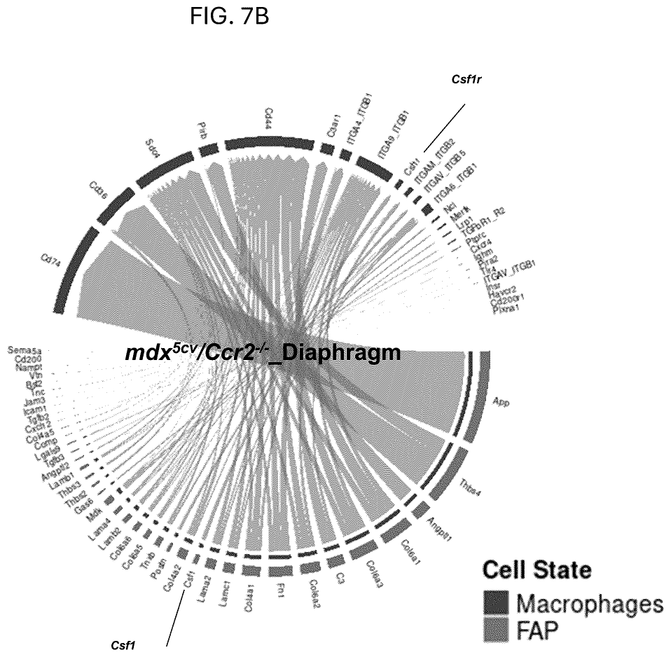

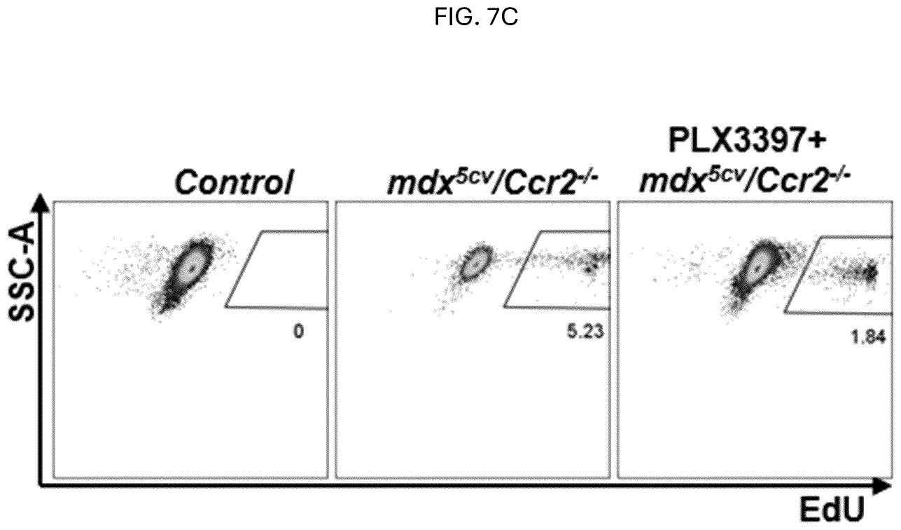

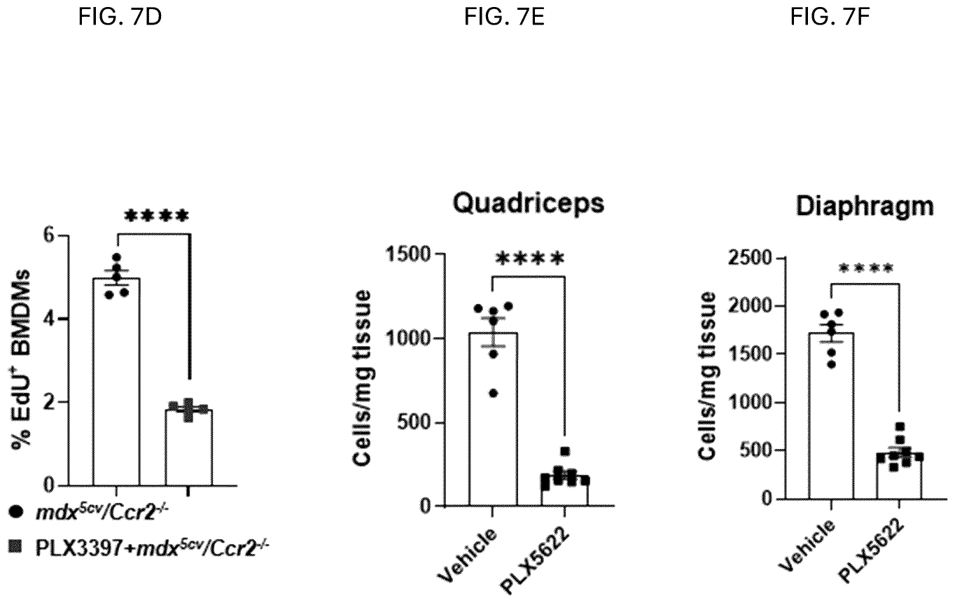

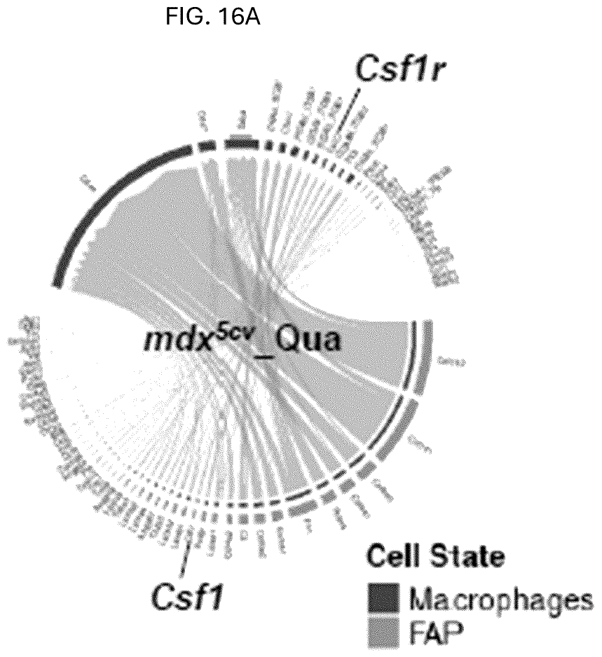

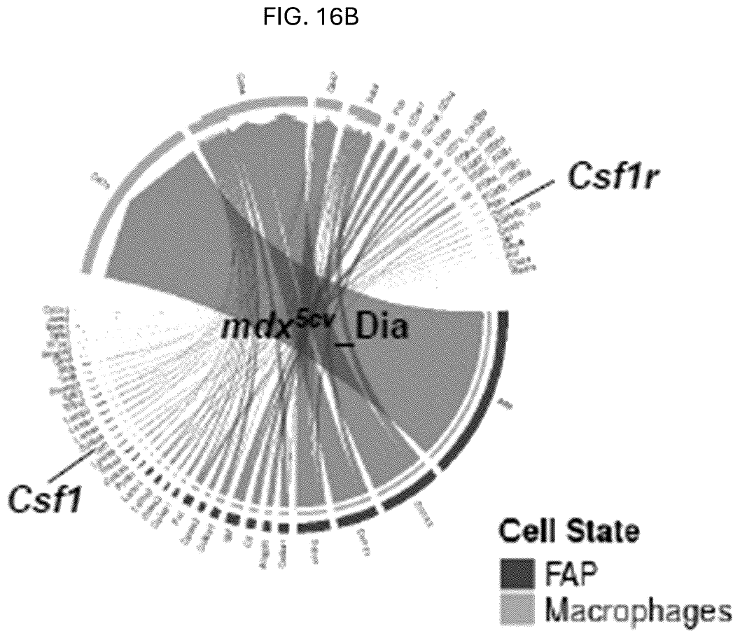

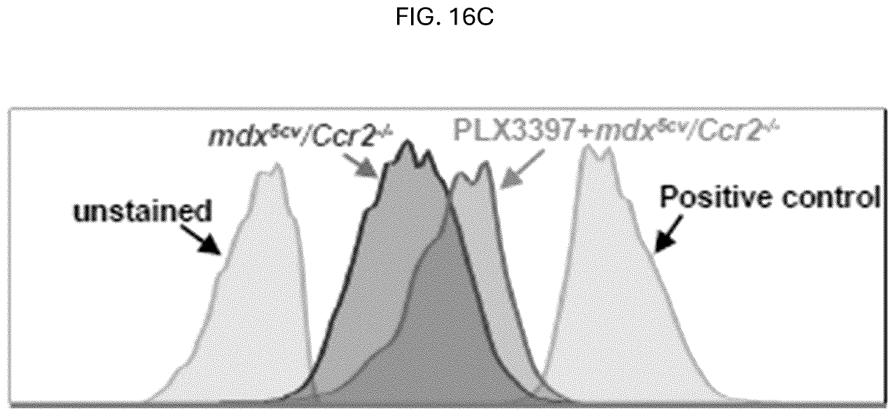

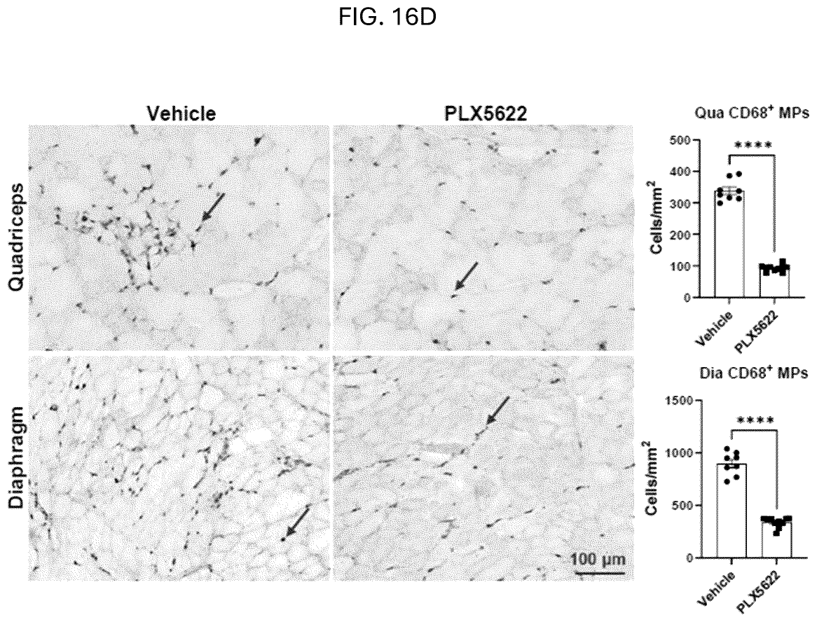

To further address whether FAPs could stimulate macrophage proliferation via CSF-1/CSF-1R signaling in dystrophic muscles, we first performed interactome ligand-receptor analysis of our scRNAseq data. FAPs did interact with macrophages via CSF-1/CSF-1R signaling in dystrophic muscles (FIGS. 7A-B, and FIGS. 16A-B). FIGS. 7A-7F show FAPs in mdx^{5cv}/Ccr2^{-/-} quadriceps and diaphragm stimulate macrophage proliferation via CSF-1/CSF-1R signaling. FIG. 7A and FIG. 7B show circos plots of scRNAseq data showing well-knowing ligand-receptor pairs between FAPs (sender) and macrophages (receiver) in mdx^{5cv}/Ccr2^{-/-} quadriceps (FIG. 7A) and diaphragm (FIG. 7B) at 14 weeks of age. FAPs communicate with macrophages through CSF-1/CSF-1R pairing. FIG. 7C and FIG. 7D show FACS analysis of EdU incorporation showing proliferation of WT bone marrow-derived macrophages (BMDMs) stimulated by the conditioned medium of mdx^{5cv}/Ccr2^{-/-} FAPs in the absence or presence of CSF-1R inhibitor PLX3397. BMDMs cultured in culture medium without conditioned medium of mdx^{5cv}/Ccr2^{-/-} FAPs and PLX3397 were used as control. (C) Dot plots represent results with FAP-conditioned medium prepared from 5 individual mice (N=5). FIG. 7D shows bar graph showing quantified data of (FIG. 7C). ****p<0.0001. FIG. 7E & FIG. 7F shows bar graph showing densities of intramuscular Ly6C^{lo} macrophages quantified by flow cytometry in the quadriceps (FIG. 7E) and diaphragm (FIG. 7F) of mdx^{5cv}/Ccr2^{-/-} mice receiving PLX5622 or vehicle control. N≥6 mice/group. ****p<0.0001. FIGS. 16A to 16B show Circos plots showing well-knowing ligand-receptor pairs of FAPs (sender) and macrophages (receiver) in mdx^{5cv} quadriceps (FIG. 16A) and diaphragm (FIG. 16B) muscles at 14 weeks of age. The FAPs communicate with macrophages through CSF-1/CSF-1R signal pathway. FIG. 16C shows FACS analysis of CSFE dilution showing proliferation of WT bone marrow derived macrophages (BMDMs) stimulated by the conditioned medium of mdx^{5cv}/Ccr2^{-/-} FAPs in the absence or presence of CSF-1R inhibitor PLX3397. Data represents results with FAP-conditioned medium prepared from 5 individual mice (N=5). FIG. 16D Immunostaining of CD68 using cryo-sections of quadriceps (Qua) and diaphragm (Dia) from mdx^{5cv}/Ccr2^{-/-} mice treated with PLX5622 or Vehicle control to determine the density of intramuscular macrophages. Sample pictures (Left) showing positive staining (as exampled by red arrows). Quantified data is shown as dot plots (Right). N≥8. ****p<0.0001. We then cultured bone marrow-derived macrophages (BMDMs) from WT mice with conditioned medium collected from in vitro-cultured FAPs isolated from mdx^{5cv}/Ccr2^{-/-} limb muscles, in the presence or absence of CSF-1R inhibitor, PLX3397 (46). The proliferation rate of BMDMs was then determined by both EdU incorporation (FIGS. 7C-D) and CSFE dilution assays (FIG. 16C). Compared to the culture medium-only control, addition of mdx^{5cv}/Ccr2^{-/-} FAP-conditioned medium in culture stimulated BMDMs with increased percentage of EdU⁺ cells, and this effect was inhibited by PLX3397 (FIGS. 7C-D). CSFE dilution assay further showed that the mdx^{5cv}/Ccr2^{-/-} FAP-conditioned medium stimulated multiple-cycles of division of BMDMs, which was inhibited by PLX3397 (FIG. 16C). We next determined the in vivo role of CSF-1R signaling in the expansion of intramuscular Ly6C^{lo} macrophages in mdx^{5cv}/Ccr2^{-/-} mice by treating the mice for 6 weeks with PLX5622, an oral CSF-1R inhibitor which is more selective than PLX3397 (47). The density of intramuscular macrophages decreased significantly in mice received PLX5622 than in mice received vehicle solution as assessed by FACS analysis (FIGS. 7E-F) and immunostaining (FIG. 16D), indicating a requirement of CSF-1R signaling for the expansion of Ly6C^{lo} macrophages in mdx^{5cv}/Ccr2^{-/-} mice. These findings together strongly support the hypothesis that FAPs stimulate intramuscular macrophage proliferation via CSF-1/CSF-1R signaling.

### Discussion

Chronic inflammation is one of the major muscle pathological features in DMD patients and animal models (1, 2). It is predominated by macrophage infiltration (9, 48). Although macrophages play an essential role in supporting regenerative repair of acute muscle injury (18, 19), they are largely pathogenic in chronic muscle injury associated with muscular dystrophy (8, 9, 13). A number of studies have shown that macrophages can increase muscle damage, promote muscle fibrosis, and impair muscle regeneration in mdx (8, 9, 12). Targeting the infiltration and pathogenic activation of macrophages has thus become a focus of DMD research (12, 13). Blocking infiltration of Ly6C^{hi} inflammatory monocytes and macrophages indeed improved muscle pathology and function in mdx and mdx^{5cv} mice, but the effect was transient and lost after the expansion of Ly6C^{lo} macrophages (9). Therefore, the intramuscular Ly6C^{lo} macrophages might also contribute to the progression of diaphragm fibrosis and dysfunction (9). By combining studies using Nur77 deficient mice, Flt3^{cre}/Rosa26^{LSL-YFP}-based lineage tracing of macrophage origins, and scRNAseq analysis, our present study has explored the mechanisms underlying the Ly6C^{lo} macrophage expansion in mdx^{5cv}/Ccr2^{-/-} mice and generated several novel and significant findings, which broaden the understanding of macrophages, especially resident macrophages, in muscular dystrophy.

First, our study shows no evidence of blood Ly6C^{lo} monocyte recruitment by dystrophic muscles in mdx^{5cv} mice, either with or without CCR2 deficiency. Nur77 deficiency, which results in diminished blood Ly6C^{lo} monocytes, does not affect Ly6C^{hi} or Ly6C^{lo} macrophage density in mdx^{5cv}/Nur77^{-/-} or mdx^{5cv}/Ccr2^{-/-}/Nur7^{-/-} muscles as compared to mdx^{5cv} or mdx^{5cv}/Ccr2^{-/-} controls. Likewise, blood Ly6C^{lo} monocytes are not recruited by acutely injured skeletal muscle either (23). Therefore, similar to those in acutely injured skeletal muscle, the infiltrating macrophages in chronically injured muscles in mdx are solely derived from Ly6C^{hi} inflammatory monocytes. Whether this is a generalized phenomenon that applies to other muscle diseases or injuries needs to be further addressed in the future.

Second, skeletal muscle resident macrophages undergo expansion in dystrophic muscles when Ly6C^{hi} monocyte/macrophage infiltration is blocked. Skeletal muscle resident macrophages have multiple origins, including prenatal yolk-sac primitive macrophages and fetal monocytes, as well as postnatal adult monocytes (24, 27). Resident macrophages derived from adult monocytes and a part of fetal monocytes are originated from adult bone marrow HSCs and prenatal HSCs, respectively, which can be identified as YFP⁺ cells by the Flt3^{Cre}/Rosa26^{LS-YFP} lineage tracing system (24). The YFP⁻ macrophages, identified by this system, are of non-HSC origin and purely derived from embryo (24). Our study clearly demonstrates the expansion of YFP⁻ macrophages in mdx^{5cv}/Ccr2^{-/-} but not mdx^{5cv} muscles (FIG. 2), which indicates that the YFP⁻ resident macrophages expand only when the Ly6C^{hi} monocyte/macrophage infiltration is blocked. The YFP⁺ macrophages are also mildly increased in mdx^{5cv}/Ccr2^{-/-} mice as compared to wild-type controls, but it is uncertain whether this is due to a low level of blood monocyte recruitment or YFP⁺ resident macrophage expansion. Nevertheless, there is expansion of resident macrophages, at least the YFP subpopulation, which is further confirmed by our scRNAseq analysis.

Three clusters of resident macrophages have been identified in steady-state murine tissues, correlating with different origins (12, 30, 49). The TLF⁺CCR2⁻MHCII^{lo}Ly6C^{lo} cluster consists of self-renewal macrophages originated from yolk sac and fetal monocyte precursors. The TLF⁻CCR2⁺MHCII^{hi}Ly6C^{lo} cluster can be entirely replaced by blood monocytes. The TLF⁻CCR2⁻MHCII^{hi}Ly6C^{lo} can be renewed by proliferation and replenished by blood monocytes, and they can be originated from embryonic and adult bone marrow hematopoiesis. Our scRNAseq analysis shows a significant increase in TLF⁺ resident-like macrophages in dystrophic muscles in mdx^{5cv}/Ccr2^{-/-} mice as compared with mdx^{5cv} controls, and these macrophages are TLF⁺CCR2⁻MHCII^{lo}Ly6C^{lo}, resembling the self-renewal, embryo-derived resident macrophages in steady-state tissues. Our lineage tracing analysis further shows the expansion of embryo-derived YFP-macrophages in mdx^{5cv}/Ccr2^{-/-} mice but not in mdx^{5cv} muscles. The findings provide solid evidence that the embryo-derived resident macrophages expand in mdx^{5cv} skeletal muscle when CCR2-mediated monocyte/macrophage infiltration is blocked. This expansion of pre-existing resident macrophages is achieved by increased proliferation not decreased apoptosis.

Third, FAPs appear the main regulators of intramuscular resident macrophage expansion via their production of the important macrophage growth factor, CSF-1. FAPs play important roles in skeletal muscle homeostasis and injury repair (50-53). They accumulate in skeletal muscle of DMD patients and mdx mice (53, 54). The expression of CSF-1 is significantly increased in mdx^{5cv}/Ccr2^{-/-} muscles compared to mdx^{5cv} controls. Although both neutrophils and FAPs express CSF-1, FAPs greatly outnumber neutrophils in the dystrophic muscles, indicating that FAPs are the primary source of CSF-1. More importantly, the intramuscular density of FAPs is also significantly higher in mdx^{5cv}/Ccr2^{-/-} muscles than in mdx^{5cv} controls, of which the precise mechanisms need to be investigated in the future. Thus, although the CSF-1 expression by FAPs at a single-cell level is similar between mdx^{5cv}/Ccr2^{-/-} and mdx^{5cv} control, the CSF-1 expression at a whole muscle level is significantly higher in mdx^{5cv}/Ccr2^{-/-} muscles than in mdx^{5cv} controls. The potential role of FAPs in driving the expansion of intramuscular macrophages is further supported by the in vivo evidence of the interaction between FAPs and macrophages via CSF-1/CSF-1R signaling and the in vitro ability of FAP-conditioned medium to stimulate BMDM proliferation in a CSF-1R signaling-dependent manner. Moreover, in vivo CSF-1R inhibition in mdx^{5cv}/Ccr2^{-/-} mice significantly blocked the expansion of intramuscular Ly6C^{lo} macrophages. The findings are consistent with a published study showing that a long-term inhibition of CSF-1R in mdx mice depletes both TIM4⁻ and TIM4⁺ resident macrophages in skeletal muscle (12). The interaction between FAPs and resident macrophages through CSF-1/CSF-1R signaling is also present and important in steady-state muscle, as a recent published study demonstrates that the FAP-derived CSF-1 is required for the survival of muscle resident macrophages regardless of their origins, and that a subset of DPPIV⁺ FAPs contributes to the niche of self-renewal resident macrophages (43). However, our data indicate that, in dystrophic muscles, the Csfl expression is contributed by all FAP clusters, with the highest contribution from the pro-inflammatory cluster whose fraction is much higher in dystrophic muscles than that in steady-state muscles (44). The difference in the relative contribution to Csfl expression by different FAP clusters is likely resulted from the difference in FAP activation and differentiation status determined by the different intramuscular microenvironment between steady-state and dystrophic muscles.

Fourth, our study strongly suggests that skeletal muscle resident macrophages undergo pathogenic activation in dystrophic muscles in the absence of CCR2-mediated macrophage infiltration. Infiltrating macrophages are overall pathogenic in mdx muscles, as blocking macrophage infiltration reduces muscle fibrosis, increases muscle regeneration, and improves muscle function (8, 9). On the other hand, the role of self-renewal resident macrophages in both acute and chronic skeletal muscle injuries are largely unexplored (13, 27, 55). It has been reported that the activated CD9⁺TREM2⁺SPP1⁺ macrophages that express a high level of Gpnmb, Fabp5 and Cd63 are enriched in fibrotic tissues and they play a profibrotic role (31-33). We have identified similar macrophage subtypes (Spp1⁺ and Trem2⁺) in the dystrophic muscles, whose fractions increase dramatically in both mdx^{5cv} and mdx^{5cv}/Ccr2^{-/-} muscles compared to WT controls. The findings are consistent with a recent study published by Coulis et al. showing an increased accumulation of a gal-3⁺ macrophage subtype that features a high-level expression of Spp1, Fabp5, Gpnmb, Trem2, Lgals3 (gal-3), and various cathepsin genes in mdx mice at the early acute phase of the disease (4 to 6 weeks of age) (56). The authors also showed evidence of interaction between gal-3⁺ macrophages and FAPs through SPP1 signaling, which stimulated the pro-fibrotic activation of FAPs in mdx muscles (56). The association between Spp/expression and muscle dystrophy was also supported by another study in DMD mouse models (57). These findings indicate a pro-fibrotic activation of intramuscular macrophages in dystrophic muscles, which contributes to the pathological progression of muscular dystrophy. Importantly, our study showed a similar expansion and fraction of Spp1⁺/Trem2⁺ macrophages in mdx^{5cv}/Ccr2^{-/-} and mdx^{5cv} muscles, which strongly suggests that the resident macrophages in mdx^{5cv}/Ccr2^{-/-} muscles also undergo pathogenic activation, similar to the infiltrating macrophages in mdx^{5cv} muscles. This pathogenic activation is most likely triggered by the tissue microenvironment. The notion is supported by a previous report by Coulis et al. showing the activation of both monocytes and muscle resident macrophages from healthy WT mice into gal-3⁺ phenotype after being adoptively transferred into mdx muscle (56). Therefore, the activation and functional status of intramuscular macrophages is determined more likely by the tissue microenvironment than by the macrophage origins. It is important to determine the key environmental factors driving such pathogenic activation of macrophages for therapeutic intervention. Hoeft et al. reported that loss of chemokine CXCL4 abrogated differentiation of profibrotic Spp1⁺ macrophages and ameliorated fibrosis in injured heart and kidney, and that platelets induced a profibrotic Spp1⁺ macrophage signature via CXCL4 (32). It will be interesting to study whether CXCL4 and platelets also play a role in the pathogenic activation of macrophages in chronically injured muscles of mdx^{5cv} and mdx^{5cv}/Ccr2^{-/-} mice. The expansion and pathogenic activation of pre-existing resident macrophages likely contribute to the progression of muscle dystrophy in mdx^{5cv}/Ccr2^{-/-} mice at late stage.

In summary, our study has characterized the origins and activation of intramuscular Ly6C^{lo} macrophages in mdx^{5cv}/Ccr2^{-/-} mice. Our findings indicate that the self-renewal skeletal muscle resident macrophages expand to compensate for the lack of inflammatory macrophage infiltration in mdx^{5cv}/Ccr2^{-/-} mice, and these resident macrophages undergo pathogenic activation. FAPs may play an important role in driving resident macrophage expansion via their CSF-1 production. Targeting the expansion and pathogenic activation of resident macrophages may represent an important approach of macrophage-based therapies for muscular dystrophy, in addition to inhibiting the infiltration of inflammatory monocytes and macrophages.

### Example 3. Materials and Methods

### Materials and Methods

Detailed animal information of strains, genetic background, and sources, can be found herein. Breeding strategies are described below. Our study protocols were approved by the Institutional Animal Care and Use Committee at the Hospital for Special Surgery (New York, NY, USA.) and Boston University School of Medicine (Boston, MA, USA.). Quadriceps and diaphragm muscles were collected for immunostaining, qRT-PCR, ELISA, and single-cell based analyses including FACS, proliferation, apoptosis, and scRNAseq. Blood was collected for analyzing monocytes. For in vitro culture experiments, FAPs were isolated from mdx^{5cv}/Ccr2^{-/-} muscles to prepare conditioned media, and BMDMs were prepared from the bone marrow of WT mice. Detailed protocols of all experimental procedures, and the methods of scRNAseq data processing and analysis, are described in details below.

### Data Availability

The datasets produced in this study are available in the following databases:
RNA-Seq data: Gene Expression Omnibus GSE265803
   (ncbi.nlm.nih.gov/geo/query/acc.cgi?acc=GSE225803).

### Reagents and Tools

All reagents and tools are listed in Table S9.

### Animals

mdx^{5cv}, Nur77^{-/-} and Rosa26^{LSL-YFP} mice were purchased from The Jackson Laboratory (Bar Harbor, ME, USA). Flt3Cre transgenic mice were kindly provided by Dr. Kory Levin (Washington University School of Medicine, St. Louis, MO, USA). The Ccr2^{-/-} mice were originally kindly provided by Dr. Israel Charo, which were backcrossed nine times with the C57BL/6J mice as previously described. Because the Flt3Cre transgene located on the Y chromosome, male Flt3Cre mice were crossed with female Rosa26LSL-YFP mice to generate Flt3Cre-Rosa26LSL-YFP mice. Similarly, male Flt3Cre/Rosa26LSL-YFP mice were crossed with female Ccr2−/− mice to obtain Flt3Cre/Rosa26LSL-YFP/Ccr2+/− mice, which were further crossed with female Ccr2−/− mice to generate Flt3Cre/Rosa26LSL-YFP/Ccr2−/− mice. mdx5cv/Ccr2−/− mice were generated by crossbreeding mdx5cv mice with Ccr2−/− mice. mdx5cv/Nur77−/− mice were generated by crossbreeding mdx5cv mice with Nur77−/− mice. mdx5cv/Ccr2−/−/Nur77−/− mice were generated by crossbreeding mdx5cv/Ccr2−/− mice with mdx5cv/Nur77−/− mice. Only males were used in this study because DMD only affect males. PLX5622 (MedChemExpress, Monmouth Junction, NJ, USA) was used to inhibit CSF-1R in vivo as described previously (1). We treated 8-week-old mdx5cv/Ccr2−/− mice with daily 100 mg/kg bodyweight of PLX 5622 or vehicle solution by oral gavage until 14 weeks of age. Our study protocols were approved by the Institutional Animal Care and Use Committee at the Boston University School of Medicine (Boston, MA, USA.).

### Blood Collection, Red Blood Cell Lysis, and Single-Cell Suspension Preparation for FACS Analysis

Mouse peripheral blood was collected from the heart of anesthetized mice. 200 μl of blood/mouse was centrifuged at 500×g for 3 minutes. Blood serum was removed. Cell pellet was then re-suspended in 3 ml of 1×ACK Lysis Buffer (Millipore Sigma, Burlington, MA, USA) in a 15-ml centrifuge tube for 3 minutes. 12 ml of 1×phosphate-buffered saline (PBS, pH 7.4) was then added, mixed thoroughly, and centrifuged at 300×g, 4° C. for 10 minutes. Supernatant was then discarded. The cell pellet was washed by re-suspension with 1×PBS (pH 7.4) and centrifuged at 300×g, 4° C. for 10 minutes. Supernatant was discarded and cell pellet was re-suspended in ice-cold FACS buffer (1×PBS (pH 7.4), 0.5% (w/v) bovine serum albumin (BSA, Millipore Sigma), 0.1% (w/v) sodium azide) for the following FACS analysis.

### Muscle Sample Collection

Quadriceps and diaphragm were collected from 14-week-old male WT mice, and 14-week-old and 24-week-old male mdx5cv mice. Tendons were removed from the tissue specimens. Collected muscle samples were thoroughly washed in 1×PBS (pH 7.4) to remove blood contamination.

### Immunostaining for Identifying Intramuscular Macrophages

Collected muscle samples were frozen in liquid nitrogen-chilled isopentane for 30 seconds. Cross sections were then obtained on a Leica cryostat at a thickness of 10 μm. Sections were air-dried for 30 minutes at room temperature and then fixed in 4% (w/v) of paraformaldehyde (Thermo Scientific) for 10 minutes followed by washing in 1×PBS for 5 minutes. Immunostaining of intramuscular macrophages were then performed with anti-CD68 antibody (FA-11, Bio-Rad, Hercules, CA, USA) as described previously (2). Briefly, sections were blocked with 10% Normal Goat Serum in PBS-T (1×PBS with 0.2% Triton X-100) for 2 hours at room temperature, followed by incubation with anti-CD68 antibody (1:400 diluted in 10% Normal Goat Serum in PBS-T) overnight at 4° C. Sections were then washed with PBS-T (3 times with 5 minutes each), and incubated with Biotinylated Rabbit anti-Rat secondary antibody (Vector Laboratories, Newark, CA, USA. 1:200 diluted in 10% Normal Goat Serum in PBS-T) at room temperature for 2 hours. After being washed with PBS-T, the sections were incubated in ABC reagent (Vector Laboratories) for 20 minutes. Following PBS-T washing, the sections were developed with DAP Substrate Kit (Vector Laboratories) following manufacturer's instruction.

### Muscle Single-Cell Suspension Preparation

Muscle single-cell suspension was prepared by collagenase/dispase digestion. Briefly, each muscle was minced in 2.5 ml of digestion solution containing 1 U/ml of collagenase B and 1 U/ml of dispase II (Roche Diagnostics, Indianapolis, IN, USA) in PBS and incubated at 37° C. for 1 hour. The reaction was terminated by addition of 10 ml of PBS with 10% of fetal bovine serum (FBS, Corning, Glendale, AZ, USA). The mixture was then filtered through 70-μm cell strainer and subjected to centrifugation at 250×g for 5 min. The pellet was collected, and the supernatant was centrifuged again at 250×g for 5 minutes at 4° C. The pellets were combined, washed with PBS, and centrifuged at 670×g for 10 min. The pellet was resuspended in 3 ml of PBS, filtered through a 40-μm cell strainer, layered on an equal volume of Lymphocyte-M solution (Cedarlane, Burlington, NC, USA), and centrifuged at 2100×g for 45 min. Cells at the interface were collected in 10 ml PBS containing 10% FBS, centrifuged at 670×g for 10 minutes at 4° C., and re-suspended in FACS buffer.

### Spleen Single-Cell Suspension Preparation for FACS Analysis

Mouse spleen was collected following euthanasia. Each spleen was cut into pieces in 1 ml of 1×cold PBS (pH 7.4), grinded through a 40-μm cell strainer. The passthrough was centrifuged at 500×g for 3 minutes. Supernatant was removed. Cell pellet was then re-suspended in 3 ml of 1×ACK Lysis Buffer (Millipore Sigma, Burlington, MA, USA) in a 15-ml centrifuge tube for 3 minutes. 12 ml of 1×PBS (pH 7.4) was then added, mixed thoroughly, and centrifuged at 300×g, 4° C. for 10 minutes. Supernatant was then discarded. The cell pellet was washed by re-suspension with 1×PBS (pH 7.4) and centrifuged at 300×g, 4° C. for 10 minutes. Supernatant was discarded and cell pellet was re-suspended in ice-cold FACS buffer (1×PBS (pH 7.4), 0.5% (w/v) bovine serum albumin (BSA, Millipore Sigma), 0.1% (w/v) sodium azide) for the following FACS analysis.

### Flow Cytometry Analysis and Cell Sorting

Single-cell pellets were washed once in ice-cold FACS buffer (1×PBS, 0.5% (w/v) bovine serum albumin (BSA, Millipore Sigma), 0.1% (w/v) sodium azide), centrifuged at 500 g for 3 minutes at 4° C., and re-suspended in ice-cold FACS staining buffer (1×PBS, 2% (w/v) BSA, 2% (v/v) normal mouse serum (Millipore Sigma).

For FACS analysis of different cell types, cells were stained on ice for 20 minutes with fluorescence-labelled antibodies targeting cell-type specific markers. For intracellular staining of CSF-1, permeabilization and staining were performed with CytoFix/CytoPerm reagents from BD Bioscience (San Jose, CA, USA) following manufacturer's instructions. Blood monocytes were identified as CD45+CD115+Ly6Chi/lo cells. Macrophages were identified as CD45+CD64+F4/80+cells. YFP+macrophages were identified as CD45+CD64+F4/80+YFP+cells. YFP-macrophages were identified as CD45+CD64+F4/80+YFP-cells. FAPs were identified as Sca-1+PDGFRα+CD45-CD31-α7-integrin-cells. Spleen T cells were identified as CD45+CD3+cells. Fluorescence-labelled corresponding normal IgG isotypes were included as negative controls for gating. All antibodies were from commercial providers (See Key Resources Table for details) and diluted for staining following manufacturer's recommendations. After staining, cells were washed twice with FACS buffer and analyzed on an LSR II (BD Bioscience, San Jose, CA, USA) with BD FACS Diva™ software. Collected data were then analyzed using FlowJo software (Tree Star, Inc., Ashland, OR, USA).

To clean up single cell suspension for subsequent single-cell based analysis, cell sorting based on FSC/SSC was performed to exclude dead cells, cell duplexes, and tissue debris. Sorting macrophages, non-macrophages, FAPs and non-FAPs for qRT-PCR analysis were performed with antibodies listed above. Cell sorting was done by the Flow Cytometry Core of Boston University School of Medicine. Collected cells from 5 individual male mice were combined and pelleted by centrifuging at 300×g for 10 minutes. For scRNAseq analysis, pellets were re-suspended in FBS supplemented with 10% of DMSO and cryopreserved in liquid nitrogen. For qRT-PCR analysis, pellets were dissolved in TRIzol reagent (Invitrogen, Waltham, MA, USA) for RNA preparation.

### L-929 Cells-Conditioned Media

L-929 cells were grown for three passages from cryogenic storage before seeding for media collection. Cells were seeded in high glucose DMEM containing 10% (vol/vol) fetal bovine serum (FBS), 1 mM L-glutamine, 100 U/ml penicillin, and 100 μg/ml streptomycin at a density of ~6,500 cells per cm2 of available surface area in T-75 flasks (15 ml/flask). The medium was carefully removed after 7 days of culture and replaced with fresh DMEM media for a subsequent 7 days. The two supernatant collections were then combined and sterile filtered before aliquoting into 50-ml falcon tubes and stored at −80° C.

### Bone Marrow-Derived Macrophages (BMDMs) Isolation and Culture

Bone marrow cells were collected from the tibias and femurs of 14-week-old C57BL/6J mice. Red blood cells were removed using ACK lysis buffer (Millipore Sigma) as described in the previous section of “Blood collection, red blood cell lysis, and single-cell suspension preparationfor FACS analysis”. To obtain BMDMs, bone marrow cells was then cultured in DMEM supplemented with 10% (vol/vol) FBS, 10 U/mL penicillin, 100 mg/mL streptomycin, 170 100 μM 2-ME, and 30% L-929 cells-conditioned media, in 6-well plate at a density of 106 cells/well. After 6 days, non-adherent cells were discarded along with the supernatant. The adherent cells were cultured in fresh DMEM media with 10% (vol/vol) FBS, 10 U/mL penicillin and 100 mg/mL streptomycin.

### Apoptosis Assay

Cells undergoing apoptosis display an increase in nuclear chromatin condensation which makes cell-permeable nucleic acid stain become hyperfluorescent, thus enabling the identification of apoptotic cells when combined with a cell-impermeable nucleic acid stain to exclude dead cells. Here, we used cell-permeable Vybrant™ DyeCycle™ Violet dye in combination with cell impermeable TO-PROTM3 dye to detect apoptotic cells (Vybrant+ TO-PROTM3-). Muscle single cell suspension was first stained with antibodies identifying macrophages as described in the section of flow cytometry analysis and cell sorting. Apoptosis of BMDMs was induced in vitro by treatment of actinomycin D (0.5 □M) for 12 hours (3) or by CSF-1 starvation (removal of L-929 cells-conditioned media) for 24 hours, compared to un-treated control. Peripheral mature T lymphocytes are known to contain a significant portion of apoptotic cells (4), and they were thus isolated from spleen and assayed simultaneously as in vivo positive controls. After being washed twice with FACS buffer, cells were again resuspended in FACS buffer and stained with Vybrant™ DyeCycle™ Violet/TO-PROTM3 Ready Flow™ reagents (Invitrogen) following manufacturer's instruction. Stained cells were analyzed by flow cytometry immediately.

### In Vivo Macrophage Proliferation 193 Assay

mdx5cv, mdx5cv/Ccr2−/−, mdx5cv/Nur77−/− and mdx5cv/Ccr2−/−/Nur77−/− mice at 14 weeks of age received intraperitoneal injection of EdU (100 mg/kg, Baseclick GmbH, Neuried, Germany). Quadriceps and diaphragm were then collected 24 hours later, and single-cell suspension was prepared as described in the section of muscle single-cell suspension preparation. Macrophage proliferation was then analyzed by determining the incorporation of EdU using EdU Click 647 Kit (Baseclick GmbH) following manufacturer's instruction, combined with FACS staining and analysis of macrophages as described in the section of flow cytometry analysis and cell sorting.

### Elisa Assay

Quadriceps and diaphragm were washed with PBS and snap frozen. Following tissue homogenization in PBS with complete protease inhibitor cocktail (Roche Diagnostic) in a Bead Mill 24 homogenizer (Fisher Scientific, Hampton, NH, USA), samples underwent 3 freeze-thaw cycles and supernatant was collected after centrifugation (10,000×g for 5 min at 4° C.). Mouse anti-CSF-1 ELISA kit (R & D Biosystems), Mouse anti-CTGF ELISA kit (R & D Biosystems, Minneapolis, MN, USA) and mouse anti-PDGFa ELISA kit (Thermo Fisher Scientific) was used on the supernatant following manufacturer's instruction. mRNA preparation and quantitative real-time PCR (qRT-PCR) Total RNA samples were prepared using TRIzol reagent (Invitrogen). Samples 215 of macrophages and other cell types were prepared by cell sorting as stated above and dissolved in TRIzol reagent. To prepare whole-muscle RNA, quadriceps, or diaphragm, collected from 14-week-old male mice, were homogenized in TRIzol reagent using Bead Mill 24 homogenizer (Fisher Scientific). Total RNA was then prepared following manufacturer's instruction. TRIzol-prepared total RNA was further cleaned up using RNeasy Micro Kit (Qiagen, Germantown, MD, USA) following manufacturer's instruction. Concentration of cleaned RNA was measured using NanoDrop 2000 Spectrophotometer (Thermo Scientific). lug of total RNA was then reverse transcribed into cDNA using SuperScript III Reverse Transcriptase system (Invitrogen) following manufacturer's instruction. Expression of genes at mRNA level was then analyzed by real-time PCR with PowerTrack™ SYBR Green Master Mix (Applied Biosystems, Waltham, MA, USA) using StepOne™ Real Time PCR System (Applied Biosystems). Sequences and providers of the primers used are listed in Key Resources Table. The sequences and target genes of the primers were listed in Key Resources Table. Data was calculated by ΔΔCt method. For sorted cell analysis, data was presented as bar graph with error bar showing standard deviation of triplicated PCR results. For whole muscle analysis, data were presented as scatter plot showing results of each individual samples.

### FAP Isolation and Culture, and Collection of FAP-Conditioned Medium

Isolation and culture of skeletal muscle FAPs were modified from a published study (5). Muscles from both hind limbs of 14-week-old mdx5cv/Ccr2−/− mice were isolated, and non-muscle tissue was removed before muscles were digested with digestion buffer. Muscles from 1 entire hindlimb were minced in 5 ml of digestion solution containing 1 U/ml of collagenase 238 B and 1 U/ml of dispase II (Roche Diagnostics) in PBS and incubated at 37° C. for 1 hour. The reaction was terminated by addition of 10 ml of PBS with 10% (vol/vol) FBS. The mixture was then filtered through 70-μm cell strainer and subjected to centrifugation at 250×g for 5 min. The pellet was collected, and the supernatant was centrifuged again at 250×g for 5 minutes at 4° C. The pellets were combined, washed with DMEM containing 10% (vol/vol) FBS, and centrifuged at 670×g for 10 min. Cells from the same mouse were resuspended in DMEM containing 10% (vol/vol) FBS, combined, and cultured in 10-cm tissue culture dish at 37° C., 5% CO2 for 90 minutes. Supernatant and floating cells were then discarded. The adherent cells were maintained in DMEM with 10% (vol/vol) FBS, and grown at 37° C., 5% CO2 into confluency. Supernatant was then collected, sterile filtered, and stored at −80° C. FAPs from each individual mouse were9 cultured separately to collect conditioned medium.

BMDM Stimulation with FAP-Conditioned Medium and in Vitro Proliferation Assay

BMDMs were cultured in DMEM containing 10% FBS, 30% FAP-conditioned medium, and 20 μM EdU in absent or present of 100 nM PLX 3397 for 24 hours. BMDMs cultured in DMEM containing 10% FBS and 20 μM EdU without PXL 3397 were used as controls. Macrophages were detached using cell stripper, pelleted by centrifugation at 300×g for 10 min, resuspended in FACS buffer. EdU staining was carried out using the EdU Click 647 Kit (BaseclickGmbH, Germany) following manufacturer's instruction. The stained macrophages were analyzed on an LSR II (BD Bioscience, San Jose, CA, USA) with BD FACS Diva™ software. Data analysis was carried out using FlowJo software.

### BMDM CFSE Dilution Assay

BMDMs were detached using cell stripper, pelleted by centrifugation at 300×g for 10 min, and resuspended in 1 μM CFSE solution (Thermo Fisher Scientific) for 15 min at what temperature 37° C. Then the macrophages were washed with DMEM containing 10% FBS to remove the non-incorporated dye. The cells were plated to the 12-well plate (5×105/well) in DMEM containing 20% FBS, 30% FAPs supernatant in absent or present of 100 nM PLX 3397 (6). After 3 days, the macrophages were detached from the plate, fixed with 4% PFA, resuspended in FACS buffer, and subjected to FACS analysis with LSR II. Data analysis was carried out using FlowJo software.

Single-Cell cDNA Library Preparation and Sequencing

Mouse skeletal muscle cell suspensions with viabilities between 74.4-87.6% were run on 10X genomics chip at concentrations varying between 810,000-1,280,000 cells/mL for a targeted cell recovery of 10,000 cells per sample. This was done using the 10X genomics 3′v3.1 Dual index kit. Following the 10X Genomics Chromium controller run the Gel-beads in emulsion (GEMs) were transferred to strip tubes and subsequently put into a thermal cycler using the following parameters: 45 min at 53° C., 5 min at 85° C., hold at 4° C. The samples were then stored in a −20° C. freezer overnight. The next day samples were thawed at room temperature before adding the recovery agent to break the GEMs. Subsequently the cDNA was purified then amplified in preparation for gene expression library preparation. Next the gene expression libraries were generated by following 10X Genomics library preparation protocol and the size, Sensitivity DNA Assay (Agilent Technologies, Lexington, MA, USA). The libraries were then pooled to 5 nM and sequenced on an Illumina NextSeq 2000 instrument at an 850 pM input and 2% PhiX spike in using a P3 100 cycle flow cell (Illumina, San Diego, CA, USA) resulting in over 25,000 reads per cell.

### Preprocessing and Quality Control of Single-Cell Data

The 10X CellRanger tool was used for demultiplexing, alignment, identification of cells, and counting of unique molecular indices (UMIs). Specifically, the CellRanger mkfastq command was used to demultiplex raw base call (BCL) files generated by Illumina sequencers into FASTQ files. The CellRanger count command was used to perform alignment and create UMI count matrices using parameters-expect-cells=10,000. Droplets with at least 500 UMIs underwent further quality control with the SCTK-QC pipeline 1. The median number of UMIs was 6,627, the median number of genes detected was 2,234, the median percentage of mitochondrial reads was 3.37%. Cells with less than 500 counts, less than 500 genes detected, or more than 10% mitochondrial counts were excluded leaving a total of 28,888 cells for the downstream analysis.

### Clustering of Single-cell Data With Celda

The celda package was used to bi-clustering genes into modules and cells into subpopulations. The 5,000 most variable features were selected using the seuratFindHVG function in the singleCellTK package, excluding features with less than 3 counts in 3 cells. The recursiveSplitModule and recursiveSplitCell functions were used to select the model with 18 modules and 60 cell subpopulations after examining the Rate of Perplexity Change (RPC). Cells were embedded in two dimensions with UMAP using the celdaUmap function. Heatmaps for specific modules were generated using the moduleHeatmap function. Markers for each cluster were identified with the findMarkerDiffExp function from the singleCellTK package with a threshold of FDR<0.05.

### Subclustering of Macrophages, FAPs, and Satellite Cells

The Seurat package was used to cluster the subsets of macrophages, FAPs, and satellite cells. The feature expression was normalized using the SCTransform function with version v2. Linear dimensional reduction PCA was performed on variable genes using the RunPCA function, and cells were embedded in two-dimensional space using the RunUMAP function. Cell clusters were identified using the FindClusters function, and differentially expressed genes were computed using the FindAllMarkers function with the Wilcoxon rank sum test method and default parameters. Top marker genes in each cluster shown in Heatmap were ordered by log fold-change of the average expression.

### Volcano Plots of Differentially Expressed Genes

Differential gene expression among macrophages were determined using the runDEAnalysis function from the singleCellTK package, using a Wilcoxon rank sum test. Comparisons were conducted between mdx5cv and mdx5cv/Ccr2^{-/-} in both quadriceps and diaphragm muscle samples. The differentially expressed genes were visualized with the EnhancedVolcano package, using FDR<0.05 and log fold-change>0.5 as thresholds for significance.

### Ligand-Receptor Pair Analysis

The CellChat package in R was used to infer and analyze cell-cell communication probability. The CellChatDB database was selected for cell-cell communication analysis. Over-expressed ligand-receptor pairs were identified using the identifyOverExpresesedInteractions function, and the cellular communication network was inferred using the computeCommunProb with the triMean method and 25% truncated mean. Significantly enriched pathways were identified with the computeCommunProbPathway function. The significantly enriched pathways were then visualized with chord plots with the netVisual_individual and netVisual_aggregate functions, and bubble plots were created with the netVisual_bubble function. The dominant senders, receivers, mediators, and influencers were identified for each cell group using the netAnalysis_computeCentrality function and visualized in heatmaps to show dominant incoming and outgoing signaling in the cell groups.

### Quantification and Statistical Analysis

Data were analyzed using GraphPad Prism 10 software (GraphPad Software, San Diego, CA, USA). The Mann-Whitney test was performed to compare between two groups; the KruskalWallis test followed by Dunn's test was performed to compare multiple (23) groups. A p value of <0.05 was considered statistically significant.

### Example 4

Duchenne muscular dystrophy (DMD) is the most common genetic muscle disease with no effective cure currently. Muscle inflammation associated with DMD is predominated by inflammatory monocyte/macrophage infiltration, blockage of which reduces muscle damage and fibrosis, and improves muscle regeneration. However, the benefit is transient, alongside the expansion of intramuscular macrophages despite diminished monocyte/macrophage infiltration.

Blocking blood monocytes-derived macrophage infiltration via CCR2 inhibition does NOT persistently reduce the accumulation of intramuscular macrophages and improve the dystrophic phenotype of skeletal muscle in DMD mouse model. Intramuscular macrophages can originate from embryonic hematopoiesis or post-natal bone marrow hematopoiesis (27). Murine blood monocytes consist of two principal subsets, Ly6C^{hi} (CCR2+/CX3CR1^{low}) and Ly6C^{lo} (CCR2⁻/CX3CR1^{hi}) cells, with corresponding subsets in humans (16). Tissue recruitment of cells from blood circulation requires the chemokine system with the expression of chemokine ligands by tissue cells to chemoattract blood cells that express the corresponding chemokine receptors. CC chemokine receptor 2 (CCR2) and its ligands play a critical role in mediating tissue recruitment of Ly6C^{hi} inflammatory monocytes (17-20), which differentiate into Ly6C^{hi} inflammatory macrophages within injured tissues (16) and then switch into Ly6C^{lo} macrophages (21). Blood Ly6C^{lo} monocytes patrol the vascular endothelial surface at the steady state, and they may enter tissue via CX3CR1 to contribute to tissue resident macrophages (74). The critical role of CCR2 in mediating the recruitment of Ly6C^{hi} MOs by dystrophic muscles was confirmed by Mojumdar, et al. showing that genetic ablation or pharmacological inhibition of CCR2 blocked inflammatory macrophage infiltration at early stages (6 and 12 weeks) (8). It also improved muscle pathology and function in the mdx diaphragm at early stages. Our published study (9) further showed that the knockout of CCR2 in the mdx^{5cv}/Ccr2^{-/-} mice diminished intramuscular Ly6C^{hi} macrophages at all stages, but it reduced Ly6C^{lo} macrophages only at early stages (4 and 9 weeks) but not late stages (14 weeks or 6 months). CCR2 deficiency reduced diaphragm muscle damage and fibrosis, and improved diaphragm muscle regeneration and function at 14 weeks but not 6 months (9). Therefore, blocking Ly6C^{hi} monocyte/macrophage infiltration does not provide sustained improvement of mdx^{5cv} diaphragm dystrophy, and the beneficial effects from CCR2 deficiency are lost after the expansion of intramuscular Ly6C^{lo} macrophages.

Pre-existing skeletal muscle resident macrophages expand to compensate the loss of blood monocyte recruitment in absent of CCR2, contributing to the pathology of dystrophic muscles. We have confirmed in our recent submitted work (See attached “Wang et al_PNAS Manuscript” for details) that the Ly6C^{lo} macrophages in mdx^{5cv}/Ccr2^{-/-} muscles are also pathogenic, showing features of an identified pro-fibrotic macrophage subtype. Previous studies in multiple fibrosis models (31-33), including mdx mice (59), have reported a pathogenic pro-fibrotic macrophage subset featuring a high level of expression of pro-fibrotic genes, including Spp1 and Trem2. This macrophage subset is associated with the fibrotic area in mdx muscle, and stimulates the expression of collagens by fibrogenic cells (59). Our work identified a similar macrophage subset (Spp1⁺/Trem2⁺), of which the fraction increased dramatically in both mdx^{5cv} and mdx^{5cv}/Ccr2^{-/-} muscles compared to WT controls, despite the lack of Ly6C^{hi} monocyte/macrophage infiltration in mdx^{5cv}/Ccr2^{-/-} muscles. The findings indicate that the Ly6C^{lo} macrophages undergo pathogenic activation in mdx^{5cv}/Ccr2^{-/-} muscles. We further showed evidence that the Ly6C^{lo} macrophages in mdx^{5cv}/Ccr2^{-/-} muscles are not recruited from blood Ly6C^{lo} monocytes, but are instead from the expansion of pre-existing muscle resident macrophages through colony-stimulating factor 1 (CSF-1)/CSF-1 receptor (CSF-1R) signaling. (See attached “Wang et al_PNAS Manuscript” for details).

CSF-1R inhibition alone is not sufficient to improve the dystrophic phenotype of DMD. The therapeutic potential of CSF-1R inhibition has been explored in the DMD mouse model. Long-term inhibition of CSF-1R alone showed no notable effect on the myofiber size or maximum tetanus force production of mdx diaphragm muscle (12). It has been reported that CSFIR inhibition does not significantly impact on blood monocytes (75) or monocytes-derived infiltrating macrophages (12, 76). Therefore, CSF-1R inhibition appears mainly affects resident macrophages. Since monocytes-derived infiltrating macrophages contribute significantly to the diaphragm dystrophy in the DMD mouse models (8, 9) and resident macrophages expand to compensate when inflammatory monocyte/macrophage infiltration is blocked (Wang et at_PNAS Manuscript), simultaneous targeting of both monocytes-derived infiltrating macrophages (by CCR2 inhibition) and pre-existing resident macrophages (by CSF-1R inhibition) is likely required to achieve sufficient and long-lasting suppression of intramuscular macrophage accumulation to improve mdx^{5cv} diaphragm dystrophy.

In summary, the goal of our research is to suppress the accumulation of macrophages to reduce inflammation and improve dystrophic phenotype in DMD muscles through combinatorial inhibition of both CCR2 and CSF-1R signaling.

We focused our research on DMD mouse model mdx^{5cv} mice. To inhibit CCR2 signaling, we used both CCR2 deficient mdx^{5cv} mice (mdx^{5cv}/Ccr2^{-/-}) and CCR2-specific pharmacological inhibitor PF-04136309, which is a potent, selective, and orally bioavailable CCR2 antagonist for both human and mouse (77). PF-4136309 exhibits>100-fold selectivity over other homologous chemokine receptors and has an oral bioavailability of 47% in mice. It has been used for both mouse studies (78-80) and human clinical trials (81, 82), showing good tolerance and efficacy. To inhibit CSF-1R signaling, we used CSF-1R-specific pharmacological inhibitor PLX5622, which is derived from the most used agent for clinical studies PLX3397. PLX3397 is an orally administered small molecule tyrosine kinase inhibitor with selective activity against CSF-1R, KIT proto-oncogene receptor tyrosine kinase (KIT), and FMS-like tyrosine kinase 3 harboring an internal tandem duplication mutation (FLT 3-ITD). In 2019, PLX 3397 (Pexidartinib) was approved by US FDA for the treatment of adult patients with symptomatic tenosynovial giant cell tumor (55). PLX5622, a new derivative of PLX3397, shows a much higher selectivity than PLX3397 for CSF-1R over KIT and FLT3 (>20-fold) (56). Clinical data on the tolerability of CSFIR targeting agents show a favorable safety profile (54). Both PLX5622 and PLX3397 also show good tolerance in mouse studies with long treatment for 6 to 8 months (56, 57). PLX 5622 has been shown to effectively deplete MPs in vivo, especially the resident MP populations, in multiple disease models (56, 58-61), delaying the onset of experimental autoimmune encephalomyelitis (EAE) (59) and impairing parenchymal plaque development in an Alzheimer's disease model (56).

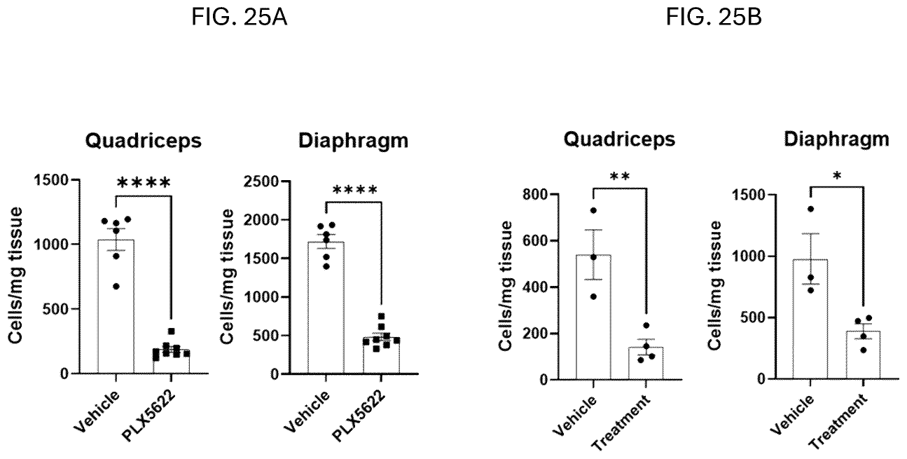

Mdx^{5cv}/Ccr2^{-/-} mice were given PLX5622 (150 mg freebase/kg body weight daily) orally starting at 4 weeks of age. Mdx^{5cv} mice were given both PLX 5622 (150 mg freebase/kg body weight daily) and PF-4136309 (100 mg/kg body weight daily) orally starting at 4 weeks of age. Quadriceps (limb muscle) and diaphragm (respiratory muscle showing persistent inflammation and progressive fibrosis) were collected at 14 weeks of age. No significant toxic side effect was noticed by checking mice body weight and multiple organs. The density of intramuscular macrophages was analyzed by both flow cytometry (FACS) and immunostaining. As shown by the figure below, macrophage density in both quadriceps and diaphragm from mdx^{5cv}/Ccr2^{-/-} mice (lacking CCR2 signaling) treated with CSF-1R inhibitor PLX5622 was significantly lower compared to that treated with vehicle control (FIG. 25A). Similarly, combinatorial inhibition of both CCR2 (by PF-4136309) and CSF-1R (by PLX5622) signaling also significantly reduced the density of intramuscular macrophages in mdx^{5cv} mice. Therefore, combinatorial inhibition of both CCR2 and CSF-1R can effectively suppress the accumulation of intramuscular macrophages in the dystrophic muscles of DMD mouse model, representing a viable treatment strategy for reducing muscle inflammation and improving muscular dystrophy phenotype.

FIG. 25. Quantified results of flow cytometry analysis of intramuscular macrophages from the quadriceps and diaphragm of (A) mdx^{5cv}/Ccr2^{-/-} mice receiving CCR2 inhibitor PF-4136309 and (B) mdx5cv mice receiving both CCR2 inhibitor PF-4136309 and CSF-1R inhibitor PLX5622.

### REFERENCES

1. A. E. Emery, The muscular dystrophies. BMJ 317, 991-995 (1998).
2. D. Duan, N. Goemans, S. Takeda, E. Mercuri, A. Aartsma-Rus, Duchenne muscular dystrophy. Nat Rev Dis Primers 7, 13 (2021).
3. H. H. Stedman et al., The mdx mouse diaphragm reproduces the degenerative changes of Duchenne muscular dystrophy. Nature 352, 536-539 (1991).
4. J. V. Hartel, J. A. Granchelli, M. S. Hudecki, C. M. Pollina, L. E. Gosselin, Impact of prednisone on TGF-betal and collagen in diaphragm muscle from mdx mice. Muscle Nerve 24, 428-432 (2001).
5. K. J. Nowak, K. E. Davies, Duchenne muscular dystrophy and dystrophin: pathogenesis and opportunities for treatment. EMBO Rep 5, 872-876 (2004).
6. P. Huang et al., Impaired respiratory function in mdx and mdx/utrn(+/−) mice. Muscle Nerve 43, 263-267 (2011).
7. P. Huang, X. S. Zhao, M. Fields, R. M. Ransohoff, L. Zhou, Imatinib attenuates skeletal muscle dystrophy in mdx mice. Faseb J 23, 2539-2548 (2009).
8. K. Mojumdar et al., Inflammatory monocytes promote progression of Duchenne muscular dystrophy and can be therapeutically targeted via CCR2. EMBO molecular medicine 6, 1476-1492 (2014).
9. W. Zhao, X. Wang, R. M. Ransohoff, L. Zhou, CCR2 deficiency does not provide sustained improvement of muscular dystrophy in mdx5cv mice. Faseb J 31, 35-46 (2017).
10. G. Juban, B. Chazaud, Metabolic regulation of macrophages during tissue repair: insights from skeletal muscle regeneration. FEBS Lett 591, 3007-3021 (2017).
11. H. Du et al., Macrophage-released ADAMTS 1 promotes muscle stem cell activation. Nat Commun 8, 669 (2017).
12. F. Babaeijandaghi et al., Metabolic reprogramming of skeletal muscle by resident macrophages points to CSFIR inhibitors as muscular dystrophy therapeutics. Sci Transl Med 14, eabg7504 (2022).
13. X. Wang, L. Zhou, The Many Roles of Macrophages in Skeletal Muscle Injury and Repair. Front Cell Dev Biol 10, 952249 (2022).
14. L. Zhou et al., Haploinsufficiency of utrophin gene worsens skeletal muscle inflammation and fibrosis in mdx mice. J Neurol Sci 264, 106-111 (2008).
15. L. Zhou et al., Temporal and spatial mRNA expression patterns of TGF-beta 1, 2, 3 and TbetaRI, II, III in skeletal muscles of mdx mice. Neuromuscul Disord 16, 32-38 (2006).
16. F. Geissmann, S. Jung, D. R. Littman, Blood monocytes consist of two principal subsets with distinct migratory properties. Immunity 19, 71-82 (2003).
17. V. Contreras-Shannon et al., Fat accumulation with altered inflammation and regeneration in skeletal muscle of CCR2-/-mice following ischemic injury. Am J Physiol Cell Physiol 292, C953-967 (2007).
18. H. Lu, D. Huang, R. M. Ransohoff, L. Zhou, Acute skeletal muscle injury: CCL2 expression by both monocytes and injured muscle is required for repair. Faseb J 25, 3344-3355 (2011).
19. H. Lu et al., Macrophages recruited via CCR2 produce insulin-like growth factor-1 to repair acute skeletal muscle injury. Faseb J 25, 358-369 (2011).
20. G. Bajpai et al., Tissue Resident CCR2− and CCR2+ Cardiac Macrophages Differentially Orchestrate Monocyte Recruitment and Fate Specification Following Myocardial Injury. Circ Res 124, 263-278 (2019).
21. L. Arnold et al., Inflammatory monocytes recruited after skeletal muscle injury switch into antiinflammatory macrophages to support myogenesis. J Exp Med 204, 1057-1069 (2007).
22. C. Shi, E. G. Pamer, Monocyte recruitment during infection and inflammation.

Nat Rev Immunol 11, 762-774 (2011).
23. T. Varga et al., Tissue LyC 6-macrophages are generated in the absence of circulating LyC6-monocytes and Nur77 in a model of muscle regeneration. J Immunol 191, 5695-5701 (2013).
24. X. Wang et al., Heterogeneous origins and functions of mouse skeletal muscle-resident macrophages. Proc Natl Acad Sci U S A 117, 20729-20740 (2020).
25. R. N. Hanna et al., The transcription factor NR4A1 (Nur 77) controls bone marrow differentiation and the survival of Ly6C-monocytes. Nat Immunol 12, 778-785 (2011).
26. G. Hoeffel, F. Ginhoux, Fetal monocytes and the origins of tissue-resident macrophages. Cell Immunol 330, 5-15 (2018).
27. X. Wang, L. Zhou, The multifaceted role of macrophages in homeostatic and injured skeletal muscle. Front Immunol 14, 1274816 (2023).
28. S. W. Boyer, A. V. Schroeder, S. Smith-Berdan, E. C. Forsberg, All hematopoietic cells develop from hematopoietic stem cells through Flk2/Flt3-positive progenitor cells. Cell Stem Cell 9, 64-73 (2011).
29. K. Mulder et al., Cross-tissue single-cell landscape of human monocytes and macrophages in health and disease. Immunity 54, 1883-1900 e1885 (2021).
30. S. A. Dick et al., Three tissue resident macrophage subsets coexist across organs with conserved origins and life cycles. Sci Immunol 7, eabf7777 (2022).
31. T. Hendrikx et al., Soluble TREM 2 levels reflect the recruitment and expansion of TREM2(+) macrophages that localize to fibrotic areas and limit NASH. J Hepatol 77, 1373-1385(2022).
32. K. Hoeft et al., Platelet-instructed SPP1(+) macrophages drive myofibroblast activation in fibrosis in a CXCL4-dependent manner. Cell Rep 42, 112131 (2023).
33. T. Fabre et al., Identification of a broadly fibrogenic macrophage subset induced by type 3 inflammation. Sci Immunol 8, eadd8945 (2023).
34. L. Boring et al., Impaired monocyte migration and reduced type 1 (Th1) cytokine responses in C-C chemokine receptor 2 knockout mice. J Clin Invest 100, 2552-2561 (1997).
35. D. R. Lemos et al., Nilotinib reduces muscle fibrosis in chronic muscle injury by promoting TNF-mediated apoptosis of fibro/adipogenic progenitors. Nature medicine 21, 786-794 (2015).
36. S. A. Villalta, H. X. Nguyen, B. Deng, T. Gotoh, J. G. Tidball, Shifts in macrophage phenotypes and macrophage competition for arginine metabolism affect the severity of muscle pathology in muscular dystrophy. Hum Mol Genet 18, 482-496 (2009).
37. S. A. Villalta et al., Interleukin-10 reduces the pathology of mdx muscular dystrophy by deactivating MI macrophages and modulating macrophage phenotype. Hum Mol Genet 20, 790-805 (2011).
38. J. Capote et al., Osteopontin ablation ameliorates muscular dystrophy by shifting macrophages to a pro-regenerative phenotype. J Cell Biol 213, 275-288 (2016).
39. G. Ji et al., Effect of AAV 9-hIGF-1 on inflammatory reaction in mdx mice and its mechanism. Am J Transl Res 12, 4488-4497 (2020).
40. J. G. Tidball, S. A. Villalta, Regulatory interactions between muscle and the immune system during muscle regeneration. Am J Physiol Regul Integr Comp Physiol 298, R1173-1187 (2010).
41. J. Munoz-Garcia et al., The twin cytokines interleukin-34 and CSF-1: masterful conductors of macrophage homeostasis. Theranostics 11, 1568-1593 (2021).
42. C. Moratal et al., IL- 1beta-and IL-4- polarized macrophages have opposite effects on adipogenesis of intramuscular fibro-adipogenic progenitors in humans. Sci Rep 8, 17005 (2018).
43. F. Babaeijandaghi et al., DPPIV(+) fibro-adipogenic progenitors form the niche of adult skeletal muscle self-renewing resident macrophages. Nat Commun 14, 8273 (2023).
44. X. Wang et al., Diverse effector and regulatory functions of fibro/adipogenic progenitors during skeletal muscle fibrosis in muscular dystrophy. iScience 26, 105775 (2023).
45. M. B. Buechler et al., Cross-tissue organization of the fibroblast lineage. Nature 593, 575-579 (2021).
46. N. R. Johnson et al., CSFIR inhibitors induce a sex-specific resilient microglial phenotype and functional rescue in a tauopathy mouse model. Nat Commun 14, 118 (2023).
47. E. Spangenberg et al., Sustained microglial depletion with CSF 1R inhibitor impairs parenchymal plaque development in an Alzheimer's disease model. Nat Commun 10, 3758 (2019).
48. A. W. Joe et al., Muscle injury activates resident fibro/adipogenic progenitors that facilitate 679 myogenesis. Nat Cell Biol 12, 153-163 (2010).
49. O. Contreras, F. M. V. Rossi, M. Theret, Origins, potency, and heterogeneity of skeletal muscle 681 fibro-adipogenic progenitors-time for new definitions. Skelet Muscle 11, 16(2021).
50. A. Uezumi et al., Fibrosis and adipogenesis originate from a common mesenchymal progenitor in 683 skeletal muscle. J Cell Sci 124, 3654-3664 (2011).
51. O. Contreras et al., Cross-talk between TGF-beta and PDGFRalpha signaling pathways regulates 685 the fate of stromal fibro-adipogenic progenitors. J Cell Sci 132 (2019).
52. D. L. Rebolledo, K. E. Lipson, E. Brandan, Driving fibrosis in neuromuscular diseases: Role and 687 regulation of Connective tissue growth factor (CCN2/CTGF). Matrix Biol Plus 11, 100059 (2021).
53. J. Tonkin et al., Monocyte/Macrophage-derived IGF-1 Orchestrates Murine Skeletal Muscle Regeneration and Modulates Autocrine Polarization. Mol Ther 23, 1189-1200(2015).
54. X. S. Revelo et al., Cardiac Resident Macrophages Prevent Fibrosis and Stimulate Angiogenesis. Circ Res 129, 1086-1101 (2021).
55. M. Theret, F. M. V. Rossi, O. Contreras, Evolving Roles of Muscle-Resident Fibro-Adipogenic Progenitors in Health, Regeneration, Neuromuscular Disorders, and Aging. Front Physiol 12, 673404 (2021).
56. A. Uezumi, M. Ikemoto-Uezumi, K. Tsuchida, Roles of nonmyogenic mesenchymal progenitors in pathogenesis and regeneration of skeletal muscle. Front Physiol 5, 68 (2014).
57. O. Contreras, D. L. Rebolledo, J. E. Oyarzun, H. C. Olguin, E. Brandan, Connective tissue cells expressing fibro/adipogenic progenitor markers increase under chronic damage: relevance in fibroblast-myofibroblast differentiation and skeletal muscle fibrosis. Cell and tissue research 10.1007/s00441-015-2343-0 (2016).
58. M. Theret, M. Saclier, G. Messina, F. M. V. Rossi, Macrophages in Skeletal Muscle Dystrophies, An Entangled Partner. J Neuromuscul Dis 9, 1-23 (2022).
59. G. Coulis et al., Single-cell and spatial transcriptomics identify a macrophage population associated with skeletal muscle fibrosis. Sci Adv 9, eadd9984 (2023).
60. L. G. M. Heezen et al., Spatial transcriptomics reveal markers of histopathological changes in Duchenne muscular dystrophy mouse models. Nat Commun 14, 4909 (2023).
61. A. E. H. Emery, Duchenne Muscular Dystrophy (Oxford University Press, Oxford, 1993).
62. E. P. Hoffman, R. H. Brown, Jr., L. M. Kunkel, Dystrophin: the protein product of the Duchenne muscular dystrophy locus. Cell 51, 919-928 (1987).
63. I. Desguerre et al., Endomysial fibrosis in Duchenne muscular dystrophy: a marker of poor outcome associated with macrophage alternative activation. Journal of neuropathology and experimental neurology 68, 762-773 (2009).
64. K. D. Huebner, D. S. Jassal, O. Halevy, M. Pines, J. E. Anderson, Functional resolution of fibrosis in mdx mouse dystrophic heart and skeletal muscle by halofuginone. American journal of physiology. Heart and circulatory physiology 294, H1550-1561 (2008).
65. C. F. Spurney et al., Losartan decreases cardiac muscle fibrosis and improves cardiac function in dystrophin-deficient mdx mice. Journal of cardiovascular pharmacology and therapeutics 16, 87-95 (2011).
66. A. P. Taniguti, A. Pertille, C. Y. Matsumura, H. Santo Neto, M. J. Marques, Prevention of muscle fibrosis and myonecrosis in mdx mice by suramin, a TGF-betal blocker. Muscle Nerve 43, 82-87 (2011).
67. T. Turgeman et al., Prevention of muscle fibrosis and improvement in muscle performance in the mdx mouse by halofuginone. Neuromuscul Disord 18, 857-868 (2008).
68. L. Cordier et al., Rescue of skeletal muscles of gamma-sarcoglycan-deficient mice with adeno-associated virus-mediated gene transfer. Molecular therapy: the journal of the American Society of Gene Therapy 1, 119-129 (2000).
69. C. Gargioli, M. Coletta, F. De Grandis, S. M. Cannata, G. Cossu, PIGF-MMP-9-expressing cells restore microcirculation and efficacy of cell therapy in aged dystrophic muscle. Nature medicine 14, 973-978 (2008).
70. T. A. Wynn, K. M. Vannella, Macrophages in Tissue Repair, Regeneration, and Fibrosis. Immunity 44, 450-462 (2016).
71. L. E. Olson, P. Soriano, Increased PDGFRalpha activation disrupts connective tissue development and drives systemic fibrosis. Dev Cell 16, 303-313 (2009).
72. G. Goldspink, K. Fernandes, P. E. Williams, D. J. Wells, Age-related changes in collagen gene expression in the muscles of mdx dystrophic and normal mice. Neuromuscul Disord 4, 183-191 (1994).
73. N. Beastrom et al., mdx((5)cv) mice manifest more severe muscle dysfunction and diaphragm force deficits than do mdx Mice. The American journal of pathology 179, 2464-2474 (2011).
74. C. Auffray et al., Monitoring of blood vessels and tissues by a population of monocytes with patrolling behavior. Science 317, 666-670 (2007).
75. V. Bellver-Landete et al., Microglia are an essential component of the neuroprotective scar that forms after spinal cord injury. Nat Commun 10, 518 (2019).
76. A. Montilla et al., Microglia and meningeal macrophages depletion delays the onset of experimental autoimmune encephalomyelitis. Cell Death Dis 14, 16 (2023).
77. C. B. Xue et al., Discovery of INCB 8761/PF-4136309, a Potent, Selective, and Orally Bioavailable CCR2 Antagonist. ACS Med Chem Lett 2, 913-918 (2011).
78. J. B. Mitchem et al., Targeting tumor-infiltrating macrophages decreases tumor-initiating cells, relieves immunosuppression, and improves chemotherapeutic responses. Cancer Res 73, 1128-1141 (2013).
79. D. E. Sanford et al., Inflammatory monocyte mobilization decreases patient survival in pancreatic cancer: a role for targeting the CCL2/CCR2 axis. Clin Cancer Res 19, 3404-3415 (2013).
80. X. Wu et al., A Small Molecule CCR2 Antagonist Depletes Tumor Macrophages and Synergizes with Anti-PD-1 in a Murine Model of Cutaneous T-Cell Lymphoma (CTCL). J Invest Dermatol 140, 1390-1400 e1394 (2020).
81. T. M. Nywening et al., Targeting tumour-associated macrophages with CCR2 inhibition in combination with FOLFIRINOX in patients with borderline resectable and locally advanced pancreatic cancer: a single-centre, open-label, dose-finding, non-randomised, phase 1b trial. Lancet Oncol 17, 651-662 (2016).
82. M. Noel et al., Phase 1 b study of a small molecule antagonist of human chemokine (C-C motif) receptor 2 (PF-04136309) in combination with nab-paclitaxel/gemcitabine in first-line treatment of metastatic pancreatic ductal adenocarcinoma. Invest New Drugs 38, 800-811 (2020).
83. M. Lenardo et al., Mature T lymphocyte apoptosis-- immune regulation in a dynamic and 362 unpredictable antigenic environment. Annu Rev Immunol 17, 221-253 (1999).
84. O. Contreras, F. M. Rossi, E. Brandan, Adherent muscle connective tissue fibroblasts are 364 phenotypically and biochemically equivalent to stromal fibro/adipogenic progenitors. Matrix 365 Biol Plus 2, 100006 (2019).

All documents cited in this specification are incorporated herein by reference are incorporated by reference. While the invention has been described with reference to particular embodiments, it will be appreciated that modifications can be made without departing from the spirit of the invention. Such modifications are intended to fall within the scope of the appended claims.

1. A method for treating chronic muscle inflammation, the method comprising systemically administering to subject in a need thereof a therapeutically effective amount of at least one compound that disrupts monocyte recruitment and/or macrophage expansion in response to muscle injury.

2. The method of claim 1, wherein the systemic administration comprises oral administration, subcutaneous injection, and/or intravenous injection.

3. The method of claim 1 wherein the method comprises disrupting monocyte recruitment and macrophage expansion in response to muscle injury.

4. The method of claim 1, wherein the chronic muscle inflammation is associated with muscular dystrophy, optionally Duchenne Muscular Dystrophy (DMD).

5. The method of claim 1, wherein the at least one compound is a small molecule inhibitor, a peptide inhibitor, a protein inhibitor, an antibody, optionally a neutralizing antibody, a nucleic acid molecule inhibitor, optionally a small interfering RNA (siRNA).

6. The method of claim 1, wherein the method comprises disrupting monocyte recruitment, optionally wherein the method comprises blocking recruitment of blood Ly6C^{hi} inflammatory monocyte by injured muscle.

7. The method of claim 1, wherein the method comprises decreasing Ly6C^{hi} macrophage infiltration.

8. The method of claim 1, wherein the at least one compound inhibits C-C motif chemokine receptor 2 (CCR2) signaling.

9. The method of claim 1, wherein the at least one compound is a CCR2 inhibitor.

10. The method of claim 1, wherein the at least one compound is a C-C motif chemokine ligand 2 (CCL2; MCP-1), C-C motif chemokine ligand 8 (CCL8; MCP-2), C-C motif chemokine ligand 7 (CCL7; MCP-3), and/or C-C motif chemokine ligand 13 (CCL13; MCP-4) inhibitor.

11. The method of claim 1, wherein the at least one compound disrupts CCR2-CCL2 binding.

12. The method of claim 1, wherein the method comprises disrupting macrophage expansion.

13. The method of claim 1, wherein the method comprises decreasing Ly6C^{lo} macrophage expansion.

14. The method of claim 1, wherein the at least one compound inhibits a colony stimulating factor-1 receptor (CSF-1R) signaling.

15. The method of claim 1, wherein the at least one compound is a CSF-1R inhibitor.

16. The method of claim 1, wherein the at least one compound is a colony stimulating factor-1 (CSF-1) inhibitor.

17. The method of claim 1, wherein the at least one compound disrupts CSF-1R-CSF1 binding.

18. The method of claim 1, wherein the at least one compound is a CCR2 inhibitor that is PF-04136309, TAK-652, RS504393, MK0812, PF-04634817, or INCB3344.

19. The method of claim 1, wherein the at least one compound is a CSF-1R inhibitor that is PLX3397, PLX5622, BLZ945, or PLX73086.

20. The method of claim 1, wherein the method comprises administering to subject in a need thereof a therapeutically effective amount of at least one compound that inhibits CCR2 signaling and at least one compound that inhibits CSF-1R signaling.

21. A regimen for treating chronic muscle inflammation, the regimen comprising administering to subject in a need thereof a therapeutically effective amount of at least one compound that disrupts monocyte recruitment in response to muscle injury and at least one compound that disrupts macrophage expansion in response to muscle injury.

22. The regimen of claim 21, wherein the regimen comprises administering of the at least one compound that disrupts monocyte recruitment and at least one compound that disrupts macrophage expansion simultaneously.

23. The regimen of claim 21, wherein the regimen comprises first administering of the at least one compound that disrupts monocyte recruitment, followed by administering at least one compound that disrupts macrophage expansion.

24. The regimen of claim 21, wherein the at least one compound inhibits C-C motif chemokine receptor 2 (CCR2) signaling.

25. The regimen of claim 21, wherein the at least one compound inhibits a colony stimulating factor-1 receptor (CSF-1R) signaling.
